(19) United States
(12) Patent Application Publication
(10) Pub. No.: US 2011/0008302 A1
(43) Pub. Date: Jan. 13, 2011

(54) Compositions and methods for immortalizing cells and screening for anti-cancer agents

(76) Inventors: Philip N. Tsichlis, Winchester, MA (US); Susan E. Bear, Winchester, MA (US); Raymond Pfau, Boston, MA (US); Filippos Kottakis, Malden, MA (US)

Correspondence Address: LAWSON & WEITZEN, LLP, 88 BLACK FALCON AVE, SUITE 345, BOSTON, MA, 02210, US

(21) Appl. No.: 12/795,374

(22) Filed: Jun. 7, 2010

Related U.S. Application Data
(63) Continuation-in-part of application No. PCT/US2008/013475, filed on Dec. 8, 2008.
(60) Provisional application No. 61/005,799, filed on Dec. 7, 2007.

(51) Int. Cl. A61K 35/12; C12Q 1/00; C12Q 1/68; G01N 33/53; C12Q 1/32; C12N 15/63; C12N 5/00; C40B 30/04; C40B 30/06; A61K 31/713; C07K 2/00; C07K 16/18; A61P 35/00; A61P 35/04; A61P 25/18; A61P 7/06; A61P 25/28

(52) U.S. Cl. 424/93.21; 435/4; 435/6; 435/7.1; 435/26; 435/440; 435/375; 435/377; 435/320.1; 506/9; 506/10; 514/44 A; 530/300; 530/387.9

(57) ABSTRACT

An embodiment of the invention provides methods and compositions for immortalizing cells, and in another embodiment for reversing immortalization of cells. Methods for screening for anti-cancer agents by identifying compounds that bind to and inhibit activity of an Ndy protein are also provided. Novel cancer diagnostic and prognostic methods using Ndy1, Ezh2 and miR-101 are shown with samples of tissue or cell lines from cancers of lung, colon, ovary, bladder and transitional cell carcinoma.

### RELATED APPLICATIONS

This application is a continuation-in-part of and claims the benefit of PCT application PCT/US2008/013475 filed Dec. 8, 2008, and claims the benefit of U.S. provisional patent application Ser. No. 61/005,799 filed Dec. 7, 2007, entitled, “Compositions and methods for immortalizing cells and for screening for anti-cancer agents”, each of which is hereby incorporated herein by reference in its entirety.

### GOVERNMENT SUPPORT

The invention herein was made in part with support of grants from the National Institutes of Health R01CA109747 and P30DK34928. The government has certain rights in the invention.

### TECHNICAL FIELD

Compositions and methods for immortalizing or for inhibiting growth of cells by targeting the Rb and p53 pathways with the protein encoded by the Ndy1 gene are provided, and the protein presents a new target for identifying anti-cancer compounds.

### BACKGROUND

Cancer remains a major public health problem among adults, and is the leading disease-related cause of death in children. New functions associated with oncogenes and tumor suppressors are discovered by a variety of different phenotypes, and isolation and characterization of these genes offer opportunity for further insight into cell processes, pathologies, and targets for drug discovery.

Core histones contain a “histone fold” globular domain, which is responsible for histone-DNA and histone-histone interactions, and N-terminal and C-terminal tails. Histone tails contain sites that are targets of various posttranslational modifications, including phosphorylation, acetylation, methylation and ubiquitination (Berger, S. L. (2002) Curr Opin Genet Dev 12, 142-8). Postranslational modifications of histone tails regulate the interaction of nucleosomes with other nucleosomes and with linker DNA, and direct the folding of chromatin into a higher order structure (Hansen, J. C. (2002) Annu Rev Biophys Biomol Struct 31, 361-92). The same modifications regulate the binding of various nonhistone chromatin-associated proteins (Strahl, B. D. et al. (2000) Nature 403, 41-5). As a result, enzymes involved in the posttranslational modification of histone tails, in combination with chromatin remodeling enzymes (Kwon, C. S. et al. (2007) Trends Genet 23, 403-12), regulate transcription and other chromatin-dependent activities. Modification of histone tails is a dynamic process with all modifications being transient (Berger, S. L. (2002) Curr Opin Genet Dev 12, 142-8). The most recently discovered enzyme group responsible for the reversal of a histone modification is that of histone demethylases (Shi, Y., et al. (2004) Cell 119, 941-53; Tsukada, Y. et al. (2006) Nature 439, 811-6; Klose, R. J. et al. (2006) Nat Rev Genet 7, 715-27).

Histone methylation on lysine and arginine residues contributes to the regulation of gene expression, dosage compensation and epigenetic memory (Berger, S. L. (2002) Curr Opin Genet Dev 12, 142-8, 11; Martin, C. et al. (2005) Nat Rev Mol Cell Biol 6, 838-49). The functional consequences of histone methylation depend on the methylation site. Thus, whereas methylation at H3K9, H3K27 and H4K20 is usually associated with transcriptional repression, methylation at H3K4, H3K36 and H3K79 is associated with transcriptional activation (Martin, C. et al. (2005) Nat Rev Mol Cell Biol 6, 838-49). The stoichiometry of histone methylation at a given site is also functionally important, because the methylation-directed binding of transcriptional regulators to methylated histones is stoichiometry-dependent (Wysocka, J. et al. (2006) Nature 442, 86-90).

There are three distinct classes of histone demethylases (Klose, R. J. et al. (2006) Nat Rev Genet 7, 715-27). The largest of these classes consists of enzymes containing a JmjC domain, a homolog of the cupin metalloenzymes (Klose, R. J. et al. (2006) Nat Rev Genet 7, 715-27; Clissold, P. M. et al. (2001) Trends Biochem Sci 26, 7-9). The JmjC domain-containing enzymes catalyze demethylation through an oxidative reaction which depends on two co-factors, iron Fe(II) and α-ketoglutarate. Specifically, these enzymes catalyze the hydroxylation of mono, di or trimethylated lysine in histone tails, giving rise to an unstable hydroxyl-methyl group, which is released spontaneously as formaldehyde (Tsukada, Y. et al. (2006) Nature 439, 811-6, Klose, R. J. et al. (2006) Nat Rev Genet 7, 715-27).

Some JmjC domain-containing histone demethylases have been linked to cancer (Yamane, K. et al. (2007) Mol Cell 25, 801-12; Cloos, P. A. et al. (2006) Nature 442, 307-11; Suzuki, T. et al. (2006) Embo J 25, 3422-31) However, the relationship has not been established, either specifically or mechanistically, and in view of different forms of these proteins. Cancer remains a leading cause of death in adults and children, and the mechanisms of oncogenic activities of JmjC domain-containing demethylases are not well understood. There is a need for new targets to screen for lead compounds to obtain compositions and methods for treating cancer.

### SUMMARY OF EMBODIMENTS

An embodiment of the invention herein provides a pharmaceutical composition for inhibiting immortalization and stimulating differentiation of a cell, the composition having an effective dose of at least one mammalian short form Ndy protein-related composition selected from the group of: a short form Ndy1 protein; a short form Ndy2 protein; a vector encoding a short form Ndy1 nucleotide sequence; a vector encoding a short form Ndy2 nucleotide sequence; a modulator of short form Ndy1 expression; a modulator of short form Ndy2 expression; wherein the short form Ndy protein, vector and modulator function to increase cellular amount or activity of short form Ndy protein lacking a functional JmjC domain thereby lacking demethylase activity, and the short form Ndy protein further inhibits histone demethylase Ndy long form expression or activity, and so by this method the immortalization of the cell is inhibited and differentiation is stimulated.

A related embodiment provides the pharmaceutical composition further including a pharmaceutical salt, buffer, or emollient. Each of a long form and a short form Ndy protein-related compositions are accordingly provided in an effective ratio to regulate amount of histone demethylation and thereby regulate gene expression, so that a higher ratio of long form to short form promotes immortalization, and a lower ratio promotes differentiation and senescence. For example, immortalization is promoted by a ratio of greater than about 1:1, about 2:1, about 5:1, or about 10:1 of long form to short form, and differentiation and senescence are promoted by a ratio of less than about 1:1, about 1:2, about 1:5, or about 1:10 of long form to short form.

Another embodiment of the invention herein provides a method of immortalizing a cell including steps of:

contacting the cell with a vector carrying a nucleotide sequence selected from the group encoding: an Ndy1 gene operably linked to regulatory signals promoting expression of Ndy1 gene, wherein the Ndy1 gene includes a functional JmjC domain; an Ndy2 gene operably linked to regulatory signals to promote expression of the Ndy2 gene, wherein the Ndy2 gene includes a functional JmjC domain; and a negative modulator of expression of a short form Ndy1 and/or Ndy2 gene; and,

culturing and measuring immortalization of the cell during passaging and storage.

In a related embodiment the method further includes, after culturing, implanting the cell in vivo. In a related embodiment the method further includes, prior to contacting, obtaining the cell in vivo from a subject suffering from a senescence condition. For example, the senescence condition is neurological, muscular, hematopoietic, or dermatological. For example, the condition is selected from Alzheimers, pre-Alzheimers, amnesia, psychosis, muscular dystrophy, myotonic dystrophy, sickle cell anemia, thallasemia, and progeria.

Another embodiment of the invention provides a method of promoting at least one of cell differentiation and cell senescence, the method including steps of:

contacting the cell with a vector carrying a nucleotide sequence selected from: a short form of an Ndy1 gene operably linked to regulatory signals to promote expression of the Ndy1 gene, wherein the Ndy1 gene lacks a functional JmjC domain; a short form of an Ndy2 gene operably linked to regulatory signals to promote expression of the Ndy2 gene, wherein the Ndy2 gene lacks information encoding a functional JmjC domain; and a modulator that upregulates expression of at least one of the short form Ndy1 and the short form Ndy2 gene; and,

culturing the cell and analyzing an amount of at least one of differentiation and senescence in cultured amplified cells. In a related embodiment the method further includes, prior to contacting, obtaining the cell from a subject suffering from a cancer or a neoplastic condition. A related method further includes, after culturing, implanting the cells in vivo. For example, the cancer is a hematopoietic condition selected from a leukemia and a lymphoma. Alternatively, the cancer is selected from the group of prostate, testicular cancer, breast, colon, ovarian, bladder, transitional cell carcinoma, pancreatic, esophageal, lung, brain, melanoma, and basal cell carcinoma. In related embodiments, the method further includes treating the subject with an anti-tumor agent or procedure. For example, the anti-tumor agent or procedure is at least one of radiation, thermal disruption, and angiogenesis inhibition.

An embodiment of the invention herein provides a method of obtaining an anti-Ndy antibody having steps of:

contacting an animal with a peptide 907-KMRRKRRLVNKELSKC-921 (SEQ ID NO:2) or a fragment or analog thereof for a time sufficient for a serum sample from the animal to indicate increasing titers of the antibody and optimal production wherein the antibody binds with specificity and affinity to an Ndy protein; and,

recovering the antibody in serum from the animal at a time of optimal antibody production, thereby obtaining the anti-Ndy antibody. For example, the fragment is at least four amino acids to seven amino acids in length. Further, the antibody recognizes and binds to an Ndy protein from a mammal.

An embodiment of the invention provides a method of prognosing or diagnosing a cell or tissue for susceptibility to a cancer, the method having steps of:
contacting a test sample comprising the cell, tissue or an extract thereof with an anti-Ndy antibody or a nucleotide sequence encoding a portion of an Ndy gene under conditions for antigen-antibody binding or conditions of nucleotide hybridization, respectively; observing amount of binding of the antibody or hybridization of the nucleic acid to the test sample; and,

analyzing the amount in comparison to a control sample from a normal cell or tissue known to be negative for the cancer, wherein an greater amount of binding of the Ndy antigen or nucleic acid to the test sample in comparison to the control samples provides a prognosis or a diagnosis of susceptibility to cancer. The method in a related embodiment includes determining in the amount of a ratio of Ndy long form compared to Ndy short form, such that a greater amount of the long form compared to the short form is a prognosis or diagnosis of susceptibility to cancer.

Another embodiment of the invention herein provides a method of identifying from a library of compounds a compound capable of binding to and inhibiting activity of an Ndy protein, the method comprising:

contacting the compound to a first cell having an Ndy retroviral construct encoding a long Ndy form having a JmjC domain; and,

identifying the compound by observing a differentiation morphology of the first cell in comparison with that of a second cell which is a control having the identical Ndy retroviral construct and not so contacted with the compound, and further in comparison with a third cell which is a control identically contacted with the compound and lacking the retroviral construct, thereby identifying the compound as capable of binding to and inhibiting activity of the Ndy protein.

For example, each of the first cell, second cell and third cell is a plurality of cells, each in culture in a well of a multi-well culture dish. Further, observing in various embodiments includes measuring a marker of differentiation by at least one parameter which is immunologic, colorimetric, fluorimetric, fluorescent, radioactive, or enzymatic.

An embodiment of the invention herein provides a method of treating a subject having a cancer selected from a breast cancer, a testicular cancer, a leukemia and a lymphoma, the method including: contacting the subject with a vector carrying an siRNA that inhibits expression of an Ndy protein, in which the Ndy protein includes a JmjC domain; and, measuring inhibition by the siRNA of expression of endogenous Ndy protein and function or activity of the JmjC domain, and so by this method treating the subject.

An embodiment of the invention herein provides a method of identifying a compound capable of binding to and inhibiting activity of an Ndy protein, the method comprising:

contacting the compound to a first sample of an Ndy protein in an in vitro assay of an enzymatic reaction that includes at least one methylated substrate, and under conditions suitable for histone demethylation, in which the Ndy protein comprises a JmjC domain; and,

observing inhibition by the compound of an amount of enzymatic reaction product demethylated substrate, wherein the compound is identified as inhibiting the amount produced in the first sample, compared to that of a second control sample having identical Ndy protein and not so contacted with the compound, so that the compound decreases amount of demethylated product in the first sample compared to the second sample, thereby identifying the compound as inhibiting activity of the Ndy protein.

A related method includes observing a third sample which is a control that includes the substrate and is lacking the Ndy protein, so that the third sample is a control for determining spontaneous non-enzymatic background demethylation. For example, Ndy is a long form of Ndy1 or Ndy2. A related embodiment of the method further includes, prior to contacting, preparing the sample of Ndy protein from at least one of the group of: a crude cell extract; an enriched fraction cell extract by preparative immunoprecipitation; and a bacterially produced recombinant protein. In a related embodiment, the compound is present in a plurality of compositions in a sibling pool, and contacting the compound to the protein further involves a plurality of samples in a high throughput multi-well format. For example, the methylated substrate is bulk histone and observing comprises performing a western blot of an electrophoretogram. For an alternative or additional example, the methylated substrate is a di-methylated or a tri-methylated isolated synthetic peptide and observing is measuring a change in fluorescence of product formaldehyde by a glutathione-independent formaldehyde dehydrogenase which reduces NAD⁺ to NADH. For example, the di-methylated or tri-methylated isolated synthetic peptide is at least one of ART-K(me₃)-QTARKST and ATGGV-K(me₂)-KPHRY. For example, conditions suitable for histone demethylation comprise presence of α-ketoglutarate and an iron salt.

A related embodiment of the method further includes observing anti-cancer activity of the compound.

Another embodiment of the invention herein provides a method of treating a cancer or senescence condition of a cell, the method including the steps of formulating a composition having an Ndy amino acid sequence or a nucleotide sequence encoding an Ndy amino acid in a pharmaceutically acceptable buffer or salt, and contacting the cell with the composition. For example, the method further includes formulating the composition in an effective dose.

A method is provided herein for inhibiting growth of cells, the method including contacting the cells with an siRNA capable of inhibiting Ndy1 expression. For example, the siRNA includes nucleotide sequence 1433-GUGGACUCACCUUACCGAAUU-1454 (SEQ ID NO:1) or a portion thereof.

### BRIEF DESCRIPTION OF DRAWINGS

FIG. 1 is a set of drawings and photographs showing that provirus insertion activates the Ndy1 gene.

FIG. 1 panel A shows sites and orientation of provirus integration at the 5′ end of the Ndy1 gene on the top line. The numbers above the arrows showing the sites of provirus integration identify the tumors in which the integrations were detected. The lower line shows the domain structure of the Ndy1 protein.

FIG. 1 panel B shows the site and orientation of provirus integration at the 5′ end of the Ndy2 gene in tumor #4 on the upper line. The lower line shows the domain structure of the Ndy2 protein.

FIG. 1 panel C is a set of photo micrographs of NIH 3T3 cells that were infected with a MigR1 construct of Ndy1.HA. (Left) Two GFP-positive, infected cells. (Center) The same cells stained with an anti-HA antibody. (Right) Ndy1.HA-expressing cell stained with an anti-HA antibody and visualized by confocal microscopy. The darker spots correspond to nucleoli.

FIG. 1 panel D is a set of photographs showing, in the upper, a Northern blot of total cell RNA derived from normal rat thymus and the indicated tumors (R6 to 5677) probed with a full-length rat Ndy1 cDNA probe. The lower photographs show ethidium bromide staining of the gel (loading control).

FIG. 1 panel E is a set of photographs of Western blots of nuclear cell lysates from normal thymus and the indicated tumors probed with anti-Ndy1 and anti-CREB antibodies as indicated.

FIG. 2 is a drawing showing site and orientation of provirus integration at the 5′ end of the Phf2 (top) and Phf8 (bottom) genes in the MoMuLV-induced rat T cell lymphomas A7 and 16 respectively. Schematic diagrams of the intron-exon structures of each gene and domain composition of the encoded proteins is shown. The sites and transcriptional orientations of the integrated proviruses are shown by arrows.

FIG. 3 is a set of photographs of Northern and Western blots showing that Ndy1 is expressed in the testis, thymus and spleen and is overexpressed in MoMuLV-induced rat T cell lymphomas carrying a provirus at the 5′ end of the Ndy1 gene.

FIG. 3 panel A shows Northern blot of total cell RNA derived from the indicated rat tissues was probed with a full length rat Ndy1 cDNA probe (upper). The 28S ribosomal RNA, visualized by ethidium bromide (EtBr) staining, is presented as the loading control. FIG. 3 panel A shows Western blot of nuclear lysates derived from the indicated normal rat tissues was probed with anti-Ndy1 and anti-GAPDH antibodies as indicated (lower). GAPDH was used as the positive control.

FIG. 3 panel B shows Western blots of nuclear and cytoplasmic fractions of HEK293 cells transiently transfected with Ndy1 expression constructs were probed with the indicated antibodies (left). Western blot of soluble whole cell lysates and of the insoluble nuclear fraction of HEK293 cells transiently transfected with Ndy1 constructs were probed with the indicated antibodies (right).

FIG. 4 is a drawing and set of photographs showing aberrant MoMuLV-Ndy1 hybrid transcripts in tumors carrying an integrated provirus 5′ and in the same transcriptional orientation as the Ndy1 gene, encode a cytoplasmic and not a nuclear protein.

FIG. 4 panel A shows structure of MoMuLV-Ndy1 hybrid mRNA transcripts.

FIG. 4 panel B shows Western blots of cytoplasmic and nuclear cell lysates from MEFs infected with the indicated retrovirus constructs, were probed with anti-Ndy1, anti-tubulin (cytoplasmic protein) and anti-CREB (nuclear protein) antibodies.

FIG. 4 panel C shows MEFs infected with an Env-Ndy1.HA retroviral construct were stained with a monoclonal anti-HA antibody and they were visualized by fluorescent microscopy.

FIG. 5 is a set of drawing showing a summary of Ndy1 isoforms in Homo sapiens and Mus musculus and Rattus norvegicus

FIG. 6 is a set of photographs of Western blots showing expression of Ndy1.HA in MEFs infected with MigR1-Ndy1.HA or MigR1-Ndy2.HA constructs. Western blots of total cell lysates from the indicated MEF cultures were probed with an anti-HA or with an anti-Tubulin antibody.

FIG. 7 is a set of photomicrographs and line graphs showing that MEFs expressing Ndy1 or Ndy2 bypass replicative senescence and undergo immortalization.

FIG. 7 panel A shows β-galactosidase staining of 11th-passage MEFs infected with the MigR1 retrovirus vector (Left) or with a MigR1-Ndy1 construct (Right).

FIG. 7 panels B and C show MEFs infected with the MigR1 vector or MigR1-Ndy1 and MigR1-Ndy2 constructs. The graph shows the cumulative number of cells (ordinate) in each culture at sequential passages (abscissa).

FIG. 8 is a line graph showing that the Env-Ndy1 hybrid protein, which is localized in the cytoplasm rather than the nucleus, does not immortalize MEFs in culture. MEFs infected with the indicated retroviral constructs were passaged and the number of cells was monitored in culture as described herein.

FIG. 9 is a set of photographs and line graphs that show immortalization depends on the histone demethylase activities of Ndy1 and Ndy2.

FIG. 9 panel A is a photograph of Western blots of total cell lysates derived from MEFs infected with MigR1 or with wild-type or mutant MigR1-Ndy1.Myc. The blots were probed with anti-Ndy1 or with anti-GAPDH (loading control) antibodies as indicated. Ndy1-ΔPRR carries a deletion of the prolene-rich region, upstream of the F-box see FIG. 1 panel A. The LRR deletion mutant was also tested in separate analyses, and it was shown to immortalize MEFs as efficiently as the wild-type protein.

FIG. 9 panel B is a line graph of the MEFs shown in panel A passaged in culture. Graphs show the cumulative number of cells at each passage. The Ndy1.H283Y mutant was also tested in separate analyses, and it was shown to have the same dominant-negative phenotype as the Ndy1.Y221A mutant.

FIG. 9 panel C is a set of photographs of Western blots of cell lysates from cells infected with MigR1-Ndy1 or MigR1-ΔJmjC Ndy1 that were probed with the anti-Ndy1 antibody.

FIG. 9 panel D is a set of photomicrographs of MEFs infected with the indicated constructs that were passaged in culture.

FIG. 9 panel E is a line graph of MEFs infected with the indicated constructs, that were passaged in culture, showing cumulative numbers of cells at each passage.

FIG. 10 is a set of photographs and line graphs showing that endogenous Ndy1 physiologically inhibits replicative senescence in MEFs.

FIG. 10 panel A show the Ndy1 and scrambled siRNAs transfected into wild type or MigR1Ndy1.HA-infected MEFs. Western blots of transfected cell lysates were probed with the anti-Ndy1 antibody.

FIG. 10 panel A1 is photograph of a Western blot of cell lysates harvested 48 hours after the transfection.

FIG. 10 panel A2 shows a time course of exogenous Ndy1 starting with cell lysates harvested 24 hours after transfection.

FIG. 10 panel B is a set of line graphs of early passage wild type and MigR1-Ndy1.HA-infected MEFs transfected with Ndy1 or control siRNAs and passaged twice every 72 hours. siRNA transfection was repeated after the first passage at the 72 hour time point. Cells were counted and the numbers were plotted as indicated.

FIG. 10 panel C is a set of photomicrographs of early passage wild type and MigR1-Ndy1.HA-infected MEFs were transfected with Ndy1 or control siRNA and they were visualized by light microscopy.

FIG. 10 panel D is a set of photomicrographs of the same cells as in panel C that were stained for β-galactosidase.

FIG. 11 is a line graph showing that overexpression of Ndy1 does not immortalize human IMR90 fibroblasts in culture. IMR90 cells infected with the indicated retroviral constructs were passaged and the number of cells was monitored in culture as described herein.

FIG. 12 is a set of photographs and a line graph showing that Ndy1 promotes immortalization by targeting the Rb and p53 pathways.

FIG. 12 panel A is a photograph of Western blots of MEFs infected with the indicated constructs that were probed with the indicated antibodies.

FIG. 12 panel B shows Western blots of passaged MEFs infected with the indicated constructs that express high levels of p53 and its target p21^{CIP1}. Western blots of cell lysates harvested from passaged cells were probed with the indicated antibodies.

FIG. 12 panel C is a line graph of MEFs infected with the indicated constructs, that were passaged in culture. Cumulative numbers of cells at each passage are shown on the ordinate.

FIG. 12 panel D shows Western blots of passaged cells infected with the indicated constructs were probed with the indicated antibodies.

FIG. 13 is a set of photographs of Western blots showing that the induction of p21^{CIP1} by Ndy1 in HCT116 cells is p53-dependent. P53−/− HCT116 cells and derivatives of these cells engineered to express p53 were infected with pBabe-puro or pBabe-Ndy1 constructs. The expression of p21^{CIP1} and PARP (control) were examined by Western blotting before and after treatment with Adriamycin (0.5 μM for 16 hours).

FIG. 14 is a set of bar graphs and photographs that show Ndy1 represses the senescence-associated upregulation of p16^{Ink4a} and p19^{Arf}.

FIG. 14 panel A shows total mRNA isolated from MEFs at the indicated passage and analyzed by real time PCR for the relative mRNA levels of Bmi1, Ezh2, Suz12, and Eed, Ndy1, and Ndy2 (n=2), *p<0.05.

FIG. 14 panel B is a set of bar graphs of MEFs overexpressing Ndy1 that were plated at a concentration of 105 cells per 6 cm dish and serially passaged. In passage P5, P10, and P15 total mRNA was isolated and analyzed by real time PCR for the relative mRNA levels of p16^{Ink4a} and p19^{Arf}. The graphs show the fold decrease of p16^{Ink4a} and p19^{Arf} mRNAs in MEFs that overexpress Ndy1 normalized to control empty-vector transduced cells from 3 independent infections.

FIG. 14 panel C is a set of photographs of Western blots of MEFs as in (B) that were serially passaged and whole cell lysates from the indicated passages, analyzed by Western blotting with the indicated antibodies. p19^{Arf} was undetectable at passages 2 and 5.

FIG. 14 panel D shows passage 3 MEFs that were transfected with siRNA that specifically targets either the long or the short form of Ndy1. At a time point four days after transfection cells were analyzed by Western blotting and total mRNA was collected and analyzed by real-time PCR with primers specific for p16^{Ink4a} and p19^{Arf} and primers specific for the long and short forms of Ndy1. (n=3)*p<0.05

FIG. 15 is a bar graph showing relative mRNA levels of different demethylases in early and late passage MEFs.

Total mRNA was isolated from MEFs at the indicated passages and analyzed by real time PCR for the relative mRNA levels of the indicated demethylases.

FIG. 16 is a line graph showing Ndy1 represses the senescence-associated upregulation of p16^{Ink4a} in MEFs.

The graph shows the fold induction of p16^{Ink4a} in empty vector and Ndy1 transduced MEFS, on the ordinate, at passages 2, 5, 10, and 15, on the abscissa.

FIG. 17 is a set of photographs and bar graphs that show that Ndy1 counteracts the senescence associated downregulation of Ezh2 and induces global H3K27 tri-methylation.

FIG. 17 panel A shows the same samples described in FIG. 1 panel C that were further analyzed by Western blotting with the indicated antibodies.

FIG. 17 panel B shows passage 3 MEFs that were transfected with an siRNA specific for Ndy1. Four days after transfection cells were collected and analyzed by Western blotting. The graph in the bottom of the figure shows the efficiency of Ndy1 knock down. The asterisk (*) indicates a non-specific band.

FIG. 17 panel C shows Western blotting analysis of whole cell extracts from MEFs immortalized with the MigR1-Ndy1 LoxP retroviral construct before and after infection with a retroviral construct that expresses the Cre recombinase.

FIG. 17 panel D shows the fold difference of different mRNAs in MigR1-Ndy1 LoxP immortalized MEFs before and after infection with the Cre recombinase (n=2).

FIG. 17 panel E shows chromatin immunoprecipitation (ChIP) analysis of the tri-methylation status of H3K27 at the Ink4a/Arf locus in early and late passage MEFs that overexpress Ndy1. The graph shows the fold increase of H3K27me3 methylation at the Ink4/Arf locus in MEFs that overexpress Ndy1, normalized to amount in control empty-vector transduced cells. In FIGS. 2D and 2E MEFs were immortalized with a Mig-R1-based retrovirus construct of Ndy1 flanked by two Lox P sites (FIG. 27).

FIG. 17 panel F shows ChIP analysis of Bmi1 binding at the Ink4a/Arf locus in passage 3-5 MEFs that overexpress Ndy1. The graph shows the fold increase of Bmi1 binding at the Ink4a/Arf locus in MEFs that overexpress Ndy1 normalized to control empty-vector transduced cells. *p<0.05 and # p<0.01.

FIG. 18 is a drawing showing a graphical representation of the MigR1-Ndy1 Lox P retroviral construct. LTR, long terminal repeat; IRES, internal ribosomal entry site; GFP/RFP, green/red fluorescence protein. Not to scale.

FIG. 19 is a set of photographs and bar graphs that show that Ndy1 cooperates with Ezh2 and Bmi1 to repress the Ink4/Arf locus.

FIG. 19 panel A shows passage 2 MEFs that were transfected with the indicated siRNA for 4 days. Whole cells lysates were analyzed by Western blotting with the indicated antibodies.

FIG. 19 panel B shows Ndy1 transduced MEFs that were transfected with siRNA specific for Bmi1. Whole cells lysates were analyzed by Western blotting with the indicated antibodies.

FIG. 19 panel C shows ChIP analysis of Ndy1 binding at the Ink4a/Arf locus in MEFs. The graph shows the fold enrichment of Myc tagged Ndy1 at the Ink4a/Arf locus normalized to control empty-vector transduced cells. p<0.05 and # p<0.01.

FIG. 19 panel D shows HEK293T cells that were transfected with the empty vector and Myc-tagged Ndy1. Whole cell lysates were immunoprecipitated with the Myc antibody and probed with an antibody specific for Ezh2 (top panel) and a mix of antibodies specific for Myc and tubulin (bottom panel).

FIG. 20 is a set of photographs and bar graphs that show that Ndy1 functions in vitro and in vivo as H3K36me2 and H3K4me3 demethylase.

FIG. 20 panel A shows representative Western blot analysis of an in vitro demethylation assay with native murine Ndy1 protein isolated from MEFs overexpressing Ndy1.

FIG. 20 panel B shows fluorescence coupled demethylation assay of bacterial purified fragments of Ndy1 using the indicated peptide substrates.

FIG. 20 panel C shows ChIP analysis of the H3K36me2 methylation status at the Ink4a/Arf locus in MEFs that overexpress Ndy1. The graph shows the fold decrease of H3K36me2 methylation at the Ink4a/Arf locus in MEFs that overexpress Ndy1 normalized to control empty-vector transduced cells.

FIG. 20 panel D shows ChIP analysis of the H3K4me3 methylation status at the Ink4a/Arf locus in MEFs that overexpress Ndy1. The graph shows the fold difference of H3K4me3 methylation at the Ink4a/Arf locus in MEFs that overexpress Ndy1 normalized to control empty-vector transduced cells.

FIG. 20 panel E shows ChIP analysis of RNA Pol II binding at the Ink4a/Arf locus in MEFs that overexpress Ndy1. The graph shows the fold difference of RNA Pol II binding at the Ink4a/Arf locus in MEFs that overexpress Ndy1 normalized to control empty-vector transduced cells. *p<0.05 and # p<0.01.

FIG. 21 is a set of photographs showing the human Ndy1 exhibits in vitro H3K36me2 and H3K4me3 demethylase activities.

Representative Western blot analyses of an in vitro demethylation assay were performed with native human Ndy1 protein isolated from IMR90 overexpressing human Ndy1.

FIG. 22 is a table showing that Ndy1 exhibits about three times higher specific activity towards the H3K36me2 versus the H3K4me3 peptide.

Cumulative data show the specific activity of different Ndy1 fragments (expressed in E. coli and purified from bacterial extracts) towards the H3K36me2 and H3K4me3 peptides.

FIG. 23 is a set of line graphs showing that Ndy1 overexpression reduces the global levels of H3K36me2 methylation.

Flow cytometric analysis was performed of HEK293 cells overexpressing Ndy1 and probed with antibodies that recognize the indicated histone modifications. The right panel is a bar graph that shows the quantification of the mean fluorescence intensity for the indicated histone modifications in empty vector and Ndy1 transduced HEK293 cells.

FIG. 24 is a set of graphs and photographs that show that Ndy1/KDM2B overexpression protects MEFs from Ras-induced senescence and represses the passage-dependent and Ras-induced upregulation of p16^{Ink4a} and p19^{Arf}.

FIG. 24 panel A is a wet of line graphs showing MEFs transduced with RasV12 alone or along with Ndy1/KDM2B or Bmi1 that were plated under two different concentrations (2×10⁴ or 10⁴ cells/well upper and low panel, respectively) in duplicate in a 12-well plate format. Cells were counted every 3 days and replated at the same concentrations. The graph shows the cumulative number of cells counted passage by passage.

FIG. 24 panel B is a set of photographs of MEFs overexpressing oncogenic Ras alone or along with Ndy1/KDM2B or Bmi1 that were plated at a concentration of 2×10⁴ cells per 10 cm dish. Cells were cultured for 3 weeks and colonies were visualized after crystal violet staining.

FIG. 24 panel C is a set of bar graphs of mRNA isolated from passage 3 MEFs overexpressing RasV12 alone or along with Ndy1/KDM2B or Bmi1 was analyzed by real time-PCR for the relative levels of p16^{Ink4a} (left) and p19^{Arf} (right) mRNAs. The graphs show the fold difference of p16^{Ink4a} and p19^{Arf} mRNAs in MEFs that overexpress oncogenic Ras alone or along with Ndy1/KDM2B or Bmi1 normalized to control empty-vector infected cells from 2 independent infections. *p<0.05.

FIG. 24 panel D shows the same cells as in panel C that were analyzed by Western blotting with the indicated antibodies.

FIG. 25 is a set of photographs and graphs with error bars showing that Ndy1 upregulates Ezh2 and represses p16^{Ink4a} in IMR90 cells.

FIG. 25 panel A shows total mRNA that was isolated from Ndy1 or empty-vector transduced IMR90 cells at passage 20 and analyzed by real time PCR for the relative mRNA levels of p16^{Ink4a}, n=2, *p<0.05.

FIG. 25 panel B shows Ndy1 or empty-vector transduced IMR90 cells that were serially passaged and analyzed by Western blotting.

FIG. 25 panel C shows data from (37) reanalyzed to show expression levels of Ndy1 in normal bone marrow, B- and T-cell acute lymphoblastic leukemias, and acute myeloid leukemia.

FIG. 25 panel D shows data from (38) reanalyzed to show expression levels of Ndy1 in normal testis and seminomas.

FIG. 26 is a bar graph showing that Ndy1 overexpression increases the mRNA levels of Ezh2 in IMR90 cells.

Total mRNA was isolated from Ndy1 or empty-vector transduced IMR90 cells at passage 20 and analyzed by real time PCR for the relative mRNA levels of the indicated mRNAs, n=2.

FIG. 27 is a bar graph showing that Ndy1 overexpression increases the mRNA levels of Ezh2 in MEFs.

Total mRNA from MEFs transduced either with Ndy1 or empty vector was analyzed by real time PCR for the relative levels of Ezh2 mRNA. (n=4), *p<0.05.

FIG. 28 is a drawing showing a model of the histone demethylase Ndy1 repressing the Ink4a/Arf locus. Ndy1 represses the Ink4a/Arf locus by two distinct mechanisms: it upregulates Ezh2 and promotes histone H3K27 tri-methylation and Bmi1 binding within the Ink4a/Arf locus; and, it binds to and demethylates H3K36me2 and H3K4me3 within the Ink4a/Arf locus. These histone modifications combined, interfere with the binding of RNA Pol II and contribute to the silencing of the Ink4a/Arf locus.

FIG. 29 is a bar graph and a set of photographs comparing the expression of Ndy1 in total bone marrow (BM) and isolated hematopoietic stem cells (HSCs), showing that Ndy1 is expressed at significantly higher levels in the HSCs.

FIG. 29 panel A shows a bar graph with results of real time RT-PCR assays using BM and HSC, with expression of NDY1 over an order of magnitude greater in the HSC. Western blot of four cell types show that the two stem cell lines (ES and HSC) produce more than total BM or mouse embryo fibroblasts (MEF). Actin production was measured by Western blot as a control, and is equivalent in the four cell types.

FIG. 29 panel B shows a Western blot of NDY1 production as a function of time during growth of ES cells absent a fibroblast feeder layer and without LIF, showing that as cells differentiate into fibroblast-like cells that Hdy1 expression declines. Actin production was measured by Western blot as a control, and remained at the same level during the time course.

FIG. 30 is a bar graph and a flow cytometric analysis showing that total bone marrow cells infected with a vector carrying Ndy1 continue to grow during serial passaging.

FIG. 30 panel A is a bar graph comparing each of three lines of cells: those infected with the Ndy1 vector, those infected by an Ndy1 mutant, and those infected by the empty vector control. After three and four passages, only the Ndy1 containing cells grew substantially. By passage 4, growth of the other two cell lines was negligible.

FIG. 30 panel B shows that cells infected with Ndy1 vector expressed each of c-Kit and Seal at a level indistinguishable from bone marrow cells grown in semi-solid medium.

FIG. 31 is a photograph, a line graph, and light scattering and flow cytometric analyses of cells in which Ndy1 gene expression is extinguishable due to deletion by the Cre-Lox system.

FIG. 31 panel A is a photograph of a Western blot showing deletion of Ndy1 expression in the presence of both Cre and Lox eliminates expression, compared to control actin the expression of which was not affected.

FIG. 31 panel B is a light scattering analysis showing that following deletion of Ndy1 by Cre-Lox the cells become larger and more granular.

FIG. 31 panel C is a line graph showing cell number as a function of days of culture, and diminished growth in the presence of Cre.

FIG. 31 panels D and E are flow cytometric analyses of cells superinfected with Cre virus showing that some of the cells express antigens B220 and CD44 (panel E) compared to absence of Cre (panel D), indicating differentiation under the culture conditions.

FIG. 32 is a set of photographs, bar graphs and drawings showing that overexpression of Ndy1 downregulates miR-101 expression and upregulates Ezh2 protein levels.

FIG. 32 panel A shows that overexpression of Ndy1 upregulates Ezh2 at the protein rather than at the mRNA level. MEFs were transduced with an HA.Ndy1 overexpression construct, or the empty vector (EV). (left) Nuclear extracts were probed with antibodies specific for each of HA-tag (Ndy1) and Ezh2. CREB levels served as loading control. (right) Ezh2 mRNA levels were measured with real time RT-PCR; GAPDH mRNA levels were used as internal control. Data are expressed as mean±SD.

FIG. 32 panel B shows that overexpression of Ndy1 downregulates miR-101 expression. (right) miR-101 levels were measured with real time RT-PCR; U6 small nuclear RNA levels were used as internal control. (left) The product of the real time RT-PCR reaction visualized with ethidium bromide staining in 2% agarose gel. Data are expressed as mean±SD.

FIG. 32 panel C shows that excision of the exogenous Ndy1 gene reverses the effect of Ndy1 overexpression. MEFs were transduced with a loxp.myc.Ndy1.loxp overexpression construct and Ndy1 was subsequently excised by transduction with Cre recombinase. (left) extracts were probed with antibodies specific for each of myc-tag (Ndy1) and Ezh2. and anti-CREB antibody was used as loading control. (middle) miR-101 levels were measured with real time RT-PCR; U6 small nuclear RNA levels were used as internal control. Data are expressed as mean±SD. (right) The engineered cells were transfected with a miR-101 promoter-luciferase reporter construct. Relative luciferase activity was calculated as the ratio of firefly to Renilla luciferase activity and was set as 1 in Ndy1-overexpressing cells. Data are expressed as mean±SD.

FIG. 32 panel D shows that human and mouse Ezh2 3′ UTR share conserved regions for binding of miR-101. Graphical representation of human and mouse Ezh2 3′ UTR and the miR-101 binding site in sequences of each organism (gray). The sequences shown are:

| mouse: | |
|---|---|
| CUUCAGGAACCUUGAGUACUGUG, | (SEQ ID NO: 82) |
| human: | |
| CUUCAGGAACCUCGAGUACUGUG, | (SEQ ID NO: 83) |
| and | |
| for both species: | |
| UUCUGAAUUUGCAAAGUACUGUA. | (SEQ ID NO: 84) |

FIG. 32 panel E shows that miR-101 represses Ezh2 in the presence of Ndy1. MEFs were transduced with an HA.Ndy1 overexpression vector, or the empty vector (EV) and were transfected with pre-miR-101. Nuclear extracts were probed with antibodies specific for each of HA-tag (Ndy1) and Ezh2. CREB levels served as loading control.

FIG. 33 is a graphical representation of the Luciferase reporter construct with the miR-101 promoter. The 1 kb region of miR-101 (dark bar) was cloned in the PGL3 basic vector, followed by the firefly luciferase gene (luc+).

FIG. 34 is a set of photographs and bar graphs showing effect of Ndy1 alterations on serum stimulated MEFs.

FIG. 34 panel A shows that serum stimulation upregulates both Ndy1 and Ezh2 mRNAs. MEFs were transfected with control siRNA and stimulated with serum (10%) for 1, 4, 12 and 18 hours. (upper) Nuclear extracts were probed with antibody specific for Ezh2 and for CREB, levels of which served as loading control. (lower) Ndy1 and Ezh2 mRNA levels were measured with real time RT-PCR; GAPDH mRNA levels were used as internal control. Data are expressed as mean±SD.

FIG. 34 panel B shows that Ndy1 knockdown does not affect serum-stimulated increase in Ezh2 mRNA levels. MEFs were transfected with siRNA for Ndy1 and stimulated with serum (10%) for 1, 4, 12 and 18 hours. (upper) Nuclear extracts were probed with antibodies specific for Ezh2 and for CREB, levels of which served as loading control. (lower) Ndy1 and Ezh2 mRNA levels were measured with real time RT-PCR; GAPDH mRNA levels were used as internal control. Data are expressed as mean±SD.

FIG. 35 is a set of photographs and bar graphs showing that FGF-2 stimulation activates the Ndy1-miR-101-Ezh2 axis.

FIG. 35 panel A shows that Ndy1 and Ezh2 protein and mRNA levels decreased and miR-101 levels remained unchanged during passaging. MEFs were passaged every three days, plating 1.5×10⁶ cells into a 10 cm plate each passage. Nuclear extracts from the respective passages were probed with antibodies specific for Ezh2 and for CREB, which was used as loading control.

FIG. 35 panel B shows Ndy1 and Ezh2 mRNAs and miR-101 levels from the respective passages, measured with real time RT-PCR; GAPDH mRNA and U6 small nuclear RNA levels were used as internal controls. Data are expressed as mean±SD.

FIG. 35 panel C shows that knocking down Ndy1 had no effect on levels of miR-101. MEFs were transfected with either a control siRNA or siRNA for Ndy1. Nuclear extracts were probed with antibody specific for Ezh2 and for CREB, which served as loading control.

FIG. 35 panel D shows Ndy1 mRNA and miR-101 levels which were measured with real time RT-PCR; GAPDH mRNA and U6 small nuclear RNA levels were used as internal controls. Data are expressed as mean±SD.

FIG. 35 panel E shows that FGF-2 and VEGF activate the Ndy1-miR-10′-Ezh2 axis. Serum starved MEFs were stimulated with each of FGF-2 (20 ng/ml), IGF-1 (20 ng/ml), PDGF (20 ng/ml), serum (10%), VEGF (20 ng/ml), EGF (20 ng/ml) or TNFα (50 ng/ml). Nuclear extracts were probed with antibody specific for Ezh2 and for CREB, levels of which served as loading control.

FIG. 35 panel F shows Ndy1 and Ezh2 mRNA and miR-101 levels, which were measured with real time RT-PCR; GAPDH mRNA and U6 small nuclear RNA levels were used as internal controls. Data are expressed as mean±SD.

FIG. 35 panel G shows that oxidative stress has no effect on Ezh2 protein levels. MEFs were treated with NAC (5 mM), Trolox (1 mM) or H₂O₂ (0.5 or 1 mM). Nuclear extracts were probed with antibodies specific for Ezh2 and for CREB, levels of which served as loading control.

FIG. 36 is a set of photographs and bar graphs showing that Ndy1 mediates the FGF-2-induced Ezh2 upregulation by repressing miR-101 expression.

FIG. 36 panel A shows that stimulation by FGF-2 upregulates Ndy1, down-regulates miR-101 and upregulates Ezh2. MEFs transfected with control siRNA were stimulated with FGF-2 for 1, 4, 12 or 18 hours. (upper) Nuclear extracts were probed with antibody specific for Ezh2. CREB levels served as loading control. (middle) Ndy1 and Ezh2 mRNA were measured with real time RT-PCR; GAPDH mRNA levels were used as internal control. Data are expressed as mean±SD. (lower) miR-101 levels were measured with real time RT-PCR; U6 small nuclear RNA levels were used as internal control. Data are expressed as mean±SD.

FIG. 36 panel B shows the effect of Ndy1 knockdown on Ezh2 levels on FGF-2 stimulation. MEFs transfected with siRNA for Ndy1 were stimulated with FGF-2 for 1, 4, 12 or 18 hours. (upper) Nuclear extracts were probed with antibody specific for Ezh2. CREB levels served as loading control. (middle) Ndy1 and Ezh2 mRNA were measured with real time RT-PCR; GAPDH mRNA levels were used as internal control. Data are expressed as mean±SD. (lower) miR-101 levels were measured with real time RT-PCR; U6 small nuclear RNA levels were used as internal control. Data are expressed as mean±SD.

FIG. 37 is a set of photographs and bar graphs showing that DNA-binding properties and demethylase activity of Ndy1 are essential for miR-101 repression. MEFs were transduced with HA.Ndy1 (wild type), HA.ΔCxxC (deletion Ndy1 mutant lacking the CxxC domain), HA.ΔFbox (deletion Ndy1 mutant lacking the Fbox domain) and HA.Ndy1 (H283Y) (point mutant of Ndy1 in the Jmjc domain, demethylase dead mutant) overexpression constructs, or the empty vector (EV).

FIG. 37 panel A shows nuclear extracts that were probed with antibodies specific for each of HA-tag and Ezh2 which were observed to be overexpressed in some of these cell lines. CREB levels served as loading control.

FIG. 37 panel B shows miR-101 levels measured with real time RT-PCR; U6 small nuclear RNA levels were used as internal control. Data are expressed as mean±SD.

FIG. 37 panel C shows engineered cells that were transfected with a miR-101 promoter-luciferase reporter construct. Relative luciferase activity was calculated as the ratio of firefly over renilla luciferase activity and was set as 1 in the EV cells. Data are expressed as mean±SD.

FIG. 38 is a set of bar graphs showing that Ndy1 binds to the miR-101 locus, and recruits Ezh2 and Bmi1, leading to dissociation of RNA polymerase II and silencing of miR-101 expression.

FIG. 38 panel A is a graphical representation of the miR-101 (shaded box) genomic locus and relative position of the primers used in the chromatin immunoprecipitation analyses. MEFs were transduced with an HA.Ndy1 overexpression construct, or the empty vector (EV) and subjected to chromatin immunoprecipitation with each of the following antibodies: anti-HA (Ndy1), anti-dimethyl-H3K36, anti-Ezh2, anti-trimethyl-H3K27, anti-trimethyl-H3K4, anti-Bmi1 and anti-RNA polymerase II. The fold enrichment of FIG. 38 panel B HA.Ndy1, FIG. 38 panel C H3K36 dimethylation, FIG. 38 panel D Ezh2, FIG. 38 panel E H3K27 trimethylation, FIG. 38 panel F H3K4 trimethylation, FIG. 38 panel G Bmi1, and FIG. 38 panel H RNA polymerase II to the promoter of miR-101, was analyzed by real time PCR. Data are expressed as mean±SD.

FIG. 39 is a set of line graphs, bar graphs and photographs showing that FGF-2-induced proliferation and migration are mediated by suppression of miR-101 by Ndy1.

FIG. 39 panel A shows that FGF-2 induced proliferation and migration is inhibited by Ndy1 knockdown. MEFs were transfected with each of: control RNA, pre-miR-101, siRNA for Ndy1 and stimulated with FGF-2 or carrier negative control. Cell number was quantified after 24, 48 and 72 hours of growth with or without FGF-2. Data are expressed as mean±SD.

FIG. 39 panel B shows FGF-2-induced directional migration of the cells. Migration was measured with the transwell filter assay. Relative migration was calculated as the ratio of the number of migrating cells transfected with each of control RNA, siRNA for Ndy1 or pre-miR-101 under stimulation by FGF-2, greater than the number of migrating cells transfected with control RNA, set to 100 for the number of migrating cells transfected with control RNA. Data are expressed as mean±SD.

FIG. 39 panel C shows nuclear extracts that were probed with antibody specific for Ezh2. CREB levels served as loading control.

FIG. 39 panel D shows Ndy1 mRNA and miR-101 that were measured with real time RT-PCR; GAPDH and U6 small nuclear RNA, respectively, were used as internal controls (48 hours under FGF-2 stimulation).

FIG. 39 panel E shows wound healing assays that were carried out on a monolayer of confluent cultures, of the same MEFs in panel A. The images show the wounded areas at 12 h after wounding.

FIG. 39 panel F shows that Ndy1 overexpression increases proliferation and motility in the absence of FGF-2. MEFs were transduced with an HA.Ndy1 overexpression construct, or the empty vector (EV) and were subsequently transfected with control miRNA or with pre-mir-101. Cell number was quantified after 24, 48 and 72 hours of growth without FGF-2. Data are expressed as mean±SD.

FIG. 39 panel G shows migration of the same cells as in panel F, measured with the transwell filter assay. Relative migration was calculated as the ratio of the number of migrating cells transfected with control miRNA or pre-miR-101 greater than the number of migrating cells transfected with control miRNA, set to 100 for the EV cells transfected with control miRNA. Data are expressed as mean±SD.

FIG. 39 panel H shows that in FGF-2 treated cells, overexpression of Ezh2 rescues the Ndy1-knockdown phenotype. MEFs were transduced with a myc.Ezh2 overexpression construct, or the empty vector (EV) and were subsequently transfected with each of control RNA, siRNA for Ndy1 or pre-mir-101. Cell number was quantified after 24, 48 and 72 hours of growth with FGF-2. Data are expressed as mean±SD.

FIG. 39 panel I shows migration of the same cells as in panel H, measured with the transwell filter assay. Relative migration was calculated as the ratio of the number of migrating cells transfected with each of control RNA, siRNA for Ndy1 or pre-miR-101 greater than the number of migrating cells transfected with control RNA, set to 100 for the number of migrating EV cells transfected with control RNA. Data are expressed as mean±SD.

FIG. 40 is a set of line graphs showing that Ndy1, miR-101 and Ezh2 alterations affect FGF-2-induced wound healing.

FIG. 40 panel A shows MEFs that were transfected with each of control RNA, siRNA for Ndy1 or miR-101 and were treated with FGF-2 (20 ng/ml) (right) or carrier (left). Wound healing assays were carried out on a monolayer of confluent cultures. Relative migration was calculated as the ratio of distance covered by cells over total distance between the edges of the wound and was set to 100 for the total distance between the edges of the wound. Ndy1 alterations in cells engineered to overexpress Ezh2 were observed to have no effect on FGF-2-induced migration (A). MEFs transduced with a myc.Ezh2 overexpression construct (FIG. 40 panel B), or the empty vector (EV), were subsequently transfected with control RNA, siRNA for Ndy1 or miR-101 and were treated with FGF-2 (20 ng/ml) (right) or carrier. Wound healing assays were carried out on a monolayer of confluent cultures. Relative migration was calculated as the ratio of distance covered by cells over total distance between the edges of the wound and was set to 100 for the total distance between the edges of the wound. FIG. 40 panel B shows results using empty vector (EV) cells, and FIG. 40 panel C shows results using Ezh2 cells.

FIG. 41 is a set of bar graphs and photographs showing in vitro FGF-2-induced tube formation mediated by Ndy1.

FIG. 41 panel A shows the Ndy1-miR-101-Ezh2 axis in Human Umbilical cord Vein Endothelial Cells (HUVECs) transduced with shRNA for Ndy1 or with a lentiviral-based control shRNA. (left) Nuclear extracts were probed with antibodies specific for Ndy1 and Ezh2. CREB levels served as loading control. (right) miR-101 levels we measured with real time RT-PCR, in the same cells; U6 small nuclear RNA levels were used as internal control. Data are expressed as mean±SD.

FIG. 41 panel B shows effect of Ndy1 in FGF-2-induced tube formation of HUVECs. HUVECs were transduced with a myc.Ezh2 expression construct, or with the empty vector (EV) and were subsequently transduced with either shRNA for Ndy1 or with a lentiviral-based control shRNA. (upper-first row) Tube formation on Cultrex-RGF-BME without FGF-2. (upper-second row) Tube formation on Cultrex-RGF-BME in the presence of 20 ng/ml FGF-2. (upper-third row) Tube formation on reduced-growth factor-Matrigel in the presence of 5 μM of the angiogenesis inhibitor sulphoraphane. (lower) Quantitation of tube formation was performed with the Angioquant software. Relative tube formation was calculated as the ratio of total capillary tube length in cells with or without FGF-2 normalized to total capillary tube length of EV cells without FGF-2 and set to 1 for total capillary tube length of EV cells without FGF-2. Data are expressed as mean±SD.

FIG. 42 is a set of photographs and bar graphs showing that the Ndy1-miR-101-Ezh2 axis is active in cancer cell lines expressing high levels of FGF-2.

FIG. 42 panel A shows TCCSUP cells that were transfected with either the control siRNA or siRNA for Ndy1. (left) Nuclear extracts were probed with antibodies specific for each of Ndy1 and Ezh2. CREB levels served as loading control. (right) miR-101 levels were measured with real time RT-PCR; U6 small nuclear RNA levels were used as internal control. Data are expressed as mean±SD.

FIG. 42 panel B shows that treatment with FGF receptor inhibitor PD173074 downregulates Ndy1 and Ezh2 and upregulates miR-101 levels in tumor cells. TCCSUP cells were treated with 0.5 μM PD173074 or carrier for 24 hours. (upper) Nuclear and cytoplasmic extracts were probed with antibodies specific for each of Ndy1, Ezh2 and phosphoERK. CREB and ERK levels served as loading controls. (lower) miR-101 levels were measured with real time RT-PCR; U6 small nuclear RNA levels were used as internal control. Data are expressed as mean±SD.

FIG. 42 panel C shows that PD173074 effect on miR-101 and Ezh2 is abrogated with overexpression of Ndy1. TCCSUP cells were transduced with an HA.Ndy1 overexpression construct or with the empty vector (EV), and were subsequently treated with 0.5 μM PD173074 or carrier for 24 hours. (upper) Nuclear extracts were probed with antibodies specific for each of HA-tag (exogenous Ndy1), Ndy1 (both endogenous and exogenous Ndy1) and Ezh2. CREB levels served as loading control. (lower) miR-101 levels were measured with real time RT-PCR; U6 small nuclear RNA levels were used as internal control. Data are expressed as mean±SD.

FIG. 42 panel D shows WIDR cells that were transfected with either the control siRNA or siRNA for Ndy1. (left) Nuclear extracts were probed with antibodies specific for each of Ndy1 and Ezh2. CREB levels served as loading control. (right) miR-101 levels were measured with real time RT-PCR; U6 small nuclear RNA levels were used as internal control. Data are expressed as mean SD.

FIG. 42 panel E shows that PD173074 treatment downregulates Ndy1 and Ezh2 and upregulates miR-101 levels. WIDR cell were treated with 0.5 μM PD173074 or carrier for 24 hours. (upper) Nuclear and cytoplasmic extracts were probed with antibodies specific for each of Ndy1, Ezh2 and phosphoERK antibodies. CREB and ERK levels served as loading controls. (lower) miR-101 levels were measured with real time RT-PCR; U6 small nuclear RNA levels were used as internal control. Data are expressed as mean±SD.

FIG. 42 panel F shows that PD173074 effect on miR-101 and Ezh2 is abrogated with overexpression of Ndy1. WIDR cells were transduced with an HA.Ndy1 overexpression construct or the empty vector (EV), and were subsequently treated with 0.5 μM PD173074 or carrier for 24 hours. (upper) Nuclear extracts were probed with antibodies specific for each of with HA-tag (exogenous Ndy1), Ndy1 (both endogenous and exogenous Ndy1) and Ezh2. CREB levels served as loading controls. (lower) miR-101 levels were measured with real time RT-PCR; U6 small nuclear RNA levels were used as internal controls. Data are expressed as mean±SD.

FIG. 42 panel G shows FGF-2 mRNA levels that were measured with real time RT-PCR, GAPDH mRNA levels were used as control.

FIG. 43 is a set of photographs and bar graphs showing that the Ndy1-miR-101-Ezh2 axis is active in cancer cell lines expressing high levels of FGF-2.

FIG. 43 panel A shows data obtained from SKOV3 cells that were transfected with either the control siRNA or siRNA for Ndy1. (upper) Nuclear extracts were probed with antibodies specific for each of Ndy1 and Ezh2. CREB levels served as loading control. (lower) miR-101 levels were measured with real time RT-PCR; U6 small nuclear RNA levels were used as internal control. Data are expressed as mean±SD.

FIG. 43 panel B shows data obtained from NCI522 cells that were transfected with either the control siRNA or siRNA for Ndy1. (upper) Nuclear extracts were probed with antibodies specific for each of Ndy1 and Ezh2. CREB levels served as loading control. (lower) miR-101 levels were measured with real time RT-PCR; U6 small nuclear RNA levels were used as internal control. Data are expressed as mean±SD.

FIG. 43 panel C shows data obtained from T24 cells that were transfected with either the control siRNA or siRNA for Ndy1. (upper) Nuclear extracts were probed with antibodies specific for each of Ndy1 and Ezh2. CREB levels served as loading control. (lower) miR-101 levels were measured with real time RT-PCR; U6 small nuclear RNA levels were used as internal control. Data are expressed as mean±SD.

FIG. 44 is a set of photographs and bar graphs showing that the Ndy1-miR-101-Ezh2 axis operates during oncogenesis.

FIG. 44 panel A shows that Ndy1 is a common target of provirus integration in MoMuLV-induced rat T-cell lymphomas. (upper) Total cell extracts from normal thymus or the indicated tumors were probed with antibodies specific for each of Ezh2, and α-Tubulin was used as loading control. (middle and lower) Ndy1 mRNA and miR-101 levels were measured with real time RT-PCR; GAPDH mRNA and U6 small nuclear RNA levels, respectively, were used as internal controls.

FIG. 44 panel B shows that Ndy1 is expressed in higher level in bladder carcinomas than in normal bladder tissue, and that Ndy1 correlates with decreased miR-101 expression and increased Ezh2 protein levels. Representative images were taken from a 48-core (40 carcinomas and eight of normal tissue) bladder carcinoma tissue array. (left column) In situ hybridization for Ndy1 (upper), miR-101 (middle), and immunohistochemical detection of Ezh2 (lower) of a representative normal tissue. (middle column) In situ hybridization for Ndy1 (upper) and miR-101 (middle) and immunohistochemical detection of Ezh2 (lower) of a representative bladder carcinoma with low levels of Ndy1. (right column) in situ hybridization for Ndy1 (upper) and miR-101 (middle) and immunohistochemical detection of Ezh2 (lower) of a representative bladder carcinoma with high levels of Ndy1.

FIG. 44 panel C shows a non-linear inverse correlation between Ndy1 and miR-101 expression. Pearson's correlation analysis was used (correlation coefficient: −0.37, significance: 0.018).

FIG. 44 panel D shows a quantitation of in situ hybridization for Ndy1 (left) and miR-101 (middle) and immunohistochemical detection of Ezh2 (right), of normal tissues compared to carcinomas. The relative levels were calculated as the ratio of sample normalized to the mean value of normal tissue samples and were set to 1 for mean of nomad tissue samples. Significance was calculated using unpaired student's test.

FIG. 44 panel E shows a quantitation of in situ hybridization for Ndy1 (left) and miR-101 (middle) and immunohistochemical detection of Ezh2 (right), of carcinomas expressing high levels of Ndy1 versus carcinomas expressing low levels of Ndy1. The relative levels were calculated as the ratio of sample normalized to the mean value of normal tissue samples and were set to 1 for mean of normal tissue samples. Significance was calculated using unpaired student's test.

FIG. 45 is a bar graph showing chromatin immunoprecipitation data. MEFs were transduced with an HA.Ndy1 overexpression construct, or with the empty vector (EV), and were subjected to chromatin immunoprecipation with each of: anti-HA (Ndy1), anti-dimethyl-H3K36, anti-Ezh2, anti-trimethyl-H3K27, anti-trimethyl-H3K4, anti-Bmi1 and anti-RNA polymerase II. The fold differences of HA.Ndy1, H3K36 dimethylation, Ezh2, H3K27 trimethylation, H3K4 trimethylation, Bmi1 and RNA polymerase II to the promoter of prdx2 gene were determined by real time PCR. The prdx2 promoter was used as a negative control since Ndy1 does not bind to it. Data are expressed as mean±SD.

FIG. 46 is a set of bar graphs and photomicrographs of analyses of three independent lung cancer specimens for expression of Ndy1, Ezh2, and miR-101.

FIG. 46 panel A is a control to show that the probe used for Ndy1 is specific. 293T cells were transduced with a lentiviral-based shRNA for Ndy1 or with a control shRNA and were probed with an Ndy1 RNA probe. Data show visible stained cells with the Ndy1 transduced cells, and not with cells transduced with shRNA.

FIG. 46 panels B, C and D show analyses of three lung tumors from three different patients. FIG. 46 panel B shows Ndy1 and Ezh2 in the lung tumors. Total cell lysates were probed with antibodies specific for each of Ndy1 and Ezh2. α-Tubulin levels serve as loading control. Data show that tumor LC2 overexpresses both Ndy1 and Ezh2 compared to the tubulin loading control.

FIG. 46 panel C shows miR-101 levels in each of the three lung tumors, measured with real time RT-PCR; U6 small nuclear RNA levels were used as internal control. Data are expressed as mean±SD. Lung tumor LC2 expresses less than half the amount of miR-101 compared to the two other tumors.

FIG. 46 panel D shows cells stained for each of Ndy1 (top) and miR-101 (middle) by in situ hybridization, and for Ezh2 (bottom) by immunohistochemistry.

### DETAILED DESCRIPTION

Mutations caused by provirus integration into the genome play a critical role in the induction and progression of retrovirus-induced neoplasms (Tsichlis, P. N. et al. (1991) Curr Top Microbiol Immunol 171, 95-171; Gilks, C. B. et al. (1993) Mol Cell Biol 13, 1759-68; and Li, J. et al. (1999) Nat Genet 23, 348-53). Given that provirus integration is practically random, the detection of an integrated provirus within a given DNA region in multiple tumors indicates that the mutation caused by this provirus endows the affected cell with a selective advantage over its neighbors. Based on this understanding, the identification of common integration sites in retrovirus-induced tumors has been used as an effective tool to identify novel oncogenes.

A common integration site, cloned from MoMuLV-induced rat T cell lymphomas, was mapped immediately upstream of Not dead yet-1 (Ndy1), a gene expressed primarily in testis, spleen and thymus, that is also known as FBXL10 or JHDM1B. Ndy1 encodes a nuclear, chromatin-associated protein that harbors Jumonji C (JmjC), CXXC, PHD, proline-rich, F-box and leucine rich repeat domains. Ndy1 and its homolog Ndy2 (FBXL11 or JHDM1A), which is also a target of provirus integration in retrovirus-induced lymphomas, encode proteins that were recently shown to possess Jumonji C-dependent histone H3K36 dimethyl-demethylase or histone H3K4 trimethyldemethylase activities.

Mouse embryo fibroblasts were herein engineered to express Ndy1, or Ndy2, and were shown in examples herein to undergo immortalization in the absence of replicative senescence via a JmjC domain-dependent process that targets the Rb and p53 pathways. Knock down of endogenous Ndy1 or expression of JmjC domain mutants of Ndy1, were found herein to promote senescence, indicating that Ndy1 is a physiological inhibitor of senescence in dividing cells and that inhibition of senescence depends on histone H3 demethylation.

Evidence was obtained in examples herein that the two members of the JHDM1 protein family, Ndy1 (FBXL10 or JHDM1B) and Ndy2 (FBXL11 or JHDM1A), contribute to the induction and/or progression of MoMuLV-induced T cell lymphomas in rodents. Furthermore, upon overexpression, both proteins immortalize MEFs in culture. Moreover, knockdown of Ndy1 and expression of Ndy1 dominant negative mutants promote senescence, indicating that Ndy1 is a physiological inhibitor of senescence in dividing cells.

Immortalization was found herein to depend on the JmjC domain and perhaps on JmjC domain-mediated histone demethylation. Finally, the immortalization activity of Ndy1 was found to depend on targeting the Rb and p53 pathways.

The histone H3 demethylase Not dead yet-1 (Ndy1/KDM2B) is a physiological inhibitor of senescence. Here, we show that Ndy1 is downregulated during senescence in mouse embryonic fibroblasts (MEFs) and that it represses the Ink4a/Arf locus. Ndy1 counteracts the senescence-associated downregulation of Ezh2, a component of PRC2, via a JmjC domain-dependent process leading to the global and Ink4a/Arf locus-specific upregulation of histone H3K27 tri-methylation. The latter promotes the Ink4a/Arf locus-specific binding of Bmi1, a component of PRC1, which is known to repress the locus. Ndy1, which interacts with Ezh2, also binds the Ink4a/Arf locus and demethylates the locus-associated histone H3K36me2 and histone H3K4me3. The combination of histone modifications driven by Ndy1 interfere with the binding of RNA Polymerase II, resulting in the transcriptional silencing of the Ink4a/Arf locus and contributing to the Ndy1 immortalization phenotype. Other examples herein show that, in addition to inhibiting replicative senescence, Ndy1 also inhibits Ras oncogene-induced senescence via a similar molecular mechanism.

Cellular senescence is an irreversible growth arrest that is either developmentally programmed in dividing cells, or is triggered by several types of stress, such as DNA damage, telomere shortening, and oncogene activation (Collado, M. et al. (2007) Cell 130, 223-33; Gil, J. et al. (2006) Nat Rev Mol Cell Biol 7, 667-77). At the molecular level, senescence is characterized by the activation of the Ink4a/Arf locus which encodes two proteins, p16^{Ink4a} and p19^{Arf} (p14^{Arf} in humans; Collado, M. et al. (2007) Cell 130, 223-33; Gil, J. et al. (2006) Nat Rev Mol Cell Biol 7, 667-77; Sharpless, N. E., et al. (2001) Nature 413, 86-91; Serrano, M., et al. (1996) Cell 85, 27-37; Kamijo, T., et al. (1997) Cell 91, 649-59), that regulate the Rb and p53 pathways, respectively (Collado, M. et al. (2007) Cell 130, 223-33; Gil, J. et al. (2006) Nat Rev Mol Cell Biol 7, 667-77). p16Ink4a inhibits the cyclin-dependent kinases Cdk4 and Cdk6 that phosphorylate and inactivate Rb (Collado, M. et al. (2007) Cell 130, 223-33; Gil, J. et al. (2006) Nat Rev Mol Cell Biol 7, 667-77). p19^{Arf} interacts with the ubiquitin ligase MDM2 and inhibits MDM2-mediated p53 degradation (Collado, M. et al. (2007) Cell 130, 223-33; Gil, J. et al. (2006) Nat Rev Mol Cell Biol 7, 667-77). Overexpression of the Polycomb group (PcG) proteins Bmi1 (Jacobs, J. J., et al. (1999) Nature 397, 164-8), Ezh2 (Kamminga, L. M., et al. (2006) Blood 107, 2170-9), CBX7 (Gil, J., et al. (2004) Nat Cell Biol 6, 67-72), and CBX8 (Dietrich, N., et al. (2007) Embo J 26, 1637-48) delays the onset of replicative senescence in mouse and human embryonic fibroblasts by repressing the Ink4a/Arf locus.

The PcG proteins are involved in the maintenance of cell identity and stem cell renewal and contribute to cell cycle regulation and oncogenesis (Schwartz, Y. B., et al. (2007) Nat Rev Genet 8, 9-22 and Sparmann, A., et al. (2006) Nat Rev Cancer 6, 846-56). PcG proteins exist in two distinct complexes that cooperate to maintain long-term gene silencing through chromatin modifications. Polycomb-repressive complex 2 (PRC2) contains Ezh2, Eed, and Suz12 (Schwartz, Y. B., et al. (2007) Nat Rev Genet 8, 9-22; Sparmann, A., et al. (2006) Nat Rev Cancer 6, 846-56) and methylates histone H3 at K27 via Ezh2 (Cao, R., et al. (2004) Curr Opin Genet Dev 14, 155-64; 13; Cao, R., et al. (2002) Science 298, 1039-43). Tri-methylated H3K27 facilitates the recruitment of polycomb repressive complex 1 (PRC1), which contains Cbx, Ring, Bmi1, and Mel-18 and promotes gene silencing by ubiquitinating H2A at K119, a histone modification that interferes with the binding of RNA Polymerase II (RNA Pol II; Schwartz, Y. B., et al. (2007) Nat Rev Genet 8, 9-22; and Sparmann, A., et al. (2006) Nat Rev Cancer 6, 846-56; Cao, R., et al. (2002) Science 298, 1039-43; Kirmizis, A., et al. (2004) Genes Dev 18, 1592-605; Zhou, W., et al. (2008) Mol Cell 29, 69-80). RNA Pol II and PRC2 are indeed known to occupy gene promoters in a mutually exclusive manner (Barski, A., et al. (2007) Cell 129, 823-37; Kim, T. H., et al. (2005) Nature 436, 876-80). Replicative senescence in MEFs is characterized by downregulation of Ezh2, elimination of the H3K27 tri-methylation mark at the Ink4a/Arf locus, displacement of Bmi1 and transcriptional activation of Ink4a and Arf (Bracken, A. P., et al. (2007) Genes Dev 21, 525-30; Bracken, A. P., et al. (2006) Genes Dev 20, 1123-36).

Examples herein show that the histone demethylases Ndy1/KDM2B and Ndy2/KMD2A inhibit replicative senescence and immortalize MEFs (Pfau, R., et al. (2008) Proc Natl Acad Sci USA 105, 1907-12 incorporated herein in its entirety; and Examples 1-11 herein). The inhibition of senescence may be caused, at least in part, by the ability of Ndy1 to regulate redox homeostasis and to protect cells from oxidative stress (Polytarchou, C., et al. (2008) Mol Cell Biol 28, 7451-64, hereby incorporated herein by reference in its entirely). Further examples herein show that Ndy1 protects MEFs from replicative senescence, as well as Ras oncogene-induced senescence, by also repressing the Ink4a/Arf locus. Ndy1 mRNA is downregulated in MEFs undergoing senescence. Moreover, overexpression of Ndy1 represses p16^{Ink4a}, and to a lesser extent p19^{Arf} while its knock down has the opposite effect.

Without being limited by any particular theory as to mechanism of action, data herein show that Ndy1 counteracts the senescence-associated downregulation of Ezh2 via a JmjC domain-dependent process and promotes histone H3K27 tri-methylation. The trimethylation of histone H3 in the Ink4a/Arf locus at K27 facilitates the binding of Bmi1. Bmi1 and Ezh2 synergize with Ndy1 to repress the Ink4a/Arf locus suggesting that Ndy1 represses the Ink4a/Arf locus not only by regulating the expression of Ezh2 and the binding if Bmi1 within the locus, but also by additional mechanisms. Data presented in examples herein indeed show that Ndy1 binds the Ink4a/Arf locus and promotes H3K36me2 and H3K4me3 demethylation. These histone modifications combined, interfere with the binding of RNA Pol II and contribute to the silencing of the Ink4a/Arf locus. The effects of Ndy1 on the modification of histones and on the silencing of the Ink4a/Arf locus are passage-dependent, suggesting that Ndy1-induced histone modifications may be amplified by further facilitating the binding of polycomb complexes to this locus.

Ndy1/KDM2B is a JmjC domain-containing histone H3K36me2, H3K36me1 and H3K4me3 demethylase that represses the INK4A/Arf locus and promotes immortalization of mouse embryo fibroblasts. Here we show that Ndy1/KDM2B is induced by FGF-2. Upon induction by FGF-2, and to a lesser degree VEGF but not other growth factors, Ndy1/KDM2B represses the expression of miR-101 by coupling histone H3 K36me2/K36me1/K4me3 demethylation with histone H3 K27 trimethylation in the miR-101 promoter. Repression of miR-101 leads to upregulation of its target Ezh2, which in turn promotes cell proliferation, cell migration, invasiveness and angiogenesis. The FGF-2-Ndy1/KDM2B-miR-101-Ezh2 axis was documented in primary fibroblasts in culture, as well as in set of FGF-2-producing human tumor cell lines. Moreover, analysis of three human lung adenocarcinomas and 40 transitional cell bladder carcinomas, revealed that Ndy1 is frequently overexpressed, relative to normal tissues and that its overexpression correlates well with the repression of miR-101 and the upregulation of Ezh2. These examples define a novel signaling pathway that links FGF-2 signaling to the expression of the histone H3 K27 trimethylase Ezh2, via Ndy1/KDM2B and miR-101 and contributes to tumor progression.

Developing tumors progress through multiple stages. Thus, a carcinoma, composed of transformed epithelial cells, may start in a given organ as a “carcinoma in situ” and progress into an invasive carcinoma and then into a metastatic tumor. The progressive acquisition of increasingly malignant properties depends on sequential genetic and epigenetic changes which reprogram the tumor cells and on the interaction of tumor cells with the surrounding stroma. Tumor cells and stroma are in cell communication and the co-development of these two components of a given tumor is ultimately responsible for the tumor phenotype. Critical components of this co-development are a progressive increase in invasiveness and the “angiogenic switch” of the evolving tumor cells consisting in the acquisition of the ability to induce neovascularization in the surrounding stroma (Carcinogenesis vol. 29 no. 6 pp. 1092-1095, 2008, The Biology of Cancer, June 2006 Garland Science textbooks).

Fibroblast growth factor-2 (FGF-2) or basic fibroblast growth factor (bFGF) is one of 22 members of the FGF growth factor family. FGF-2 is produced by a variety of cell types, binds to heparan sulphate proteoglycans (Blood. 1993 Jun. 15; 81(12):3324-31) in the extracellular matrix and is stored there. Proteolytic degradation of the proteoglycans (J Biol. Chem. 1999 Aug. 27; 274 (35):25167-72, J Vasc Res. 2001 September-October; 38(5):492-501, J Cell Physiol. 1996 March; 166 (3):495-505) leads to FGF-2 release, subsequent binding to the FGF receptors, primarily FGFR-1 and -2, and signaling initiation (Ibrahimi, O. A. et al. (2004) Hum. Mol. Genet. 13, 69-78; Ibrahimi, O. A., et al. (2004) Hum. Mol. Genet. 13, 2313-2324). FGF-2 signaling has been linked to the cycling and differentiation of stem cells during development. Moreover FGF-2 produced either by the tumor cells or the tumor stroma, also plays an important role during oncogenesis. Specifically, it has been shown that FGF-2 stimulates cell proliferation, cell migration invasiveness and angiogenesis and promotes the cycling of cancer stem cells (FEBS Lett. 2006 22; 580(12):2869-74). Molecules targeted by this pathway and contributing to the transduction of FGF-2 signals include MEK1/2, PKC and Akt (Schlessinger, J. (2004) Science 306: 1506-1507). The importance of FGF-2 signaling in oncogenesis was confirmed with use of FGFR inhibitors in animal models of cancer, showing that FGF-2 contributes to proliferation, invasiveness, angiogenesis and metastatic potential of tumor cells (Cancer Research 69, 8645, Nov. 15, 2009. Published Online First Nov. 10, 2009, Clin Cancer Res. 2008 Oct. 1; 14(19):6146-53). Despite progress in understanding the role of FGF-2 in oncogenesis and its role in the “angiogenic switch” in developing tumors however, little is known about the molecular mechanisms that regulate these FGF-2 activities.

Enhancer of zeste homolog-2 (Ezh2) is a K27 histone H3 methyltransferase (Cao, R. et al. (2002) Science 298:1039-1043). Pharmacologic disruption of Polycomb-repressive complex 2-mediated gene repression selectively induces apoptosis in cancer cells (Genes Dev 2007, 21:1050-1063) that is associated with the Polycomb repressor complex-2 (PRC2), one of several Polycomb group complexes (PcGs). The co-recruitment of two of these complexes (PRC1 and PRC2) to Polycomb responsive genes promotes trimethylation of histone H3 at K27 and ubiquitination of histone H2A at K119 and represses gene expression (Gil, J. et al. (2005) DNA Cell Biol 24:117-125; Kamminga, L. M., et al. (2006) Blood 107:2170-2179; Simon, J. A. Nat Rev Mal Cell Biol. 2009 October; 10(10):697-708). In stem cells, PcG complexes counteract the activity of the Trithorax group complexes (TrxG) and the balance between PcG and TrxG complexes regulates the cycling of stem cells and their identity and lineage commitment during differentiation (Cell. 2007 Feb. 23; 128(4):735-45). Ezh2 is a critical component of the machinery that regulates chromatin structure, therefore its overexpression in a variety of cell types may alter their gene expression profiles and several aspects of their biology. Further, overexpression of Ezh2 in cancer cells, a common event to both hematopoietic malignancies and solid tumors, promotes tumor cell proliferation and cell migration and invasiveness, and overexpression in the tumor stroma stimulates angiogenesis (Bracken, A. P., et al. (2003) EMBO J 22:5323-5335; Ding, L. et al., Cancer Res. 2006 Oct. 1; 66(19):9352-5; Berezovska, O. et al. Cell Cycle. 2006 August; 5(16):1886-901; Varambally, S., et al. (2002) Nature 419:624-629). Therefore, there is significant overlap between the phenotypic consequences of FGF-2 and Ezh2 overexpression during oncogenesis.

The upregulation of Ezh2 in different tumors may be transcriptional. More frequently however, Ezh2, which is a target of the microRNA miR-101, is upregulated post-transcriptionally, via the downregulation of this microRNA. The latter may be due to deletion of the miR-101 locus or by unknown mechanisms. Examples herein show that the repression of miR101 is the result of chromatin modifications induced by the histone demethylase Ndy1/KDM2B, which is upregulated by FGF-2 in primary cells, and may also function in tumor cell lines and primary human tumors.

Ndy1/KDM2B is a histone H3K36me2, H3K36me1 and H3K4me3 demethylase that is activated by provirus integration in Moloney murine leukemia virus (MoMuLV)-induced rodent T cell lymphomas. Examples herein show that Ndy1/KDM2B represses the expression of INK4A/Arf, and as a result, it inhibits replicative senescence and promotes immortalization of cultured primary cells. Further examples show that Ndy1/KDM2B is induced by FGF-2 and that the induced protein binds the miR-101 promoter. Promoter binding gives rise to chromatin modifications that repress the expression of miR-101 and result in the upregulation of Ezh2. The FGF-2-Ndy1/KDM2B-miR-101-Ezh2 axis described here was documented in primary fibroblasts in culture, in FGF-2-producing human tumor cell lines and in a set of human transitional cell bladder carcinomas. These examples identify a novel signaling pathway that links FGF-2 to Ezh2 in human cancer and show that the link is epigenetically-regulated via Ndy1/KDM2B, a recently-described histone H3 demethylase.

Embodiments of the invention were published by Raymond Pfau, Alexandros Tzatsos, Sotirios C. Kampranis, Oksana B. Serebrennikova, Susan E. Bear, and Philip N. Tsichlis, in a paper with supplementary data entitled “Members of a family of JmjC domain-containing oncoproteins immortalize embryonic fibroblasts via a JmjC domain-dependent process” which appeared in Proc Natl Acad Sci USA Feb. 12, 2008; 105(6): 1907-1912, and in a paper by Christos Polytarchou, Raymond Pfau, Maria Hatziapostolou, and Philip N. Tsichlis, in a paper entitled “The JmjC domain histone demethylase Ndy1 regulates redox homeostasis and protects cells from oxidative stress”, which appeared in Mol Cell Biol 2008 December; 28(24): 7451-7464, both of which are hereby incorporated herein by reference in their entireties.

### Pharmaceutical Compositions

In one aspect of the present invention, pharmaceutical compositions are provided, such that these compositions include a protein comprising amino acid sequence of at least one Ndy1 gene, and optionally comprise a pharmaceutically acceptable carrier. In certain embodiments, these compositions optionally further comprise one or more additional therapeutic agents. In certain embodiments, the target of choice and/or the additional therapeutic agent or agents are selected from the group of growth factors, anti-inflammatory agents, vasopressor agents, collagenase inhibitors, topical steroids, matrix metalloproteinase inhibitors, ascorbates, angiotensin II, angiotensin III, calreticulin, tetracyclines, fibronectin, collagen, thrombospondin, transforming growth factors (TGF), keratinocyte growth factor (KGF), fibroblast growth factor (FGF), insulin-like growth factors (IGF), epidermal growth factor (EGF), platelet derived growth factor (PDGF), neu differentiation factor (NDF), hepatocyte growth factor (HGF), B vitamins such as biotin, and hyaluronic acid.

As used herein, the term “pharmaceutically acceptable carrier” includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences Ed. by Gennaro, Mack Publishing, Easton, Pa., 1995 describes a variety of different carriers that are used in formulating pharmaceutical compositions and known techniques for the preparation thereof. Some examples of materials that are pharmaceutically acceptable carriers include, but are not limited to, sugars such as glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil, safflower oil, sesame oil, olive oil, corn oil, and soybean oil; glycols; such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

### Therapeutically Effective Dose

In yet another aspect, the methods of treatment of the present invention include the treatment of a cell or cell population in need of immortalization as described herein. Thus, the invention provides methods for the treatment of the cell or cells with an Ndy1 protein or peptide, alone or conjugated for example PEGylated, or a nucleotide sequence encoding such protein or peptide, in such amounts and for such time as is necessary to achieve the desired result. It will be appreciated that this encompasses administering an inventive pharmaceutical as a therapeutic measure to promote the growth and/or inhibit senescence, for example, as a means of producing a tissue for treatment of a condition in need of immortal cells, or to promote differentiation. In certain embodiments of the present invention a “therapeutically effective amount” of the pharmaceutical composition is that amount effective for promoting the condition. The compositions, according to the method of the present invention, may be administered using any amount and any route of administration effective for treating the organ. Thus, the expression “amount effective for promoting the treating the condition”, as used herein, refers to a sufficient amount of composition to promote the immortalization or inhibit senescence. The exact dosage is chosen by the individual physician in view of the cells or the patient to be treated. Dosage and administration are adjusted to provide sufficient levels of the active agent(s) or to maintain the desired effect. Additional factors which may be taken into account include the severity of a disease state, e.g., extent of the condition, history of the condition; age, weight and gender of the patient; diet, time and frequency of administration; drug combinations; reaction sensitivities; and tolerance/response to therapy. Long acting pharmaceutical compositions might be administered several time points a day, every day, 3 to 4 days, every week, or once every two weeks depending on half-life and clearance rate of the particular composition.

The active agents of the invention are preferably formulated in dosage unit form for ease of administration and uniformity of dosage. The expression “dosage unit form” as used herein refers to a physically discrete unit of active agent appropriate for the cells or patient to be treated. It will be understood, however, that the total daily usage of the compositions of the present invention are decided by an attending physician, within the scope of sound medical judgment. For any active agent, the therapeutically effective dose can be estimated initially either in cell culture assays or in animal models, usually mice, rabbits, dogs, or pigs. The animal model is also used to achieve a desirable concentration range and route of administration. While direct application to the cell is envisioned as the route of administration, in vivo or ex vivo, such information can then be used to determine useful doses and additional routes for administration in animals or humans.

A therapeutically effective dose refers to that amount of active agent that ameliorates the symptoms or condition. Therapeutic efficacy and toxicity of active agents can be determined by standard pharmaceutical procedures in cell cultures or experimental animals, e.g., ED50 (the dose is therapeutically effective in 50% of the population) and LD50 (the dose is lethal to 50% of the population). The dose ratio of toxic to therapeutic effects is the therapeutic index, and it can be expressed as the ratio, LD50/ED50. Pharmaceutical compositions that exhibit large therapeutic indices are preferred. The data obtained from cell culture assays and animal studies is used in formulating a range of dosage for human use.

### Administration of Pharmaceutical Compositions

After formulation with an appropriate pharmaceutically acceptable carrier in a desired dosage, the pharmaceutical compositions of this invention can be administered to humans and other mammals topically such as transdermally (as by powders, ointments, or drops), i.e., as applied directly to the skin or mucosa. Alternative and additional routes such as orally, rectally, parenterally, intracisternally, intravaginally, intraperitoneally, bucally, or nasally, depending on the severity of the condition being treated, are envisioned.

Liquid dosage forms for administration include buffers and solubilizing agents, preferred diluents such as water, preservatives such as thymosol, and one or more biopolymers or polymers for conditioning the solution, such as polyethylene glycol, hydroxypropylmethylcellulose, sodium hyaluronate, sodium polyacrylate or tamarind gum.

Liquid dosage forms for oral administration include, but are not limited to, pharmaceutically acceptable emulsions, microemulsions, solutions, suspensions, syrups and elixirs. In addition to the active agent(s), the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Dosage forms for topical or transdermal administration of an inventive pharmaceutical composition include ointments, pastes, creams, lotions, gels, powders, solutions, sprays, inhalants, or patches. The active agent is admixed under sterile conditions with a pharmaceutically acceptable carrier and any needed preservatives or buffers as may be required. For example, ocular administrations are aqueous drops, a mist, an emulsion, or a cream. Administration may be therapeutic or it may be prophylactic. Prophylactic formulations may be present or applied to the site of potential wounds, or to sources of wounds, such as contact lenses, contact lens cleaning and rinsing solutions, containers for contact lens storage or transport, devices for contact lens handling, eye drops, surgical irrigation solutions, ear drops, eye patches, and cosmetics for the eye area, including creams, lotions, mascara, eyeliner, and eyeshadow. The invention includes devices, surgical devices, audiological devices or products which contain disclosed compositions (e.g., gauze bandages or strips), and methods of making or using such devices or products. These devices may be coated with, impregnated with, bonded to or otherwise treated with a disclosed composition.

The ointments, pastes, creams, and gels may contain, in addition to an active agent of this invention, excipients such as animal and vegetable fats, oils, waxes, paraffins, starch, tragacanth, cellulose derivatives, polyethylene glycols, silicones, bentonites, silicic acid, talc, zinc oxide, or mixtures thereof.

Powders and sprays can contain, in addition to the agents of this invention, excipients such as talc, silicic acid, aluminum hydroxide, calcium silicates, polyamide powder, or mixtures of these substances. Sprays can additionally contain customary propellants such as chlorofluorohydrocarbons.

Transdermal patches have the added advantage of providing controlled delivery of the active ingredients to the body. Such dosage forms are made by dissolving or dispensing the compound in the proper medium. Absorption enhancers can also be used to increase the flux of the compound across the skin. The rate is controlled by either providing a rate controlling membrane or by dispersing the compound in a polymer matrix or gel.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use. In order to prolong the effect of an active agent, it is often desirable to slow the absorption of the agent from subcutaneous or intramuscular injection. Delayed absorption of a parenterally administered active agent is accomplished by dissolving or suspending the agent in an oil vehicle. Injectable depot forms are made by forming microencapsule matrices of the agent in biodegradable polymers such as polylactide-polyglycolide. Depending upon the ratio of active agent to polymer and the nature of the particular polymer employed, the rate of active agent release can be controlled. Examples of other biodegradable polymers include poly(orthoesters) and poly(anhydrides). Depot injectable formulations are also prepared by entrapping the agent in liposomes or microemulsions which are compatible with body tissues.

Compositions for rectal or vaginal administration are preferably suppositories which can be prepared by mixing the active agent(s) of this invention with suitable non-irritating excipients or carriers such as cocoa butter, polyethylene glycol or a suppository wax which are solid at ambient temperature but liquid at body temperature and therefore melt in the rectum or vaginal cavity and release the active agent(s).

Solid dosage forms for oral administration include capsules, tablets, pills, powders, and granules. In such solid dosage forms, the active agent is mixed with at least one inert, pharmaceutically acceptable excipient or carrier such as sodium citrate or dicalcium phosphate and/or a) fillers or extenders such as starches, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof.

Solid compositions of a similar type may also be employed as fillers in soft and hard-filled gelatin capsules using such excipients as milk sugar as well as high molecular weight polyethylene glycols and the like. The solid dosage forms of tablets, dragees, capsules, pills, and granules can be prepared with coatings and shells such as enteric coatings, release controlling coatings and other coatings well known in the pharmaceutical formulating art. In such solid dosage forms the active agent(s) may be admixed with at least one inert diluent such as sucrose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active agent(s) only, or preferentially, in a certain part of the intestinal tract, optionally, in a delayed manner. Examples of embedding compositions which can be used include polymeric substances and waxes.

An embodiment of the invention herein provides a pharmaceutical composition for immortalizing and reversing senescence of a cell which includes an effective dose of at least one of a mammalian Ndy protein-related composition selected from the group of: an Ndy1 protein; an Ndy2 protein; a vector encoding an Ndy1 nucleotide sequence; a vector encoding an Ndy2 nucleotide sequence; a modulator of Ndy1 expression; a modulator of Ndy2 expression; wherein the protein, vector and modulator function to increase cellular amount or activity of functional long form Ndy protein, the long form protein having a functional JmjC domain having histone demethylase activity.

An alternative embodiment of the invention herein provides a pharmaceutical composition for inhibiting immortalization and stimulating differentiation of a cell including an effective dose of at least one mammalian short form Ndy protein-related composition selected from the group of: a short form Ndy1 protein; a short form Ndy2 protein; a vector encoding a short form Ndy1 nucleotide sequence; a vector encoding a short form in Ndy2 nucleotide sequence; a modulator of short form Ndy1 expression; a modulator of short form Ndy2 expression; wherein the short form Ndy protein, vector and modulator function to increase cellular amount or activity of short form Ndy protein lacking a functional JmjC domain thereby lacking demethylase activity, wherein the short form Ndy protein further inhibits histone demethylase Ndy long form expression or activity.

Related embodiments of the pharmaceutical compositions according to either of the above further include a pharmaceutical buffer. Related embodiments of the pharmaceutical compositions for regulating immortalization and senescence in a cell further include an effective ratio of a each of a long form and a short form Ndy protein-related compositions, such that the relative amount in the composition of the long form compared to the short form Ndy protein is adjusted to regulate amount of histone demethylation and gene expression to direct cell expression, wherein a greater ratio of long form to short form promotes immortalization, and a lower ratio promotes differentiation and/or senescence. For example, in the pharmaceutical composition, immortalization is promoted by a ratio of greater than about 1:1, or at least about 2:1, 5:1, or 10:1 of long form to short form. For example, in the pharmaceutical composition, differentiation and/or senescence is promoted by a ratio of less than about 1:1, or at least about 1:2, 1:5, or 1:10 of long form to short form. The pharmaceutical composition according to any of above further includes a pharmaceutically acceptable buffer or salt.

Another embodiment provides a method of immortalizing a cell comprising contacting the cell with a composition selected from the group of:

a vector carrying a nucleotide sequence of an Ndy1 gene operably linked to regulatory signals to promote expression of the Ndy1 gene, such that the Ndy1 gene includes information encoding a functional JmjC domain;

a vector carrying a nucleotide sequence of an Ndy2 gene operably linked to regulatory signals to promote expression of the Ndy2 gene, such that the Ndy2 gene includes information encoding a functional JmjC domain; and,

a vector carrying a nucleotide sequence that negatively modulates expression of a short form Ndy1 and/or Ndy2 gene. In a related method, the cell is cultured ex vivo and is immortalized for maintaining of a stem-cell like phenotypes during growth, amplification and subsequent passaging and storage. For example, the cell is an embryonic stem cell or a hemopoietic stem cell. For an alternative example, the immortalized cell is in vivo in a subject suffering from a senescence condition. In the related method, the ex vivo cell is further implanted in vivo. For example, the senescence condition is neurological, muscular, hematopoietic, or dermatological. Alternatively, the condition is selected from Alzheimers, pre-Alzheimers, amnesia, psychosis, muscular dystrophy, myotonic dystrophy, sickle cell anemia, thallasemia, and progeria.

An alternative embodiment provides a method of promoting differentiation senescence a cell comprising contacting the cell with a composition selected from the group of:

a vector carrying a nucleotide sequence of short form of an Ndy1 gene operably linked to regulatory signals to promote expression of the Ndy1 gene, wherein the Ndy1 gene lacks information encoding a functional JmjC domain;

a vector carrying a nucleotide sequence of short form of an Ndy2 gene operably linked to regulatory signals to promote expression of the Ndy2 gene, wherein the Ndy2 gene lacks information encoding a functional JmjC domain; and,

a vector carrying a nucleotide sequence that upregulates expression of a short form Ndy1 and/or Ndy2 gene. In a related example, the cell is in vivo in a subject suffering from a cancer or a neoplastic condition. For example, the cell is cultured ex vivo and is further implanted in vivo. In related embodiments, the cancer is hematopoietic. For example, the hematopoietic cancer is a leukemia or a lymphoma. Alternatively, the cancer is selected from the group of prostate, testicular cancer, breast, colon, ovarian, bladder, transitional cell carcinoma, pancreatic, esophageal, lung, brain, melanoma, and basal cell carcinoma.

In a related embodiment, the cancer is a sarcoma and the method further comprises treating with an additional anti-tumor agent. For example, the additional anti-tumor agent is selected from the group of radiation, thermal disruption, and angiogenesis inhibition.

An embodiment of the invention provides a method of obtaining an anti-Ndy antibody comprising contacting an animal with a peptide 907-KMRRKRRLVNKELSKC-921 (SEQ ID NO:2) or a fragment thereof. For example, the fragment is at least four amino acids to seven amino acids in length. In general, the antibody recognizes and binds to an Ndy protein from a mammal.

An embodiment of the invention provides a method of prognosing or diagnosing a cell or tissue for susceptibility to a cancer, the method comprising:

contacting a sample of the cell or tissue with an antibody or a nucleotide sequence, respectively, that specifically binds, respectively, to an Ndy antigen or a nucleotide sequence of an Ndy gene;

observing an amount of binding of the antibody or the nucleic acid; and,

analyzing the amount in comparison to that to a negative control normal cell or tissue, wherein an increase in an Ndy antigen in comparison to the negative control provides a prognosis or a diagnosis of the cell or tissue. In a related embodiment, the method further includes determining extent in the amount of a ratio of Ndy long form compared to Ndy short form, wherein an increase in long form compared to short form is a prognosis or diagnosis of susceptibility to cancer.

An embodiment of the invention provides a method of identifying a compound capable of binding to and inhibiting activity of an Ndy protein, the method including the steps of:

contacting the compound to a first cell having an Ndy retroviral construct, wherein the Ndy construct encodes a long form having a JmjC domain; and,

observing differentiation morphology of a contacted first cell, wherein the compound is identified as promoting differentiation of the first cell, in comparison with that of a second cell identically having the Ndy retroviral construct and is a control not so contacted with the compound, and further in comparison with a third cell lacking the retroviral construct and is a control similarly contacted with the compound, wherein the compound results differentiation of the contacted first cell in comparison to the second and third cells. For example, each of the first, second and third cell or tissue is in culture. In a related embodiment, each of the cell or tissue is a plurality of cells, cell populations, or tissue cultures, wherein each of the plurality is located in a well of a multi-well culture dish. In another related embodiment the compound is one of a plurality of compounds. In a related embodiment, observing further comprises measuring a marker of differentiation that is at least one parameter which is immunologic, colorimetric, fluorimetric, fluorescent, radioactive, or enzymatic.

Also provided herein is a method of treating a subject having a cancer such as a breast cancer, a testicular cancer, a leukemia or lymphoma, the method comprising contacting the subject with a vector carrying an siRNA that inhibits expression of an Ndy protein, wherein the Ndy protein comprises a JmjC domain and the siRNA inhibits expression of endogenous Ndy protein and function or activity of the JmjC domain.

An embodiment of the invention herein provides a method of identifying a compound capable of binding to and inhibiting activity of an Ndy protein, the method comprising:

contacting the compound to a first sample of an Ndy protein, wherein the Ndy protein comprises a JmjC domain in an in vitro assay comprising at least one methylated substrate, and under conditions suitable for histone demethylation; and,

observing inhibition by the compound of an amount of an enzymatic reaction of a demethylase product of the substrate, wherein the compound is identified as inhibiting the amount produced in first sample, in comparison with that of a second control sample identically having the Ndy protein and not so contacted with the compound, wherein the compound results decrease of the demethylation product in the first sample in comparison to the second sample.

In a related embodiment, observing further includes a third sample which is a control having substrate and lacking the Ndy protein, wherein observing the third sample is measuring spontaneous non-enzymatic background demethylation of the substrate. In various embodiments, the Ndy is a long form of Ndy1 or Ndy2. Further, the Ndy protein is selected from the group of: a crude cells extract; an enriched fraction prepared from a mouse cell extract by preparative immunoprecipitation; and a bacterially produced isolated protein. In a related embodiment, contacting the protein with a composition further includes simultaneously contacting the protein with a plurality of identified compounds in a sibling pool. In a related embodiment, contacting the protein with a composition further includes simultaneously contacting a plurality of protein samples with a plurality of identified compounds in a high throughput multi-well format. In a related embodiment, the methylated substrate is bulk histone and observing includes a Western blot of an SDS-PAGE. In an alternative embodiment, the methylated substrate is a di-methylated or a tri-methylated isolated synthetic peptide and observing includes measuring a change in fluorescence. For example, the di-methylated or tri-methylated isolated synthetic peptide is at least one of ART-K(me₃)-QTARKST and ATGGV-K(me₂)-KPHRY. Further, measuring a change in fluorescence is in an exemplary embodiment, monitoring oxidation of product formaldehyde by a glutathione-independent formaldehyde dehydrogenase which reduces NAD⁺ to NADH. In general, suitable conditions include presence of α-ketoglutarate and an iron salt. In a related embodiment, the method further includes observing anti-cancer activity of the compound.

Also provided herein is use of a composition that includes an Ndy amino acid sequence or a nucleotide sequence encoding an Ndy amino acid sequence to treat a cancer or a senescence condition of a cell, such that the use comprises formulating a medicament comprising the composition in a pharmaceutically acceptable buffer or salt, and contacting the cell with the composition. A related embodiment further includes formulating the composition in an effective dose.

Another embodiment provided herein is a method for inhibiting growth of cells, the method involving contacting the cells with an siRNA capable of inhibiting Ndy1 expression. For example, the siRNA comprises a ribonucleic acid sequence

| | |
|---|---|
| 1433-GUGGACUCACCUUACCGAAUU-1454. | (SEQ ID NO: 1) |

Additional embodiments and examples of the invention are found in the claims below, which are for illustrative purposes and are not to be construed as further limiting.

### EXAMPLES

### Example 1

### Cloning of Ndy1 and Ndy2 from MoMuLV-Induced Rat T-Cell Lymphomas

Newborn Fisher-344 rats were injected with 10⁵ plaque-forming units (PFUs) of MoMuLV intraperitoneally, monitored for tumor development, and sacrificed before death. Provirus integration sites were cloned from tumor cell DNA by inverse PCR as previously described (Gilks, C. B. et al. (1993) Mol Cell Biol 13, 1759-68).

Provirus integration in the Ndy1 and Ndy2 loci was confirmed by Southern blotting. The sequence of the cell DNA-derived portion of the clones was blasted against the fully-sequenced rat genome. Rat, mouse and human cDNA clones of Ndy1 and a mouse clone of Ndy2 were generated by RT-PCR, using oligonucleotide primers designed on the basis of the results of the blast analysis.

To clone the rat, mouse and human Ndy1 and the mouse Ndy2 cDNAs, RT-PCR was employed, using the following oligonucleotide primers: (Rat Ndy1): 5′GGGCAGGAGTGTTGACAATTA-′3 (SEQ ID NO: 3) sense and 5′-CATCCTTGTCCTGGAGCTAA-′3 antisense (SEQ ID NO: 4).

(Mouse Ndy1): 5′-GACTTTGCAAACGGATCTGC-′3 (SEQ ID NO: 5) sense and 5′CGCCTTAGAAATGGTCCAGA-′3 antisense (SEQ ID NO: 6).
(Human Ndy1): 5′CTGCCTAAGTGTTGGTGCAA-′3 sense (SEQ ID NO: 7) and 5′-CAGGAAAATTGGCAATCTCAA-′3 antisense (SEQ ID NO: 8).
(Mouse Ndy2): 5′-CATCCCTGGAGTGGTTTCTT-′3 sense (SEQ ID NO: 9) and 5′GCAGAGAGGGAATGTGTCGT-′3 antisense (SEQ ID NO: 10).

### Example 2

### Plasmid Constructs

Retroviral constructs were based on the retrovirus vector MigR1, a variant of MigR1 in which the green fluorescent protein (GFP) gene was replaced by the red fluorescent protein (RFP) gene and pBabe-puro. Ndy1, Ndy2 and p21^{CIP1} were cloned within the multiple cloning sites of these vectors and tagged at the carboxy-terminus with either myc or HA tags. Retroviral constructs of Ndy1 deletion mutants were generated from the wild type construct by overlap extension PCR. Retroviral constructs of Ndy1 deletion mutants were generated from the wild type constructs by overlap extension PCR as follows. The DNA sequences on either side of the planned deletion were amplified using two sets of oligonucleotide primer pairs. The oligonucleotide flanking the 5′ end of the deletion, contained a 15 nucleotide tail that was complementary to the sequences flanking the 3′ end of the deletion, and the oligonucleotide at the 3′ end of the deletion contained a tail complementary to the sequences 5′ of the deletion. The amplified pieces were mixed, and the assembled full length cDNA, minus the deleted sequences was amplified by PCR, using the primers at the 5′ and the 3′ end of the full length cDNA clone. The following primers were used: (i) Ndy1 ΔJmjC (Ndy1 Δ198-298) 5′-CCCAAAGTGAAAAAGCTGCATAGCTTCAAC-′3 sense (SEQ ID NO: 11) and 5′-GTTGAAGCTATGCAGCTTTTTCACTTTGGG-′3 antisense (SEQ ID NO: 12),

(ii) Ndy1 ΔCxxC (Ndy1 Δ579-625) 5′-CGAACAACAGCAGGAGCGCCAGTGCTGCCC-3′ sense (SEQ ID NO: 13) and 5′-GGGCAGCACTGGCGCTCCTGCTGTTGTTCG-′3 antisense (SEQ ID NO: 14),
(iii) Ndy1 ΔPHD (Ndy1 Δ633-697) 5′-GTGCTGCCCCACACCGGCAAGACCGGGAAA-′3 sense (SEQ ID NO: 15) and 5′-TTTCCCGGTCTTGCCGGTGTGGGGCAGCAC-′3 antisense (SEQ ID NO: 16),
(iv) Ndy1 ΔPRR (Ndy1 Δ987-1029) 5′-CGGCACTCGCTGGGACTGGATGATGGAGCA-′3 sense (SEQ ID NO: 17) and 5′-TGCTCCATCATCCAGTCCCAGCGAGTGCCG-′3 antisense (SEQ ID NO: 18),
(v) Ndy1 ΔF-box (Ndy1 Δ1033-1077) 5′-CTGCCCCTGGATGATGACCTGAACCGCTGC-′3 sense (SEQ ID NO: 19) and 5′-GCAGCGGTTCAGGTCATCATCCAGGGGCAG-′3 antisense (SEQ ID NO: 20), and
(vi) Ndy1 ΔLRR (Ndy1 Δ1104-1309) 5′-CTAGTCAAGGGAGACAGGCTGTCG-′3 antisense (SEQ ID NO: 21).

The point mutants Ndy1 Y221A, Ndy1 H283Y and Ndy2H212A (mouse) were generated from the wild type mouse cDNA, using the Quikchange XL mutagenesis kit (Stratagene #200517; Garden Grove, Calif.) and the following primers: (Ndy1 Y221A mutant): 5′GGAGGCACCTCCGTGTGGGCCCATGTGTTCCGTGGTGG-′3 (SEQ ID NO: 22),

(Ndy1 H283Y mutant): 5′-CCCTTCAGGTTGGATCTATGCGGTTTATACGCCTG (SEQ ID NO: 23), and (Ndy2H212 mutant): 5′-CGAGGCTGCTATACTGACTTCGCTGTGGATTTTGGAGGTACTTC-3′ (SEQ ID NO: 24).

Retroviruses expressing the wild-type and mutant forms of Ndy1 and Ndy2 were packaged in HEK293T cells. Infections of MEFs were carried out in the presence of polybrene (8 μg/ml). Forty eight hours post-infection GFP-positive cells were sorted by FACS (MoFlo, Dako-Cytomation, University of California, Berkeley, Calif.). Cells were counted and plated in triplicate in 12-well plates (5×10⁴ cells/well). Cells were grown for 3.5 days, and then harvested, counted and replated also at 5×10⁴ cells/well. This cycle was repeated multiple times with parallel monitoring of the growth rate and the morphology of the cells, as well as the expression of senescence-associated β-galactosidase.

In later examples, retrovirus constructs were packaged in 293 T cells transiently transfected with the respective constructs and with an ecotropic virus Env construct (for infection of murine cells) or an amphotropic virus Env construct, using Fugene 6 (11814443001, Roche Applied Science). Cells were infected with the packaged viruses as follows: Cells were pre-treated with DEAE dextran (25 μg/ml). Forty five minutes later, they were washed and infected. Infected cells were sorted for GFP or selected with puromycin (for HA.Ndy1, myc.Ezh2, 2 μg/ml) or bleomycin (for myc.Ezh2, 100 μg/ml)) 48 hours later. If multiple infections were to be performed, cells were infected and selected before the next infection cycle begun.

Lentiviral-based shRNAs for human Ndy1 were purchased from Open Biosystems (catalog number RH54533-NM_{—}001005366). Lentivirus constructs were packaged in 293 T cells transiently transfected with these constructs and with pCMV-VSVG and pCMV-dR8.2 dvpr. Out of the 5 clones, the most effective one in knocking down human Ndy1 was clone TRCN0000118437. Knockdown efficiency was tested with western blot.

RNAs were transfected with Lipofectamine 2000 (Invitrogen, catalog number 11668-027) in MEFs and DharmaFect 3 (Dharmacon, catalog number T-2003-03) in cancer cell lines, according to the manufacturers' instructions (80 nM final concentration).

For stimulation with FGF-2 (Cell signalling, catalog number 8910), IGF-1 (R&D Systems, catalog number 791-MG), PDGF (Cell signalling, catalog number 9909), VEGF (R&D systems, catalog number 493-MV), EGF (R&D systems, catalog number 236-EG), TNFα (Cell Signalling, catalog number 2169), cells were serum starved for 16 hours and growth factors were added at a final concentration of 20 ng/ml each, for 24 hours.

TROLOX ((±)-6-Hydroxy-2,5,7,8-tetramethylchromane-2-carboxylic acid, (Sigma, catalog number 238813), NAC (N-Acetyl-L-cysteine, Sigma, catalog number A9165), H₂O₂ (Sigma, catalog number H-1009) and PD173074 (N-[2-[[4-(Diethylamino)butyl]amino]-6-(3,5-dimethoxyphenyl)pyrido[2,3-d]pyrimidin-7-yl]-N′-(1,1-dimethylethyl) urea, FGFR-1/3 inhibitor, TOCRIS Bioscience, catalog number 3044) were added directly in the culture at the indicated concentrations.

### Example 3

### Cell Culture, siRNA and Senescence Assays

IMR90 (CCL186) and HEK293T (CRL-11268) cells were obtained from ATCC. MEFs were isolated from 13.5 day C57B1/6 mouse embryos. MEFs, IMR90, and HEK293T cells were cultured in Dulbecco's modified Eagle's minimal essential medium supplemented with 10% fetal bovine serum (FBS), penicillin and streptomycin, and nonessential amino acids. The human colon cancer cell lines HCT-116(p53^{-/-}) and HCT116(p53^{+/+}) (Bunz, F. et al. (1998) Science 282, 1497-501) were grown in McCoy's 5a medium supplemented with 10% FBS. To generate HCT-116 cells stably overexpressing Ndy1, the parental HCT-116 cells were infected with pBabeNdy1.Myc or pBabe-puro and the infected cells were selected for puromycin resistance (2 μg/ml) for two days. Infections of MEFs were carried out in the presence of polybrene (8 μg/ml).

MEFs overexpressing Ndy1 or Ndy2 and MEFs in which Ndy1 was knocked down with siRNA (1433-GUGGACUCACCUUACCGAAUU-1454, SEQ ID NO: 1), were monitored for senescence by light microscopy and β-galactosidase staining and by cell counting at each passage. Transfection of siRNA was carried out using Lipofectamine 2000 (Invitrogen; Carlsbad, Calif.). Knockdown of Ndy1 was confirmed by Western blotting and real time RTPCR.

Cells infected with the empty MigR1 vector, or MigR1-based Ndy1 or Ndy2 retroviral constructs were counted and plated in triplicate in 12-well plates (5×10⁴ cells/well). Cells were grown for 3.5 days, and then harvested, counted and replated also at 5×10⁴ cells/well. This cycle was repeated multiple times with parallel monitoring of the growth rate and the morphology of the cells, as well as the expression of senescence-associated β-galactosidase.

MEFs transfected with Ndy1 (1433-GUGGACUCACCUUACCGAAUU-1454) or control siRNA (Dharmacon; Chicago, Ill.) were plated in triplicate at 10⁵ cells per well in 6 well dishes, and three days later they were counted, re-transfected and replated at 10⁵ cells per well. At day 6, cells were photographed, counted and stained for the β-galactosidase (Pierce #9860; Rockford, Ill.). β-galactosidase staining of the cells infected with the retroviral constructs and the siRNA-treated cells was performed using X-gal and a β-galactosidase staining kit (Pierce #9860).

### Example 4

### Gene Expression Analysis: Northern Blotting, Real Time RT-PCR, Antibodies and Western Blotting

Northern blotting and real time RT-PCR were carried out using standard procedures as described herein. To measure the expression of the Ndy1 protein in both tumors and MEFs, Western blots of nuclear cell lysates were probed with anti-Ndy1, anti-Myc or anti-HA antibodies (Suppl. data). The polyclonal antibody specific for Ndy1 was obtained by injecting rabbits with the peptide 907-KMRRKRRLVNKELSKC-921, which maps between the PHD2 and F-Box domains. The position of the N-terminal and C-terminal amino acids was based on the sequence of the mouse protein. The antibody was observed to recognize the mouse, human and rat Ndy-1.

RNA was isolated from MoMuLV induced tumors using TRIZOL reagent (Invitrogen #15596-026). A sample having 15 μg total RNA was resolved in a 1% agarose-formaldehyde gel and probed with a full length rat Ndy1 cDNA probe. To measure the expression of Ndy1 and Ndy2 by real time PCR RNA was isolated using the RNeasy mini kit (Qiagen #74104; Valencia, Calif.). cDNAs of these RNAs were synthesized with RETROscript (Ambion #AM1710; Austin, Tex.). To measure the expression of Ndy1, PCR reactions were carried out in triplicate in a final volume of 25 μl containing the template cDNA, iQ SYBRGreen Supermix (Bio-Rad #170-8882; Irvine, Calif.) and the following primers:

(mouse Ndy1): 5′AGACACCAGAGGCACAGAGG-′3 sense (SEQ ID NO: 25) and 5′-CACAGTGGGACGCTTGACTA-′3 antisense (SEQ ID NO: 26). (GAPDH control): 5′-TGTGTCCGTCGTGGATCTGA-′3 sense (SEQ ID NO: 27) and 5′CCTGCTTCACCACCTTCTTGA-′3 antisense (SEQ ID NO: 28). Data were analyzed using an Opticon2 continuous fluorescence detector (MJ Research). Cycling conditions were as follows: 95° C. for 10 min, followed by 40 amplification cycles (95° C. for 15 sec, 55° C. for 35 sec and 72° C. for 30 sec). To measure the expression of Ndy2, the following primers were used:

5′ CAAGCAGGG CTATACCTTCG 3′
(Forward primer (SEQ ID NO: 29)
and
5′ GGGGATGTTAAAGCTATG CAA 3′.
(Reverse primer (SEQ ID NO: 30)

Total RNA was isolated with Trizol (Invitrogen, catalog number 15596-026), according to the manufacturer's instructions. Real-time PCR analysis was performed to determine the expression levels of miR-101 in cultured cells and in tumors. The expression levels were normalized to U6 small nuclear RNA (internal control). Reverse transcription was performed using First-strand cDNA synthesis kit (Exiqon, catalog number 201100) with specific primer sets for miR-101 (Exiqon, catalog number 202133) and U6 snRNA (Exiqon, catalog number 201510). Real-time PCR was performed in duplicate using the SYBR Green master mix (Exiqon, catalog number 201000) on an Opticon² DNA Monitor instrument (Biorad).

Complementary DNA (cDNA) was synthesized from 1.0 μg of total RNA by oligo-dT priming, using the Retroscript reverse transcription kit (Ambion, catalog number AM1710). Real-time PCR was performed in triplicate using the RT² Real-Time SYBR Green PCR master mix system (SuperArray Bioscience Corporation, catalog number PA-110) on an Opticon² DNA Monitor instrument (Biorad). mRNA quantification data were normalized to GAPDH, as an internal control.

Chromatin immunoprecipitation (ChIP) was performed using the Chromatin Immunoprecipitation assay kit (Millipore, catalog number 17-295) according to the manufacturer's instructions. Briefly, proteins were cross-linked to DNA by formaldehyde. Cells were lysed and sonicated to shear DNA to 300-bp to 500-bp fragments. A fraction of each sample was then precleared with protein A- and salmon sperm DNA-bound agarose beads. Following overnight incubation with the immunoprecipitating antibody and 1 h of incubation with protein A- and agarose-salmon sperm DNA beads at 4° C., the immunoprecipitates were subjected to multiple washes. DNA recovered after reversion of the protein-DNA cross-links with NaCl was incubated with proteinase K. Subsequently, it was extracted with phenol-chloroform and precipitated with ethanol. Real-time PCR using different sets of primers to amplify the miR-101 and prdx2 genomic loci was employed in both the input and the immunoprecipitated DNA.

Western blots were performed using a nuclear/cytoplasmic isolation kit (Pierce #78833). To determine whether Ndy1 was chromatin-bound, cells were washed twice in ice-cold PBS and solubilized in the lysis buffer (50 mM Tris, pH 7.5, 200 mM NaCl, 1% Triton X-100, 0.1% SDS, 10 mM Na₃VO₄, 50 mM NaF, 1 mM β-glycerophosphate, 1 mM sodium pyrophosphate, 1 mM EDTA, 1 mM EGTA, 1 mM PMSF, supplemented with a mixture of protease inhibitors). The lysates were sonicated in a Misonix 3000 sonicator for 5 seconds at power level 2 and they were centrifuged for 20 min at 16,000×g. The supernatant (soluble whole cell lysate), and the insoluble pellet, which is highly enriched in histones and other chromatin-associated proteins, were analyzed by Western blotting.

### Example 5

### Immunostaining

Low passage MEFs were infected with the empty MigR1 vector or a MigR1Ndy1.HA construct. Cells were fixed by exposure to 4% paraformaldehyde for 10 min and then they were washed and permeabilized with 0.2% Triton-X100. Permeabilized cells were incubated for 1 hr at room temperature with a mouse monoclonal anti-HA antibody (Covance; Madison, Wis.). Following washing of the primary antibody, the cells were incubated with PE-conjugated anti-mouse IgG secondary antibody.

### Example 6

### Ndy1 is a Common Target of Provirus Integration in MoMuLV-Induced Rat T Cell Lymphomas

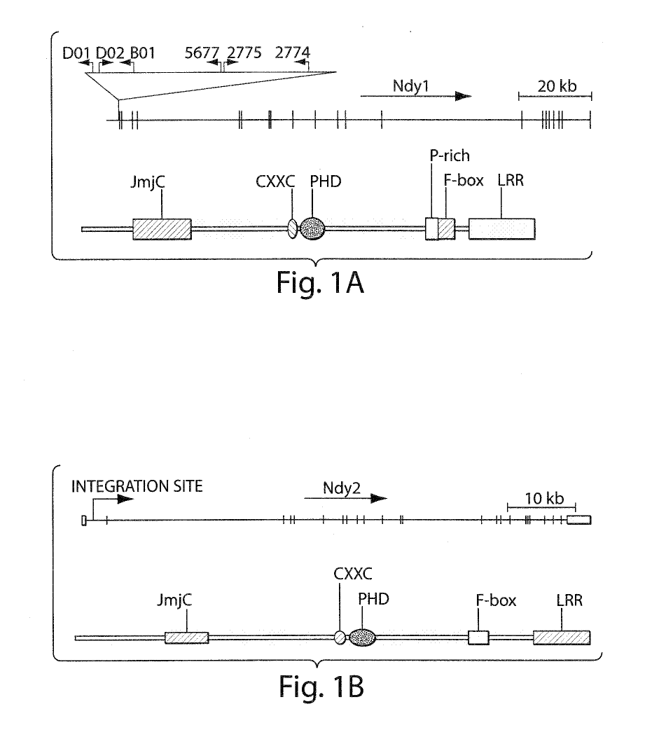

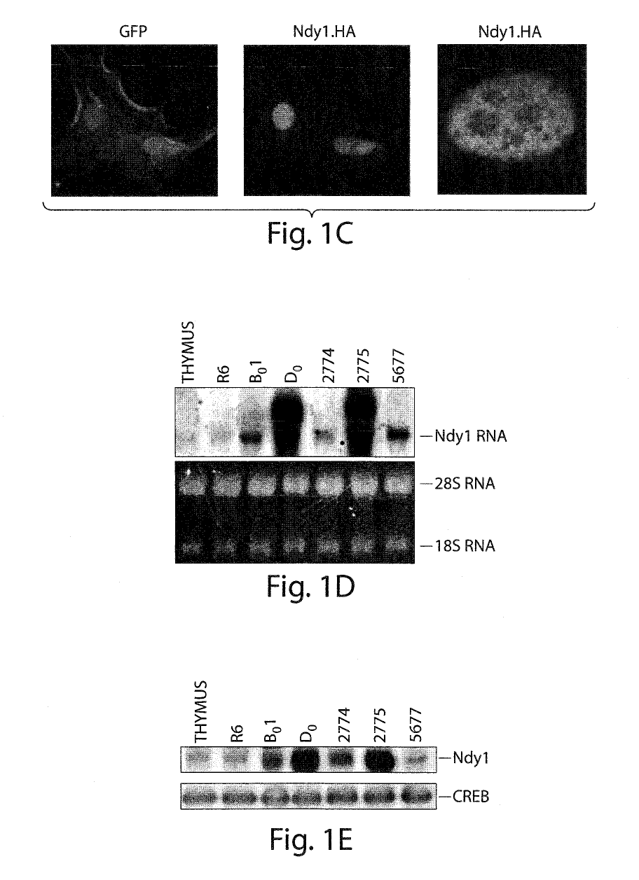

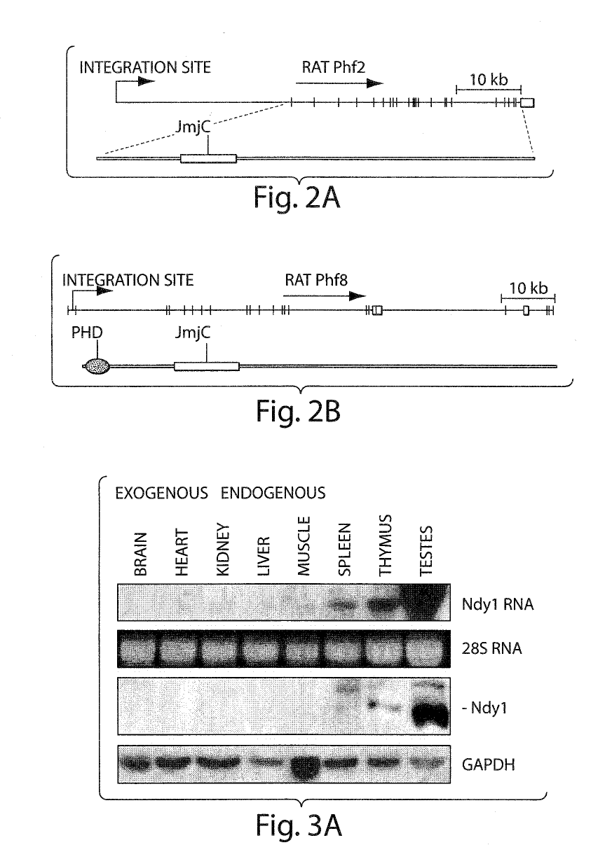

A genome-wide screen of 44 MoMuLV induced T-cell lymphomas for novel targets of proviral DNA integration, yielded clones of 149 independent provirus integrations. Six of these integrations, cloned from five independent tumors, had targeted a gene named Ndy1, also known as FBXL10 or JHDM1B (Tsukada, Y. et al. (2006) Nature 439, 811-6; Klose, R. J. et al. (2006) Nat Rev Genet 7, 715-27; Suzuki, T. et al. (2006) Embo J 25, 3422-31; Frescas, D. et al. (2007) Nature 450, 309-13). Two were cloned from a single tumor, D0, indicating that this tumor consists of at least two populations of cells, both of which carry an integrated provirus in this locus. The integrations in the vicinity of Ndy1 detected to-date were located within a region 100 by upstream of one of several transcription initiation sites utilized by this gene. The transcriptional orientation of two of the five integrated proviruses was the same as that of the Ndy1 gene (FIG. 1 panel A). A single provirus insertion was also detected immediately upstream of the Ndy1 homolog Ndy2, also known as FBXL11 or JHDM1A (FIG. 1B). Interestingly, provirus insertions were also detected upstream of two more genes encoding JmjC domain-containing proteins, Phf2 and Phf8 (FIG. 2). The Ndy1 and Ndy2 homologs were chosen for detailed characterization.

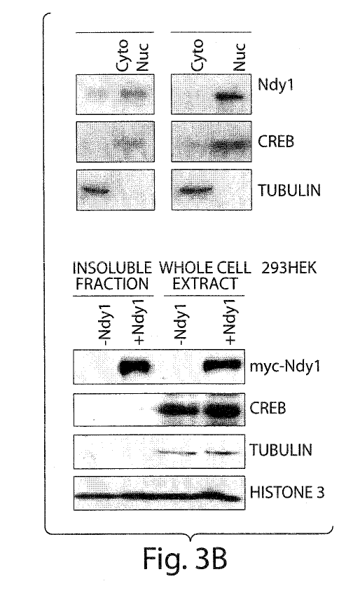

Ndy1 encodes a protein which contains a JmjC domain (Tsukada, Y. et al. (2006), Nature 439, 811-6; Klose, R. J. et al. (2006) Nat Rev Genet 7, 715-27; Frescas, D. et al. (2007) Nature 450, 309-13; Suzuki, T. et al. (2006) Embo J 25, 3422-31), a CXXC zinc finger, a PHD zinc finger, a proline-rich region, an F-box, and a leucine-rich repeat (LRR; FIG. 1 panel A; Klose, R. J. et al. (2006) Nat Rev Genet 7, 715-27). Ndy1 is localized in the nucleus of transiently transfected HEK293 cells (FIG. 1 panel C left and middle) and stably-infected NIH 3T3 cells (FIG. 1 panel C right) and it is tightly associated with an insoluble fraction which is highly enriched in histones (FIG. 3 panel B). The chromatin association of this protein is compatible with the results of recent studies showing that both Ndy1 and its homolog Ndy2 function as demethylases of histone H3 dimethylated at K36 or histone H3 trimethylated at K4 (Tsukada, Y. et al. (2006) Nature 439, 811-6; Frescas, D. et al. (2007) Nature 450, 309-13).

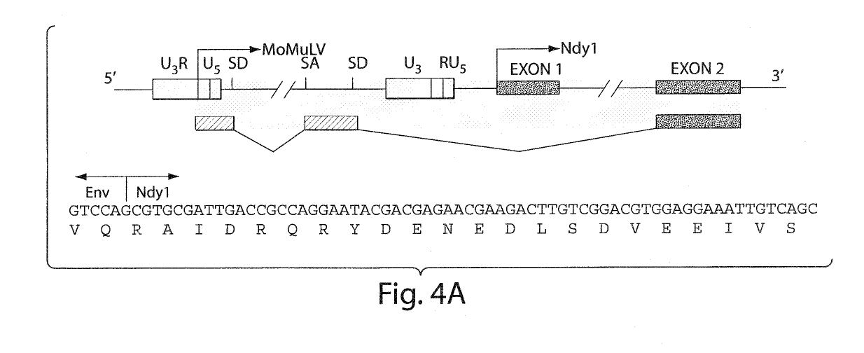

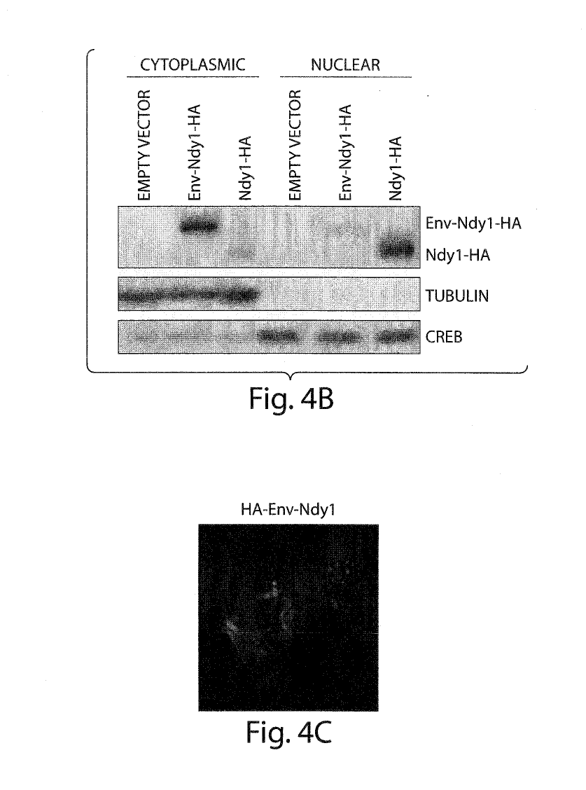

Northern and Western blotting of different rat tissues revealed that Ndy1 is expressed primarily in testis, spleen and thymus (FIG. 3 panel A). Moreover, Northern and Western blotting carried out on normal rat thymus and several MoMuLV-induced rat T cell lymphomas showed that Ndy1 expression is increased in all tumors that harbor a provirus in the Ndy1 locus. Highest levels of Ndy1 expression were detected in tumors in which the orientation of the integrated provirus was the same as the orientation of the Ndy1 gene (tumors Do and 2775; FIG. 1 panels D and E). However, these tumors also express high levels of aberrant mRNA transcripts encoding an Env-Ndy1 non-nuclear chimeric protein (FIG. 4).

Characterization of the aberrant transcripts in tumors with a provirus observed in tumors carrying a provirus in the same transcriptional orientation as the Ndy1 gene, revealed that they are generated by splicing between a cryptic splice donor site within the Env gene of the virus and the splice acceptor site at the start of exon 2. The cryptic splice donor site in the Env gene functions as the splice donor for the generation of virus-Mlvi-4 hybrid transcripts (Patriotis, C. et al. (1994) J Virol 68:7927). The Env-Ndy1 fusion protein encoded by these transcripts may be glycosylated and membrane-bound. Expression of this protein in HEK293 cells transiently transfected with a cDNA construct of the hybrid cDNA confirmed that it is not localized in the nucleus.

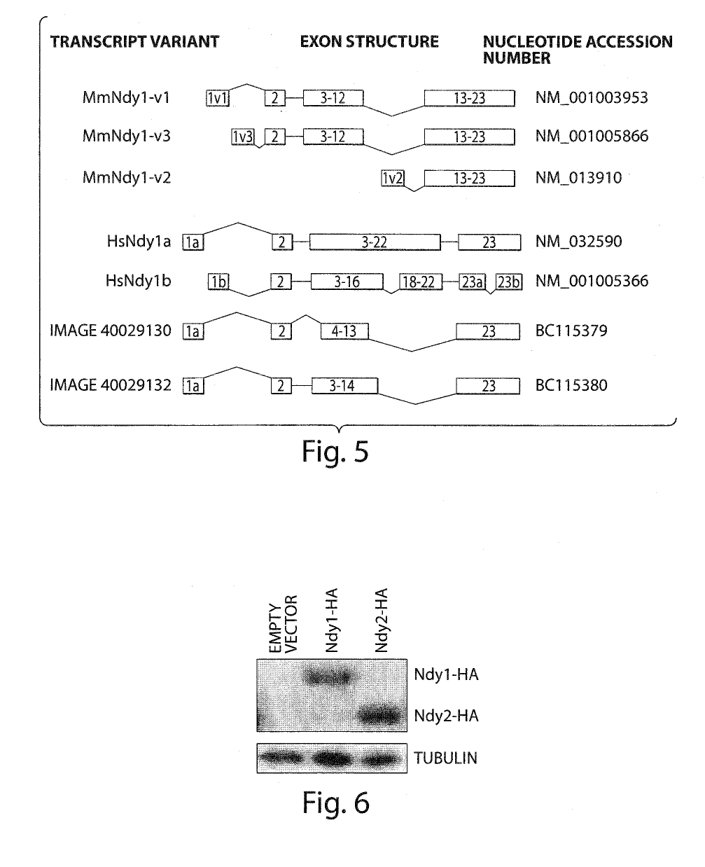

Sequence comparison of Ndy1 cDNA clones in the Genbank revealed that there are multiple forms of the Ndy1 mRNA that are generated through differential transcriptional initiation or alternative splicing. The protein predicted to be encoded by one of these transcripts lacks the JmjC domain and contains a unique N-terminal region (NM_{—}01390, FIG. 5). This protein is described herein as the short form of Ndy1, and Examples utilized the v1 variant of Ndy1 (NM_{—}001003953; SEQ ID NO: 31).

Analysis of available database information showed the existence of three isoforms of Ndy1 in mouse and rat. Mouse Ndy1 transcript variant 1 contains 23 exons, is identical to clone IMAGE 6851421 of the National Institutes of Health Mammalian Gene Collection (NIH-MGC) and encodes the mouse KIAA3014 protein. Transcript variant 3 also contains 23 exons but differs from transcript variant 1 in the first exon. Specifically, the 225 nt exon 1v1 of transcript variant 1 is replaced by the 66 nt exon 1v3 which is transcribed from an alternative promoter. The third isoform, transcript variant 2, was detected in mouse rat and human, is significantly shorter than the other two transcripts and contains 12 exons of which the first (exon 1v2) is unique to this transcript and the remainder, exons 13-23 are shared. The 776 as protein encoded by this transcript, contains the CXXC zinc finger motif, the F-box, and the LRR domain but lacks the JmjC domain.

Several variant transcripts have also been detected in humans. Transcript variants a and b, both have 23 exons. Due to differential transcriptional initiation, variant a contains a shorter 199 nt first exon (exon 1a), that is not homologous to the first exons of any of the known mouse variant transcripts, and variant b a longer 439 nt exon (exon 1b), that is homologous to the mouse exon 1v1. However, due to differences in the size of the 5′UTR, variant a encodes an N-terminus that is 31 as longer. Furthermore, exon 17 is not present in variant b, while variant a contains a longer last exon corresponding to the unspliced form of the last two exons of isoform b. The translational termination codon of the unspliced transcript is in the intron between exons 23a and 23b. As a result, a 17 amino acid sequence in the C-terminus of the protein encoded by the unspliced message is replaced by a 15 amino acid sequence in the protein encoded by the spliced message.

Additional human transcripts reported in existing databases include: Clone IMAGE 40029130 (SEQ ID NO: 36) of the NIH-MGC starts with exon 1a, continues to exon 2 (similar to variant a), bypasses exon 3 and continues with exons 4-13, and then jumps to exon 23. This gives rise to a protein that contains only the JmjC domain and the CXXC zinc finger. Clone IMAGE 40029132 (SEQ ID NO: 37) of the same consortium is similar to clone IMAGE 40029130 (SEQ ID NO: 36), but contains exons 3 and 14. The inclusion of exon 14 results in the incorporation of a part of the PHD-zinc finger domain into the protein.

The existence of multiple isoforms of Ndy1 is corroborated by the identification of 9 splicing isoforms in chimpanzee (Pan troglodytes) and 10 isoforms in dog (Canis familiaris). Using an antibody specific for a region of human Ndy1 immediately following the PHD zinc finger domain that is distinct from the related Ndy2, six isoforms were detected in HEK293 cells. This antibody recognized one major band at 97 kDa and five additional protein bands ranging from 88 to 172 kDa that were significantly depleted upon treatment with siRNAs against Ndy1 (Gearhart M. D., et al. (2006) Mol Cell Biol 26, 6880-92006), indicating that there remain more Ndy1 isoforms that need to be identified.

### Example 7

### MEFs Engineered to Express Exogenous Ndy1 Bypass Replicative Senescence and Undergo Immortalization

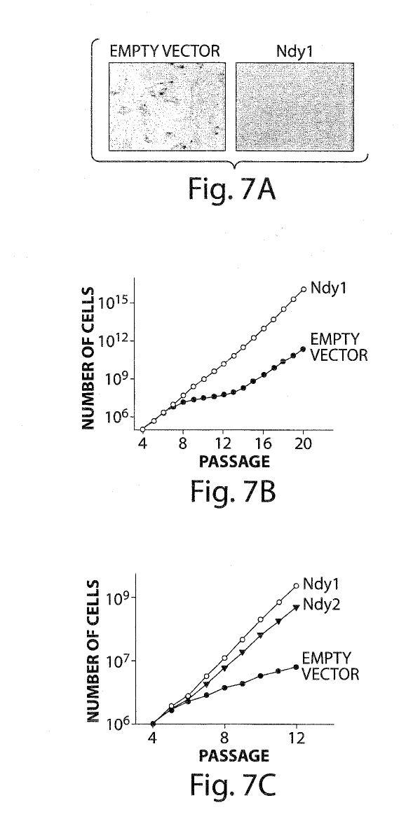

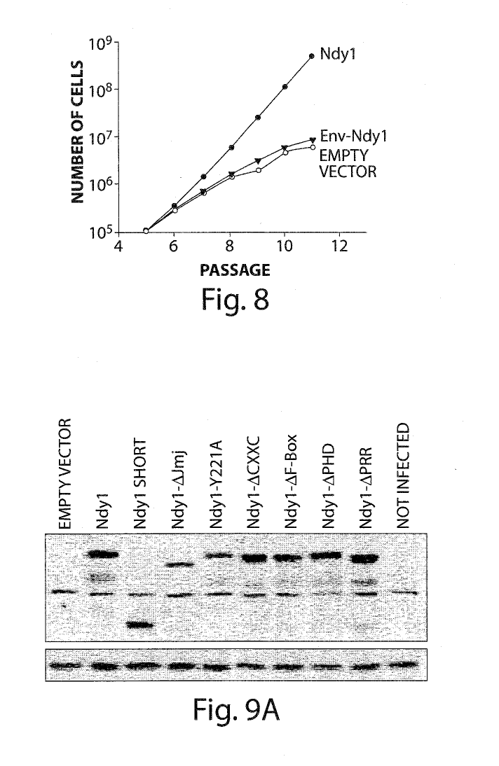

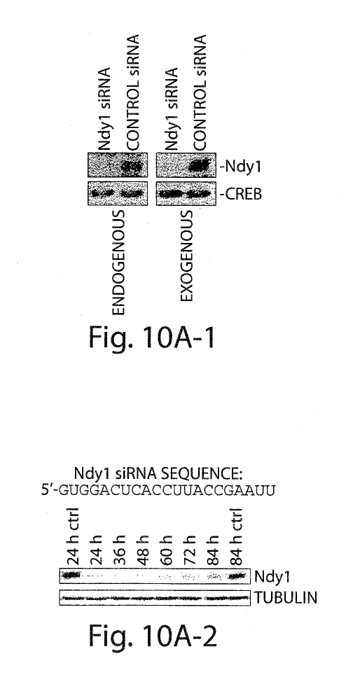

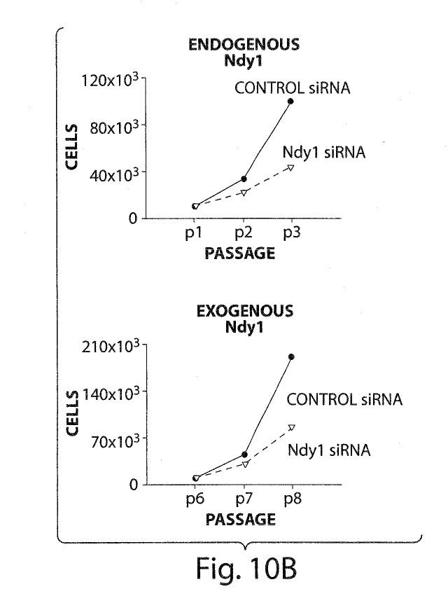

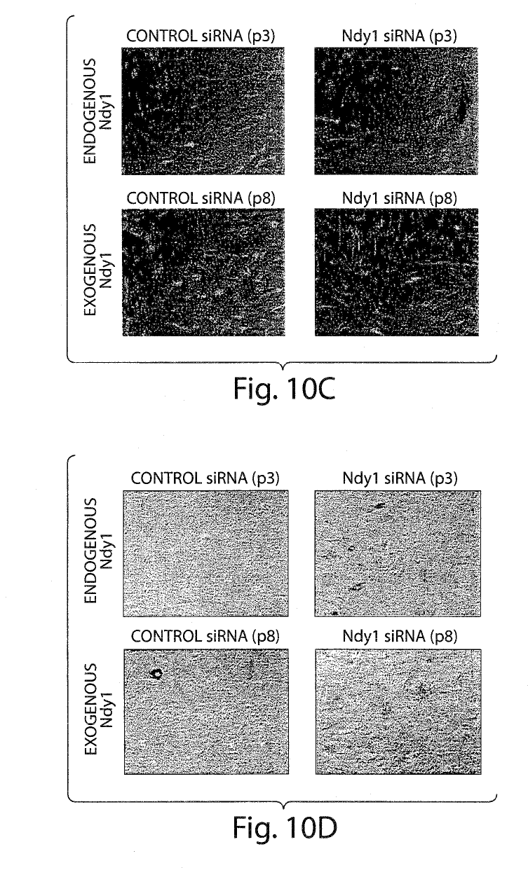

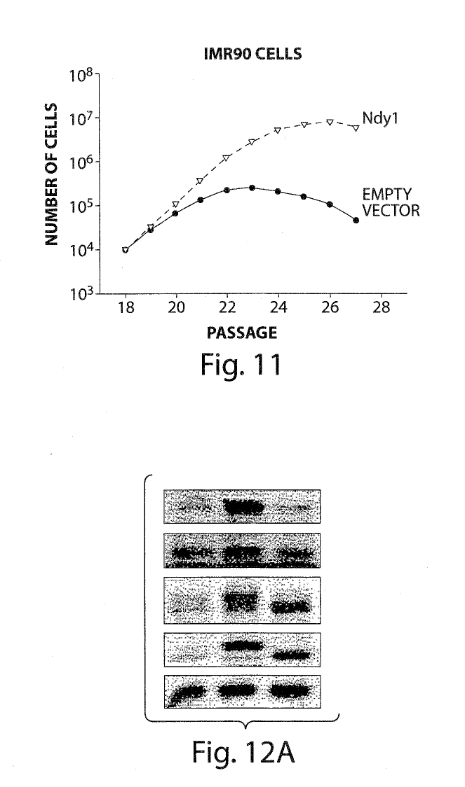

To determine the phenotypic effects of Ndy1 expression, MEFs were infected with an Ndy1-MigR1 retrovirus construct, or with the empty MigR1 retrovirus vector (FIG. 10). After several passages, the cells infected with the empty vector were observed to be senescing, while the MigR1-Ndy1-infected cells continued to divide without signs of senescence. Staining the cells at passage 11 for β-galactosidase (FIG. 2 panel A) confirmed this observation. Plotting the cumulative cell number at each passage (FIG. 2 panel B) revealed that whereas the proliferation of the vector-infected cells practically stops between passages 8 and 13, the proliferation of Ndy1-expressing cells continues uninterrupted. At the end, both the MigR1 and the MigR1-Ndy1-infected cells undergo immortalization. However, only the MigR1-Ndy1-infected cells bypass replicative senescence. Further studies revealed that not only Ndy1, but also Ndy2 promotes immortalization of MEFs in culture (FIG. 6 and FIG. 7 panel C). Moreover, passaging of cells infected with a MigR1-Env-Ndy1 retrovirus revealed that the cytoplasmic Env-Ndy1 protein does not immortalize MEFs, suggesting that the nuclear localization of the protein is required for the immortalization phenotype (FIGS. 8 and 11).

### Example 8

### The Histone Demethylase Activities of Ndy1 and Ndy2 are Required for Immortalization

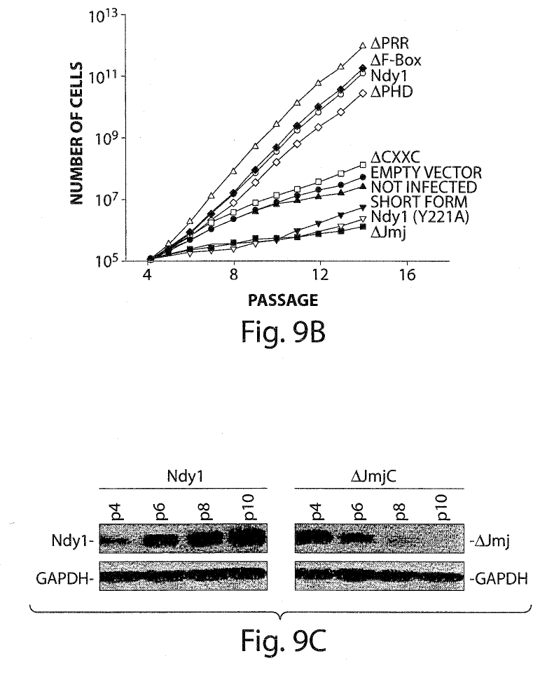

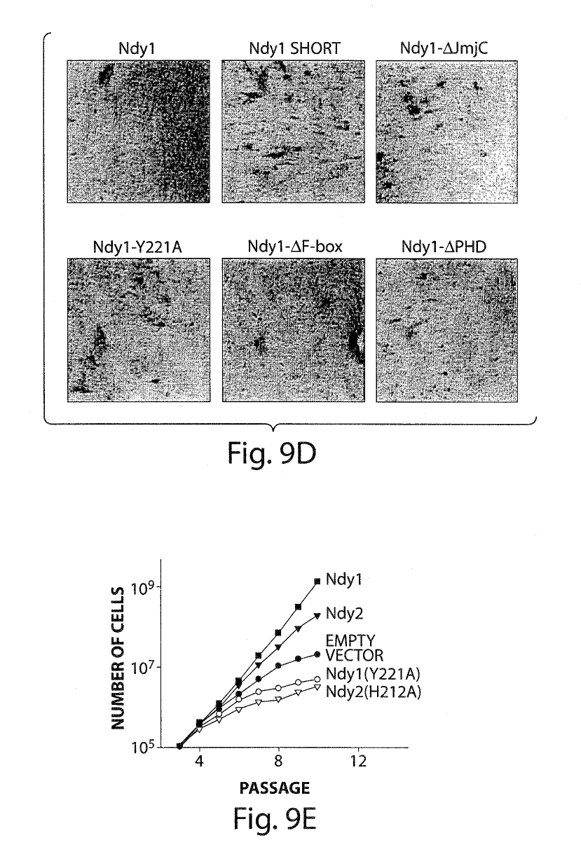

Ndy1 is a multi-domain protein that may exhibit multiple functional activities, which singly or in combination may contribute to the observed immortalization phenotype. To map the domain responsible for immortalization, the immortalizing activity of single domain deletion mutants of Ndy1 was addressed (FIG. 9 panel A). Regarding the JmjC domain, in addition to the deletion mutant, the physiologically expressed short form of Ndy1 which lacks the JmjC domain was used, as well as the point mutants Ndy1 Y221A and Ndy H283Y. The Y221A mutation was based on a similar mutation that inhibits the biological activity of the Saccharomyces pombe protein Epe1 (Ayoub, N. et al. (2003) Mol Cell Biol 23, 4356-70; Zofall, M. et al. (2006) Mol Cell 22, 681-92; Verdel, A. (2006) Mol Cell 22, 709-10; Trewick, S. C. et al. (2007) Embo J 26, 4670-4682), while the H283Y mutation modifies the Fe(II) binding pocket of the JmjC domain, which is associated with the demethylase activity.

Passaging the stably-infected MEFs separated them into three distinct groups that differed regarding the emergence of senescence. One group included non-infected and empty vector-infected cells, as well as cells infected with the CXXC mutant, which began to show evidence of senescence by passage 7 to 8.

The second group included cells infected with the ΔjmjC deletion mutant, the short form of Ndy1, and the two JmjC domain point mutants, which were observed to begin to show evidence of senescence at a very early passage, suggesting that endogenous Ndy1 inhibits senescence and that these mutants interfere with the physiological function of the endogenous protein.

The third group included cells infected with wild type Ndy1 and all the remaining mutants, which continued to immortalize MEFs (FIG. 9 panel B). These observations were further supported by two additional observations: a) Western blots of passaged MEFs infected with MigR1-Ndy1.HA or MigR1-ΔJmjC Ndy1.HA constructs, revealed that Ndy1.HA expression increases, while ΔJmjC Ndy1.HA expression decreases with each passage, indicating that Ndy1.HA expressing cells are positively selected and ΔJmjC Ndy1.HA-expressing cells are counter-selected (FIG. 9 panel C); and b) β-galactosidase staining of 11^{th} passage MEFs infected with wild type or mutant Ndy1, stained strongly only the cells infected with constructs of the JmjC domain mutants.

To determine whether MEF immortalization by Ndy2 is also JmjC domain-dependent, the preceding analysis was repeated with Ndy2, and the Ndy2 JmjC domain mutant H212A, which does not bind Fe(II) and has no demethylase activity (Tsukada, Y. et al. (2006) Nature 439, 811-6). Wild type Ndy1, the Ndy1 mutant Y221A and the empty vector were used as controls. The results also showed that MEF immortalization by Ndy2 was dependent on a functional JmjC domain (FIG. 9 panel E).

### Example 9

### Endogenous Ndy1 is a Physiological Inhibitor of Senescence

To determine whether endogenous Ndy1 is a physiological regulator of senescence, passage 3 wild type MEFs and passage-8 MEFs overexpressing Ndy1 were transfected with Ndy1 or control siRNA. The downregulation of both the endogenous and the exogenous Ndy1 in the transfected cells was observed by Western blotting (FIG. 10 panel A1 and FIG. 10 panel A2). The transfected cells were passaged twice, and cell proliferation was monitored by counting the cells at each passage. Cell morphology and β-galactosidase staining were recorded after the second passage (FIG. 10 panel B, FIG. 10 panel C and FIG. 10 panel D). The data confirmed that the endogenous Ndy1 indeed protects dividing cells from replicative senescence.

### Example 10

### Ndy1 does not Prevent Senescence in IMR90 Cells

To determine whether Ndy1 also promotes immortalization of primary human fibroblasts, IMR90 cells were infected with control vector MigR1 or with MigR1-Ndy1. After passage 20, the proliferation of control vector-infected IMR90 cells was observed to have slowed down, while the proliferation of MigR1-Ndy1-infected IMR90 cells continued, showing that Ndy1 inhibits early senescence in these cells. However the data further show that the proliferation of MigR1-Ndy1-infected cells also slowed down by passage 26, and the cells failed to undergo immortalization (FIG. 11).

Since human cells go into senescence because of telomere shortening (Blackburn, E. H. (2000) Nature 408, 53-6, Bodnar, A. G. et al. (1998) Science 279, 349-52), these data indicate that Ndy1 does not protect cells from telomere erosion, although it inhibits the DNA damage response elicited by the erosion (von Zglinicki, T. et al. (2005) Mech Ageing Dev 126, 111-7).

### Example 11

### Ndy1 Promotes MEF Immortalization by Targeting the Rb and p53 Pathways

The activation of the Ink4a/ARF locus and the response to DNA damage, which are the main factors promoting senescence, target both the p53 and Rb pathways (Genovese, C. et al. (2006) Oncogene 25, 5201-9, Sharpless, N. E. et al. (2002) Cell 110, 9-12, Sharpless, N. E. (2005) Mutat Res 576, 22-38).

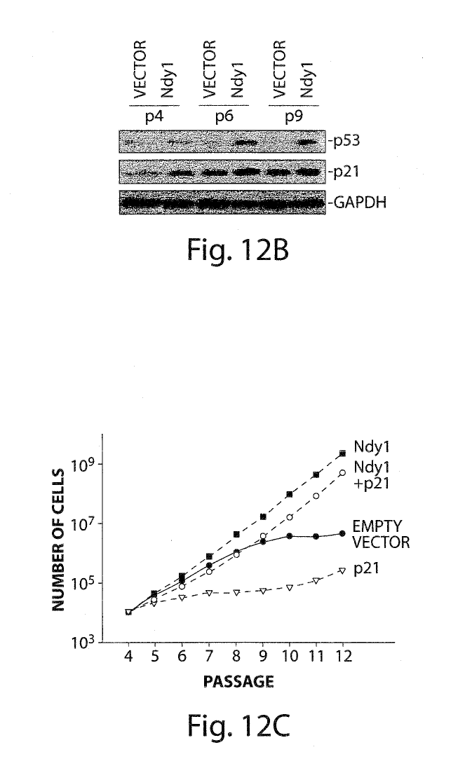

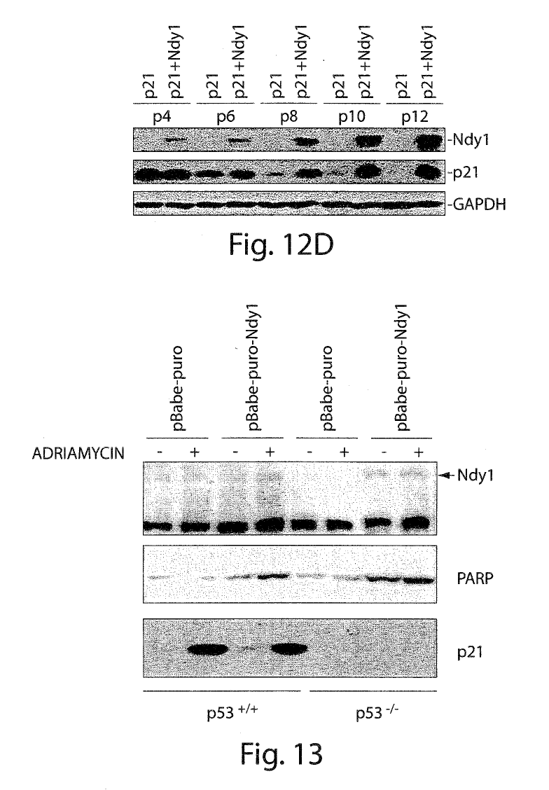

To determine which pathway may be targeted by Ndy1, the phosphorylation of Rb at Ser807/811 and Ser780 and the expression of p53 and its target p21^{CIP1} in early and late passage MEFs infected with MigR1 and MigR1-based constructs of Ndy1 and ΔJmjC-Ndy1 was examined. The results (FIG. 12 panel A) showed that Ndy1 selectively promotes the phosphorylation of Rb at Ser807/811. However, Ndy1 also upregulates p53 and its target p21^{CIP1} (Collado, M. et al. (2007) Cell 130, 223-33; FIG. 12 panel B). Overexpression of Ndy1 in p53^{+/+} and p53^{-/-} HCT116 cells, revealed that the induction of p21^{CIP1} by Ndy1 is p53-dependent (FIG. 13).

These data could be interpreted by the hypothesis that Ndy1 immortalizes cells by targeting the Rb pathway and that in the presence of Ndy1, the activation of the p53/p21^{CIP1} pathway does not inhibit cell proliferation. To address this hypothesis, MEFs were infected with a MigR1-GFP construct of Ndy1, a MigR1-RFP construct of p21^{CIP1}, a combination of the two, or with the MigR1-GFP empty vector control. Passaging revealed that cells overexpressing p21^{CIP1} senesced rapidly, while cells overexpressing both p21^{CIP1} and Ndy1 immortalized nearly as efficiently as cell overexpressing only Ndy1 (FIG. 12 panel C). Probing Western blots of cell lysates harvested at the indicated passages with antibodies specific for each of p21^{CIP1} or Ndy1, revealed that, in the absence of Ndy1, p21^{CIP1}-overexpressing cells were strongly counter-selected. However, in the presence of Ndy1, they were positively selected (FIG. 12 panel D), showing that Ndy1 expression indeed abrogates the cell cycle inhibitory activity of p21^{CIP1}.

In Examples herein, it was observed that Ndy-overexpressing cells bypass replicative senescence and undergo immortalization via a JmjC domain-dependent process. In addition, knocking down Ndy1 and expression of dominant negative mutants of Ndy1 were observed to promote senescence, indicating that Ndy1 is a physiological inhibitor of senescence in dividing cells. Ndy2, a homolog of Ndy1, also promotes immortalization of MEFs. Immortalization was linked to the selective phosphorylation of Rb (Genovese, C. et al. (2006) Oncogene 25, 5201-9) and the selective abrogation of the pro-senescence activity of p21^{CIP1} (Sharpless, N. E. et al. (2002) Cell 110, 9-12; Choudhury, A. R. et al. (2007) Nat Genet 39, 99-105).

Ndy1 and Ndy2 are multi-domain chromatin-associated proteins (Tsukada, Y. et al. (2006) Nature 439, 811-6; Klose, R. J. et al. (2006) Nat Rev Genet 7, 715-27). Systematic deletion of all the known domains of Ndy1 revealed that the JmjC and the CXXC domains are the only ones required for immortalization. However, whereas the JmjC domain mutants exhibit a dominant negative, pro-senescence phenotype, the CXXC motif mutants do not. These findings indicated that the JmjC domain provides the functional activity for immortalization and that the CXXC motif controls immortalization by regulating the DNA targeting of the protein. In the absence of the correct DNA binding, the function of the protein is impaired. However, the mutant does not have a dominant negative phenotype because it does not interfere with function of the endogenous protein (Frescas, D. et al. (2007) Nature 450, 309-13). Further, the Env-Ndy1 hybrid protein, which is localized primarily in the cytoplasm, also lacks both immortalizing and dominant negative pro-senescence activities.

Ndy1 and Ndy2 possess JmjC domain-dependent histone H3 demethylase activities. Ndy2, and perhaps Ndy1, demethylate histone H3 dimethylated at K36 (Tsukada, Y. et al. (2006) Nature 439, 811-6). Moreover, Ndy1 demethylates histone H3 trimethylated at K4 (Frescas, D. et al. (2007) Nature 450, 309-13). JmjC domain-dependent demethylation is an oxidative reaction that requires Fe(II) and a-ketoglutarate as co-factors (Tsukada, Y. et al. (2006) Nature 439, 811-6). The JmjC domain residues that coordinate the binding of these cofactors have been mapped (Klose, R. J. et al. (2006) Nat Rev Genet 7, 715-27). Mutation of some of these sites in Ndy1 and Ndy2 gave rise to proteins that exhibited dominant negative pro-senescence, rather than immortalizing phenotypes, indicating that the historic demethylase activities of these proteins are required for immortalization.

Cellular senescence is due to a number of factors, including the progressive shortening of telomeres, the activation of the Ink4a/ARF locus and telomere shortening independent DNA damage (Collado, M. et al. (2007) Cell 130, 223-33). MEFs and human fibroblasts differ with regard to the relative importance of telomere shortening in the induction of senescence in culture. Thus, the primary cause of senescence in human, but not in mouse fibroblasts is telomere erosion (Blackburn, E. H. (2000) Nature 408, 53-6; Bodnar, A. G. et al. (1998) Science 279, 349-52), which is recognized as DNA damage (von Zglinicki, T. et al. (2005) Mech Ageing Dev 126, 111-7). Given that Ndy1 prevented early senescence but failed to immortalize IMR90 cells, data herein show that it interferes with the withdrawal from the cell cycle induced by telomere shortening, but it does not prevent telomere shortening per se.

The shortening of telomeres and the activation of the Ink4a/ARF locus may be developmentally programmed in dividing cells (Collado, M. et al. (2007) Cell 130, 223-33). In addition however, these processes can be induced in response to DNA damage, which plays a central role in the progression into senescence (von Zglinicki, T. et al. (2005) Mech Ageing Dev 126, 111-7; Blasco, M. A. (2005) Nat Rev Genet 6, 611-22). In dividing cells, the DNA damage response is activated by telomere shortening, oxidative stress, the aberrant firing of replication origins, or by activated oncogenes (Collado, M. et al. (2007) Cell 130, 223-33). Signaling pathways activated by DNA damage target the Rb and p53 pathways and induce reversible or irreversible cell cycle arrest or apoptosis (Genovese, C. et al. (2006) Oncogene 25, 5201-9, Sharpless, N. E. et al. (2002) Cell 110, 9-12; Sharpless, N. E. (2005) Mutat Res 576, 22-38). To determine therefore the mechanism by which Ndy1 prevents senescence, the effects of its overexpression on the Rb and p53 pathways were examined. In examples herein results showed that Ndy1 promotes the phosphorylation of Rb at Ser807/811. Since phosphorylation at this and other sites relieves the transcriptional repression activity of Rb and promotes progression through the G1 phase of the cell cycle, these data provide an explanation for the immortalizing activity of Ndy1.

### Example 12

### Replicative Senescence in MEFs is Associated with the Downregulation of Ndy1, and to a Lesser Extent Ndy2

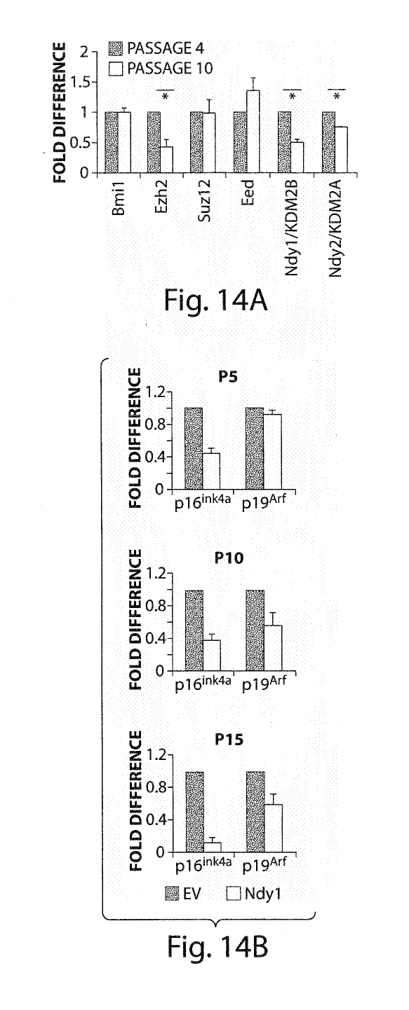

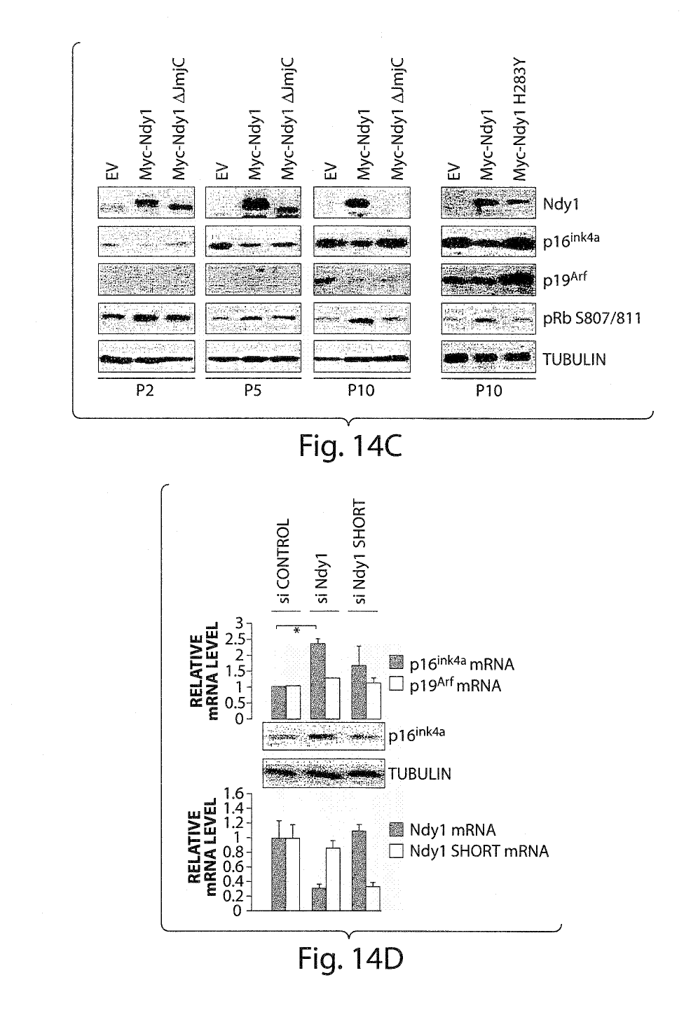

Examples above show that Ndy1 functions as a physiological inhibitor of senescence in MEFs (Pfau, R., et al. (2008) Proc Natl Acad Sci USA 105, 1907-12 incorporated herein in its entirety). That Ndy1 is downregulated in cells undergoing replicative senescence is shown by the results in FIG. 14 panel A, that indeed Ndy1 and to a lesser degree Ndy2 were downregulated in passaged MEFs and that their downregulation was passage-dependent.

RNA was isolated with the RNeasy mini kit (Qiagen #74104). cDNAs of these mRNAs were synthesized with RETROscript (Ambion #AM1710). PCR reactions were carried out in triplicate with iQ SYBRGreen Supermix (Bio-Rad #170-8882) in an Opticon2 continuous fluorescence detector (MJ Research). The following set of primers have been used to amplify the mouse: Ezh2: F-5′-ACTGCTGG CACCGTCTGATG-′3 (SEQ ID NO: 38) and R-5′-TCCTGAGAA ATAATCTCCCCACAG-′3 (SEQ ID NO: 39),

### Bmi1: F-5′-AGATGAGTCA CCAGAGGGATGG-′3 (SEQ ID NO: 40) and R-5′-TCACTCCCAG AGTCACTTTCCAG-′3 (SEQ ID NO: 41),

p16^{Ink4a}: F-5′-GTGTGCATGA CGTGCGGG-′3 (SEQ ID NO: 42) and R-5′-GCAGTTCGA ATCTGCACCGTAG-′3 (SEQ ID NO: 43),
p19^{Arf}: F-5′GCTCTGGCT TTCGTGAACATG-′3 (SEQ ID NO: 44) and R-5′-TCGAATCTG CACCGTAG′TTGAG'3 (SEQ ID NO: 45),

### Ndy1: F-5′-AGACACCAGA GGCACAGAGG-3′ (SEQ ID NO: 25) and R-5′-CACAGTGGG ACGCTTGACTA-3′ (SEQ ID NO: 46),

### GAPDH: F-5′-TGTGTCCG TCGTGGATCTGA-3′ (SEQ ID NO: 27) and R-5′CCTGCTTCAC CACCTTCTTGA-3′ (SEQ ID NO: 28),

### Eed: F-5′-TGGCCATG GAAATGCTATCA (SEQ ID NO: 47) and R-5′-ACACCTCCG AATATTGCCACA-′3 (SEQ ID NO: 48),

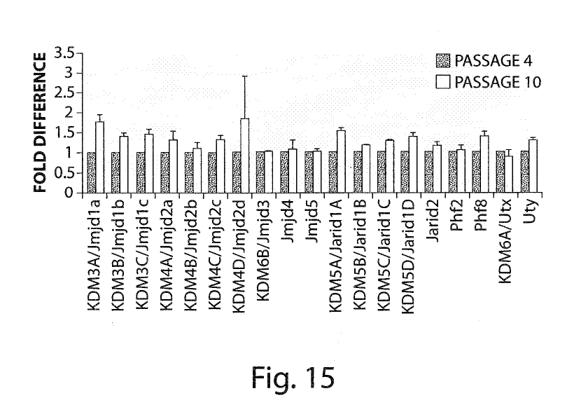

Suz12: F-5′-ACTATTGCTGTT AAGGAGACGCTGA-′3 (SEQ ID NO: 49) and R-5′-GCAGGTCGTC TCTGGCTTCT-′3 (SEQ ID NO: 50). The primers used to measure the mRNA levels of human p16^{Ink4a}, Ezh2, Suz12, Eed, and Bmi1 have been previously described (Bracken, A. P., et al. (2007) Genes Dev 21, 525-30). The primers used to measure the mRNA levels of different demethylases have been previously described (De Santa, F., et al. (2007) Cell 130, 1083-94). Data were analyzed by using an Opticon2 continuous fluorescence detector (MJ Research) as previously described (Pfau, R., et al. (2008) Proc Natl Acad Sci USA 105, 1907-12, incorporated herein by reference in its entirety). Ezh2, which is known to be downregulated, and Bmi1, Suz12, and Eed1, which are known not to be downregulated during senescence were used as controls. The expression of other histone demethylases also was not downregulated (FIG. 15), indicating an important role of Ndy1 and Ndy2 in the physiological regulation of replicative senescence.

### Example 13

### Ndy1 Represses the Expression of p16^{Ink4a} and p19^{Arf} in MEFs

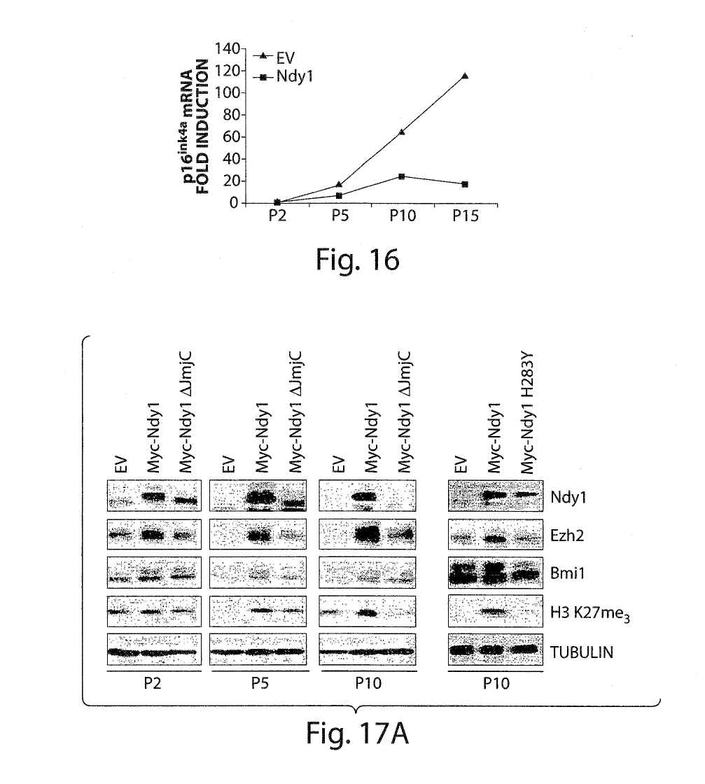

Replicative senescence is characterized by the dramatic upregulation of p16^{Ink4a} (>100-fold, FIG. 16) and p19^{Arf} (>10-fold). Since, p16^{Ink4a} inhibits the cyclin D/CDK4-6 complex that mediates the phosphorylation of Rb at Ser807/811, and Ndy1 promotes the phosphorylation of Rb at this site as shown in Examples herein, whether the phosphorylation of Rb in Ndy1-transduced MEFs can be linked to a defect in the induction of the Ink4a/Arf locus was tested. To address this question, the relative levels of p16^{Ink4a} and p19^{Arf} mRNA in passaged MEFs stably expressing Ndy1 were determined in examples herein.

Cells for protein extraction and Western blotting, were washed twice in ice-cold PBS and solubilized in the lysis buffer (50 mM Tris (pH 7.5), 200 mM NaCl, 1% Triton X-100, 0.1% SDS, 10 mM Na3VO4, 50 mM NaF, 1 mM β-glycerophosphate, 1 mM sodium pyrophosphate, 1 mM EDTA, 1 mM EGTA, and 1 mM PMSF, supplemented with a mixture of protease inhibitors). The lysates were sonicated in a Misonix 3000 sonicator for 5 seconds at power level 1.5, and they were centrifuged for 20 min at 16,000×g. The supernatant (soluble whole-cell lysate) was analyzed by Western blotting. The Ezh2 (#4905) and Rb Ser807/811 (#9308) antibodies were from Cell Signaling. The Bmi1 (#sc-10745), mouse p16^{Ink4a} (# sc-1207), human p16^{Ink4a} (#sc-759), p19^{Arf} (#sc-22784) antibodies were obtained from Santa Cruz Biotechnologies. The Bmi1 (#05-637) antibody was obtained from Millipore. The Ring1b (# ab3832) antibody was from Abcam. The Ndy1 antibody is described herein and in Pfau, R., et al. (2008) Proc Natl Acad Sci USA 105, 1907-12, incorporated herein by reference in its entirety.

FIG. 14 panels B and C and FIG. 16 show that overexpression of Ndy1 attenuated the induction of p16^{Ink4a} in passage 5, 10, and 15 by about 55%, 62% and 90%, respectively, relative to empty vector transduced MEFs. The overexpression of Ndy1 also attenuated the induction of p19^{Arf}, in later passages (about 43% and 34% reduction at passage 10 and 15, respectively). The expression of p16^{Ink4a} inversely correlated with the phosphorylation of Rb at Ser807/811 (FIG. 14 panel C) suggesting a causative relationship between the attenuated induction of p16^{Ink4a} and the enhanced phosphorylation of Rb in cells overexpressing Ndy1. Knock down of endogenous Ndy1 in passage 3 MEFs increased the expression of p16^{Ink4a}, by about 2.3 times (FIG. 14 panel D). These data show that Ndy1 is indeed a physiological repressor of p16 Ink4a

The JmjC domain deletion mutant (ΔJmjC) and the H283Y point mutant of Ndy1 failed to immortalize MEFs and they were counterselected during passage (FIG. 14 panel C and Examples above). Further, the same mutants failed to attenuate the induction p16^{Ink4a} and p19^{Arf} (FIG. 14 panel C) showing that the demethylase activity of Ndy1, which is required for the immortalization is also required for the repression of the Ink4a/Arf locus. Examples above show that Ndy1 encodes multiple protein isoforms in both humans and mice. One of these, the short form of Ndy1 (NM_{—}013910; SEQ ID NO: 33), lacks the JmjC domain and its overexpression promotes senescence. Knocking down the short form did not upregulate significantly either p16^{Ink4a} or p19^{Arf} (FIG. 14 panel D) further showing the importance of the JmjC domain in the regulation of the Ink4a/Arf locus.

### Example 14

### Ndy1 Upregulates Ezh2 and H3K27 Tri-Methylation

Cells undergoing replicative senescence downregulate Ezh2, the catalytic subunit of PRC2 (Kamminga, L. M., et al. (2006) Blood 107, 2170-9; Bracken, A. P., et al. (2007) Genes Dev 21, 525-30). Since PRC2 is required for the PRC1-mediated repression of the Ink4a/Arf locus, its downregulation promotes the transcriptional activation of the locus (Kamminga, L. M., et al. (2006) Blood 107, 2170-9; Bracken, A. P., et al. (2007) Genes Dev 21, 525-30). Ndy1 is also downregulated in MEFs during passage (FIG. 14 panel A). Further examples herein examined whether Ndy1 regulates the expression of Ezh2.

For siRNA. MEFs transfected with Ndy1 (1433-GUGGACUCACCUUACCGAAUU-1454; SEQ ID NO: 51) or control siRNA (Dharmacon) were plated at 10⁵ cells per 6 cm dish and 3-4 days later were harvested and analyzed (Pfau, R., et al. (2008) Proc Natl Acad Sci USA 105, 1907-12, incorporated herein by reference in its entirety). MEFs were transfected with the help of Lipofectamine 2000 (Invitrogen). To specifically knock down the short form of Ndy1 the following siRNA were used: 5′-CCGAGGACGACGACUAUGAUU-′3 (SEQ ID NO: 52) which specifically targets the exon1 of the v2 isoform of Ndy1 (NM_{—}013910). siRNAs for Ezh2 (a mix of two siRNAs form Applied Biosystems, #AM16708 siID #157427 and #157426) and Bmi-1 (Santa Cruz Biotechnologies, # sc-29815) were used in a final concentration of 80 nM.

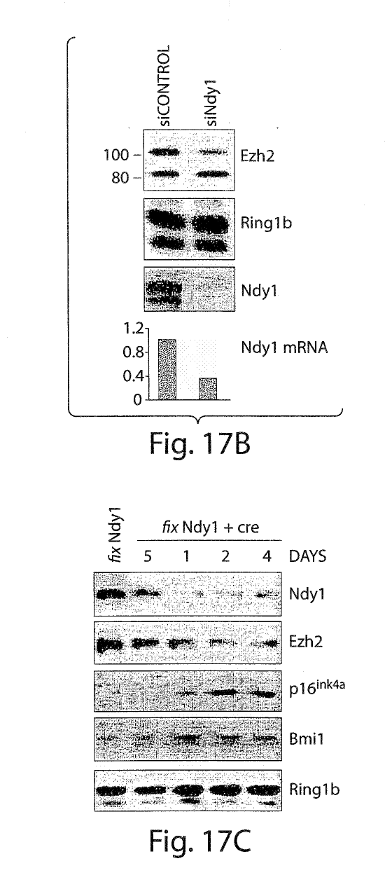

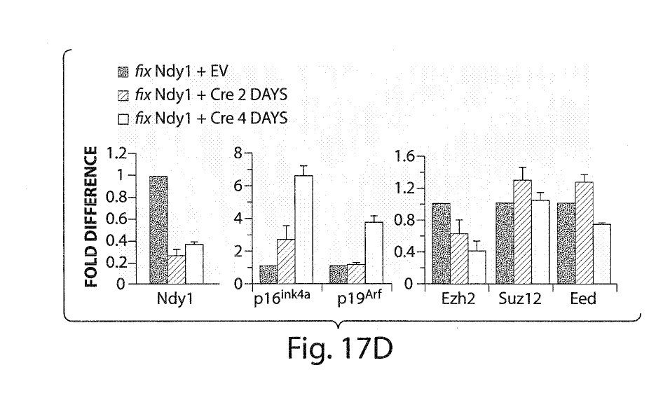

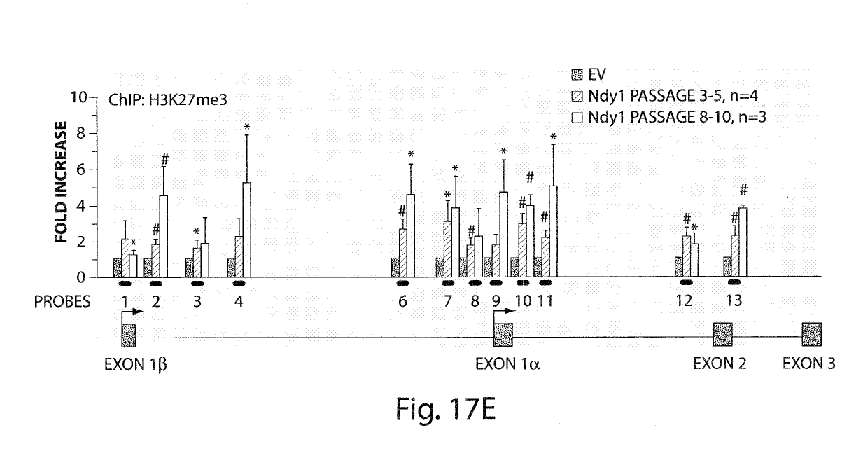

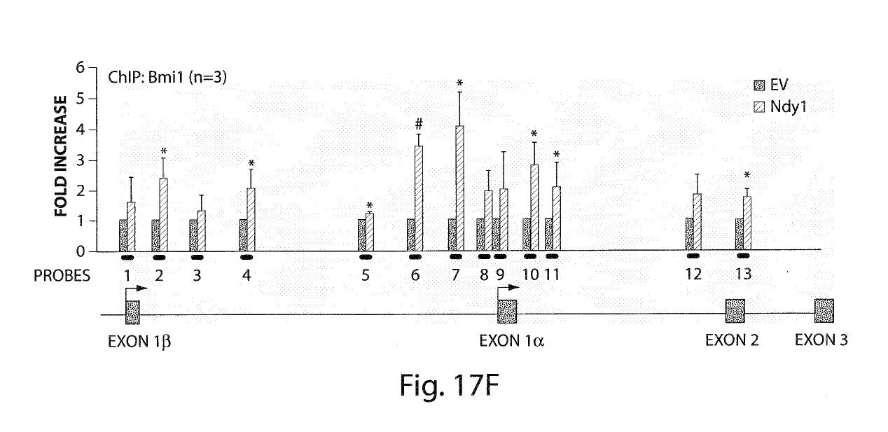

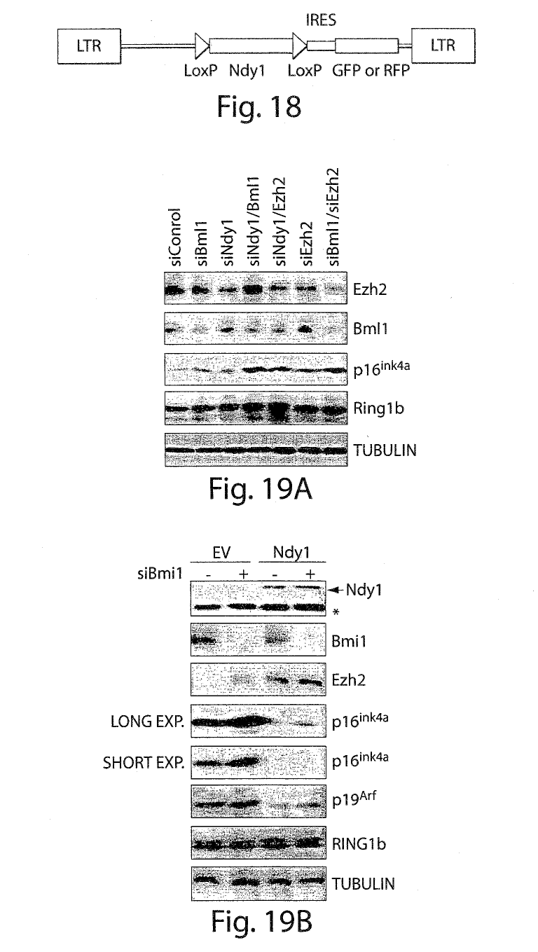

FIG. 17 panel A shows that overexpression of Ndy1 in MEFs promotes both the marked induction of Ezh2 and the global increase of histone H3 K27 tri-methylation, as early as the second passage after infection. Further, knocking down the endogenous Ndy1 in MEFs decreases the expression of Ezh2 (FIG. 17 panel B). The regulation of Ezh2 by Ndy1 was confirmed in MEFs immortalized with a foxed retroviral construct of Ndy1 (FIG. 18).

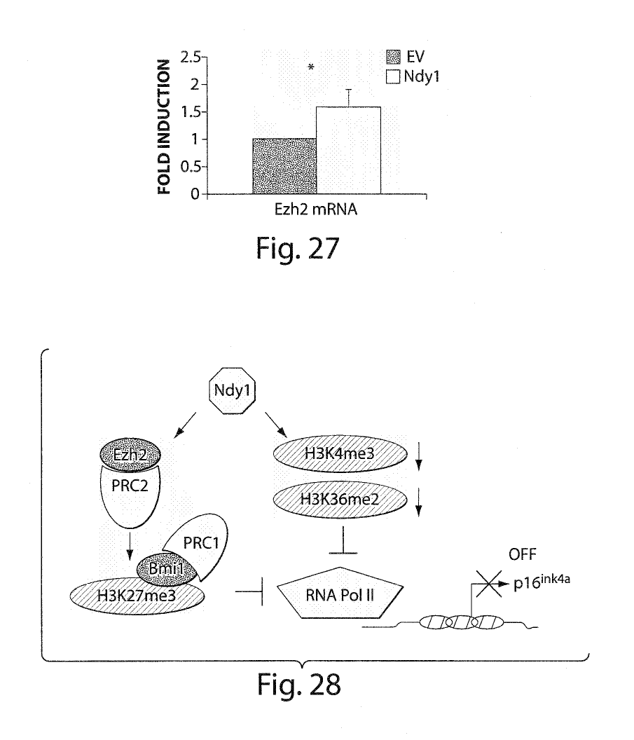

MigR1-LoxP-Ndy1-immortalized MEFs were infected with a retrovirus expressing the Cre recombinase. Cre-mediated excision of Ndy1 downregulates the Ndy1 protein (FIG. 17 panel C) and mRNA (FIG. 17 panel D) levels showing that exogenous Ndy1 was efficiently ablated. Ndy1 ablation resulted in a dramatic upregulation of p16^{Ink4a} and in a lesser upregulation of p19^{Arf}, and confirmed that Ndy1 represses the Ink4a/Arf locus. Finally, whereas excision of Ndy1 downregulated the mRNA and protein levels of Ezh2 (FIG. 17 panels C and D), overexpression of Ndy1 upregulated the mRNA levels of Ezh2 in MEFs and IMR90 cells (FIGS. 26 and 27), showing that Ndy1 upregulates Ezh2.

K27 tri-methylation of histone H3 has been linked to transcriptional repression (Cao, R., et al. (2002) Science 298, 1039-43; Bracken, A. P., et al. (2007) Genes Dev 21, 525-30; Bracken, A. P., et al. (2006) Genes Dev 20, 1123-36) and may therefore be responsible for the repression of the Ink4a/Arf locus. Chromatin immunoprecipitation assay (ChIP) was performed with a commercially available ChIP assay kit (Millipore; 17-295) following the manufacturer's instructions. MEFs from two 20 cm dishes were fixed with 1% formaldehyde for 10 min followed by two washes with PBS. Cells were lysed in 500 μl of SDS lysis buffer (50 mM Tris-HCl, pH 8.0, 1% SDS, 150 mM NaCl, and 5 mM EDTA plus protease inhibitor cocktail I from Roche #11836170001), and were incubated on ice for 10 min followed by sonication 3 times for 10 seconds each with 10 seconds off interval times at output setting 3 with a Misonix Sonicator 3000 to achieve a chromatin size of 200-700 bp.

The sonicated lysates were centrifuged at 14.000×g for 30 min at 4° C. and diluted 10 times with dilution buffer (16.7 mM Tris, pH 8.0, 167 mM NaCl, 0.01% SDS, 1.1% Triton X-100, 1.2 mM EDTA, Millipore, #20-153). DNA was recovered from immune complexes on protein A- or G-agarose beads with the following antibodies: H3K27me3 (#ab6002-100; Abeam), Myc clone 9B11 (#2276; Cell Signaling), H3K4me3 (#9751; Cell Signaling), H3K36me2 (#07-274; Millipore), RNA Polymerase II (# sc-899; Santa Cruz Biotechnologies) and Bmi1 (#05-637, Millipore) overnight at 4° C. on a rocking platform. Subsequently, the beads were washed once with Low Salt Immune Complex Wash Buffer (20 mM Tris-HCl, pH 8.0, 150 mM NaCl, 0.1% SDS, 1% Triton X-100, 2 mM EDTA, Millipore #20-154), followed by High Salt Immune Complex Wash Buffer ((20 mM Tris-HCl, pH 8.0, 500 mM NaCl, 0.1% SDS, 1% Triton X-100, 2 mM EDTA, Millipore #20-155), LiCl Immune Complex Wash Buffer (10 mM Tris-HCl, pH 8.0, 0.25 M LiCl, 1% NP40, 1% sodium deoxycholate, 1 mM EDTA, Millipore #20156), and twice with Tris-EDTA. DNA-protein cross-links were eluted with elution buffer (0.1 M NaHCO3 and 1% SDS) at room temperature for 30 min. After adjusting NaCl concentration, cross-linking was reversed with overnight incubation at 65° C. followed by proteinase K treatment for 1 hour at 45° C. The immunoprecipitated DNA was recovered by a PCR purification Kit (Qiagen #28106). Real-time PCR took place with iQ SYBRGreen Supermix (Bio-Rad #170-8882) to a final volume of 25 μl in Opticon2 continuous fluorescence detector (MJ Research).

Data are presented as the fold difference between the empty vector and Ndy1 overexpressing MEFs from at least 3 independent analyses, each one performed in triplicate. The primers used in ChIP assays for the amplification of the mouse Ink4a/Arf locus (chromosome 4, strain C57BL/6J, locus #NT_{—}039260, accession number # NT_{—}039260.7) were the following: Set1 (nucleotide position #28568975): F-5′-AAAACCCTCT CTTGGAGTGGG-′3 (SEQ ID NO: 53) and R-5′-GCAGGTTCTT GGTCACTGTGAG-′3 (SEQ ID NO: 54),

### Set2 (#28568312): F-5′-CTCCCTTTGCT ACCCCTGAGAG-′3 (SEQ ID NO: 55) and R′-TTACTTATTTC GCTCCCATCCAC-′3 (SEQ ID NO: 56),

### Set3 (#28566969): F5′-CTTAGAGTTAC AGAAAGGGCTGGA-′3 (SEQ ID NO: 57) and R-5′-GAATTTCAAGG AAGTGCTACCCTA-′3 (SEQ ID NO: 58),

### Set4 (#28565557): F-5′-GTTTCAGGAA AGCCAAACCA-′3 (SEQ ID NO: 59) and R-5′-GGTAGCCCA GGGTACTGTGA-′3 (SEQ ID NO: 60),

### Set5 (#28562284): F-5′AAGGGTCAACT GTCCTGTGG-′3 (SEQ ID NO: 61) and R-5′-GAAGATACTG AGGCCCACCA-′3 (SEQ ID NO: 62),

### Set6 (#28560624): F-5′-CACTGCACTGGAAGAGGACA-′3 (SEQ ID NO: 63) and R-5′CTGAAGGTCCTGGGTTCAAA-′3 (SEQ ID NO: 64),

### Set7 (#28557671): F-5′-TTCTGAGTTTA TGCTGAGTTCCAG-′3 (SEQ ID NO: 65) and R-5′-GTACTGGACAGA AGGGAGGATTTA-′3 (SEQ ID NO: 66),

### Set8 (#28556888): F-5′-GATGGAGCCCG GACTACAGAAG (SEQ ID NO: 67) and R-5′-CTGTTTCAAC GCCCAGCTCTC-′3 (SEQ ID NO: 68),

### Set9 (#28556465): F-5′-CAAAAGTTAC CCGACTGCAGATG'3 (SEQ ID NO: 69) and R-5′-AAAAGAACATC GGTTTCAACTTGAC-′3 (SEQ ID NO: 70),

### Set10 (#28556052): F-5′TGTTGCAGTTT CAGAAGGCACC-′3 (SEQ ID NO: 71) and 5′-GAACTCTTTCG GTCGTACCCC-′3 (SEQ ID NO: 72),

### Set11 (#28555857): F-5′-GCTGAGAAGT TTGCCTTTGG-′3 (SEQ ID NO: 73) and R-5′AACTTCCTCC TTCCCCGTTA-′3 (SEQ ID NO: 74),

### Set12 (#28551696): F-5′-AGGGAATACA CTGTAAGCCTGTGT-′3 (SEQ ID NO: 75) and R-5′-TTAACTACTCG GATCAGACATCCA-′3 (SEQ ID NO: 76),

Set13 (#28551168): F-5′-CCCAGGTGA GCATAGTTGGT-′3 (SEQ ID NO: 77) and R-5′-GGGTGGGTAA AATGGGAACT-′3 (SEQ ID NO: 78). Probe #1 binds within the exon1β. The probes #7-9, #10, and #11, bind within the Ink4a promoter, at the end of exon1α, and 400 bp downstream of exon1α, respectively (Collado, M., et al. (2007) Cell 130, 223-33). The nucleotide distance between the different probes is: Set 1-2: 663 nt, Set 2-3: 1343 nt, Set3-4: 1412 nt, Set 4-5: 3273 nt, Set 5-6: 1660 nt, Set 6-7: 2953 nt, Set 7-8: 783 nt, Set 8-9: 423 nt, Set 9-10: 413 nt, Set 10-11: 195 nt, Set 11-12: 4161 nt, Set12-13: 528 nt.

Chromatin immunoprecipitation (ChIP) analyses herein indeed revealed that Ndy1 overexpression upregulated histone H3 K27 tri-methylation throughout the Ink4a/Arf locus (FIG. 17 panel E). The upregulation was particularly prominent in the promoter and coding regions of exon 1α (FIG. 17 panel F, probes 7-11) and to a lesser extent exons 1β, 2 and 3 (FIG. 17 panel F, probes 1-2, 12-13). The increase in H3K27 tri-methylation at the Ink4a/Arf locus was passage-dependent and correlated with the Ndy1-driven upregulation of Ezh2 and the global H3K27 tri-methylation (FIG. 17 panel A).

### Example 15

### Ndy1-Driven Histone Modifications in the Ink4a/Arf Locus Promote the Binding of Bmi1

Histone H3 K27 tri-methylation serves as a docking site for the binding of the chromodomain protein Polycomb (Pc), a component of PRC1 which represses the Ink4a/Arf locus in a Bmi1-dependent manner (Cao, R., et al. (2002) Science 298, 1039-43; Bracken, A. P., et al. (2007) Genes Dev 21, 525-30; Bracken, A. P., et al. (2006) Genes Dev 20, 1123-36; Hernandez-Munoz, I., et al. (2005) Mol Cell Biol 25, 11047-58). Ezh2 downregulation in cells undergoing senescence decreases histone H3 K27 tri-methylation at the Ink4a/Arf locus and leads to the displacement of PRC1-Bmi1 complex with subsequent activation of transcription. The results of ChIP analysis show that overexpression of Ndy1 increased the binding of endogenous Bmi1 to the promoter region and exon 1α of the Ink4a/Arf locus (FIG. 17 panel F, probes 6, 7, 10, and 11). Of note, the binding of Bmi1 to this locus exhibited a similar pattern to that of histone H3 K27 tri-methylation (FIG. 17 panel E), supporting the conclusion that the Ndy1-induced Ezh2-dependent H3K27 tri-methylation functions as a priming event for the binding of Bmi1, a component of PRC1, and the repression of p16^{Ink4a}.

### Example 16

### Ndy1 Cooperates with Ezh2 and Bmi1 to Repress p16^{Ink4a}

Ezh2 overexpression, histone H3 K27 tri-methylation, and Bmi1 binding to the Ink4a/Arf locus were observed herein to immortalize MEFs by repressing p16^{Ink4a}. This raised the question whether Ezh2 upregulation and Bmi1 binding to the Ink4a/Arf locus are sufficient for the Ndy1 immortalization phenotype. To address this question, each of Ndy1, Bmi1, or Ezh2 alone and in combinations were transiently knocked down in passage 2 MEFs.

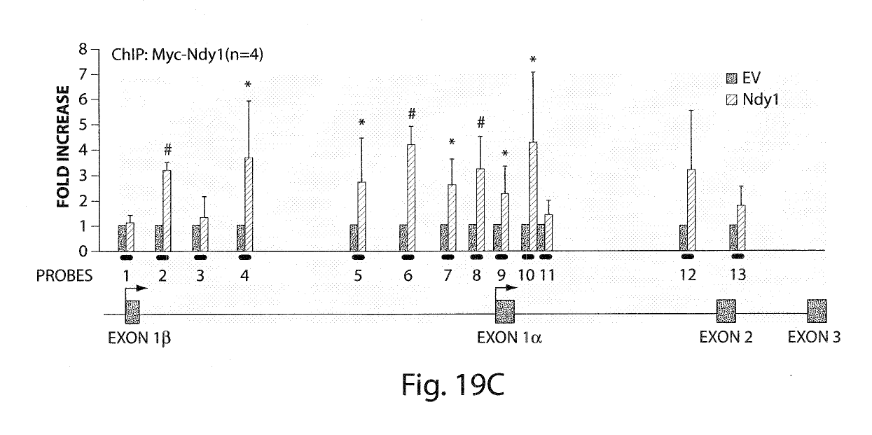

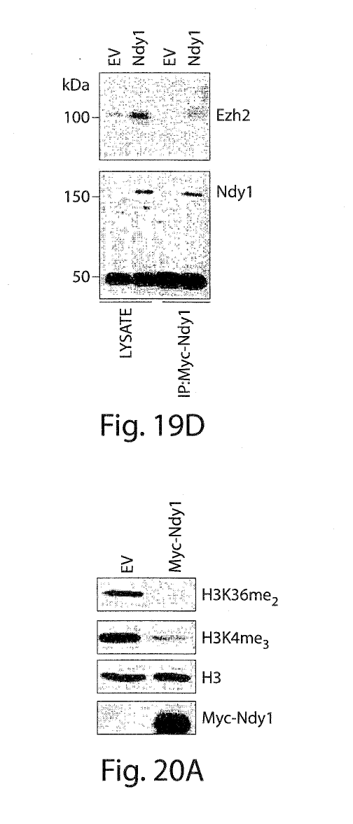

FIG. 19 panel A shows that whereas the transient knock down of each of Ndy1, Bmi1, or Ezh2 individually caused a relatively slight upregulation of p16^{Ink4a}, the simultaneous knock down of Ndy1 with either Ezh2 or Bmi1 or the simultaneous knock down of Ezh2 and Bmi1 caused a significant upregulation of p16^{Ink4a}. This surprising finding indicates that Ndy1 has additional effects that are independent of Ezh2 and Bmi1. Knocking down Bmi1 in MEFs that had been immortalized by Ndy1, caused only a partial reversion of the Ndy1 mediated repression of p16Ink4a (FIG. 19 panel B).

### Example 17

### Ndy1 Binds to and Promotes the Demethylation of Histone H3K36me2 and H3K4me3 in the Ink4a/Arf Locus

To test whether Ndy1 may regulate p16^{Ink4a} not only by upregulating Ezh2 but also by promoting histone H3 demethylation at the Ink4a/Arf locus, the Ndy1-dependent regional demethylation of histone H3 relationship to binding of Ndy1 to the Ink4a/Arf locus was tested.

ChIP analyses revealed that Myc-tagged Ndy1 binds exon1α and the transcribed regions of both the Ink4a and Arf genes (FIG. 19 panel C). Previous studies had shown that Ezh2, which is upregulated by Ndy1, also binds the Ink4a/Arf locus (Bracken, A. P., et al. (2007) Genes Dev 21, 525-30) and that Ndy1 interacts with Bmi1 and Ring1b (Sanchez, C., et al. (2007) Mol Cell Proteomics 6, 820-34; Gearhart, M. D., et al. (2006) Mol Cell Biol 26, 6880-9). To determine whether Ezh2 facilitates binding of Ndy1, interaction between Ndy1 and Ezh2 was examined. FIG. 19D shows that the two proteins indeed interact and suggests that by upregulating Ezh2, Ndy1 facilitates its own binding to the Ink4a/Arf locus.

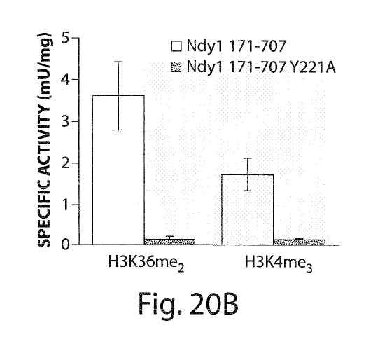

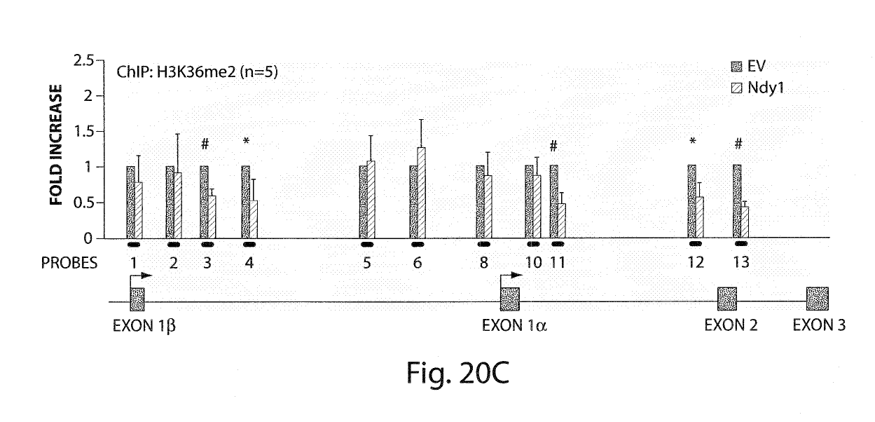

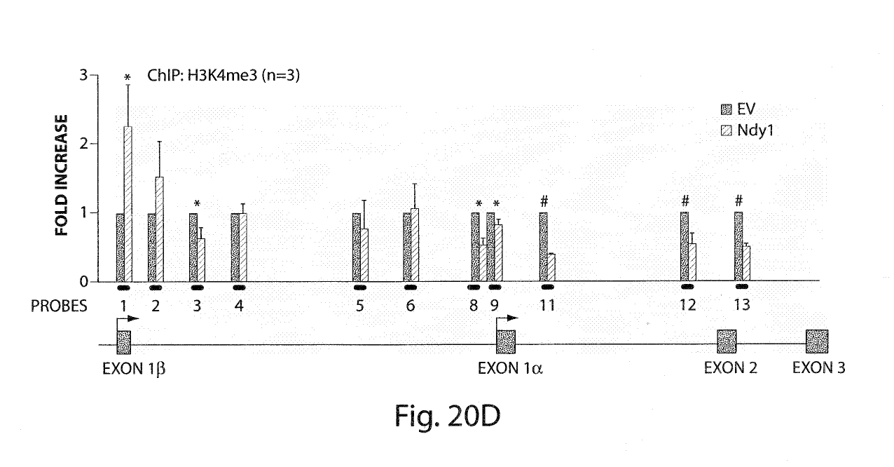

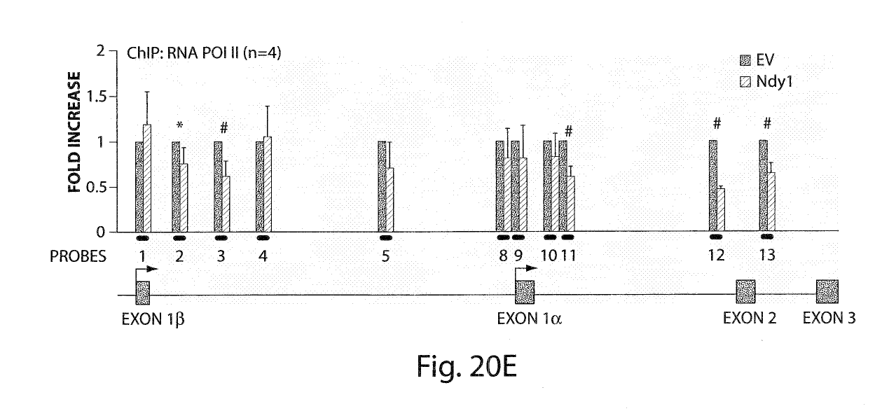

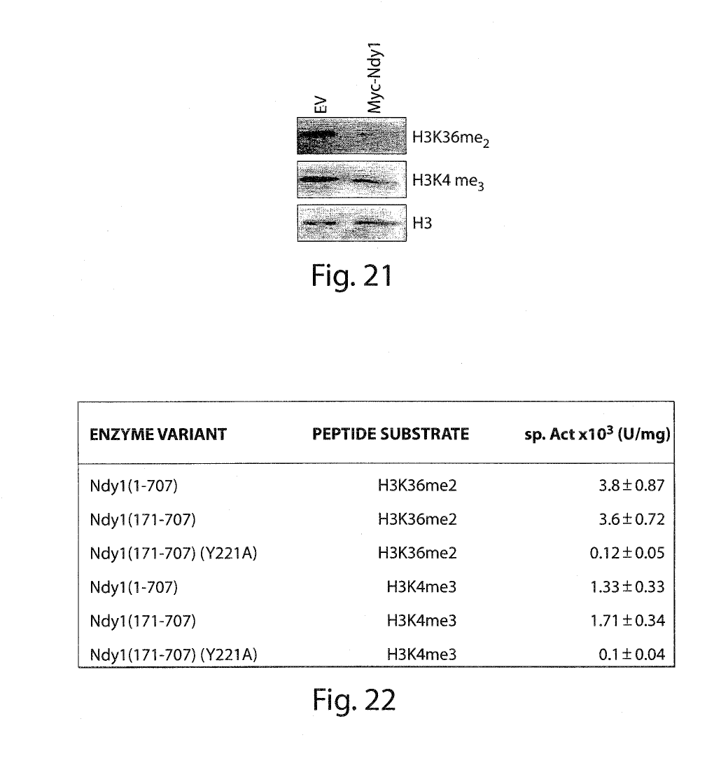

The specificity of the Ndy1 histone demethylase has been a matter of controversy (Frescas, D., et al. (2007) Nature 450, 309-13; Kim, A., et al. (2007) Mol Cell Biol 27, 1271-9). Data in examples herein show that both mouse and human Ndy1 immunoprecipitated from MEFs and IMR90 cells possesses strong H3K36me2 and weak H3K4me3 demethylase activities (FIG. 20 panel A and FIG. 21).

For assay of in vitro histone demethylation, cells from a confluent 15 cm dish were harvested and the cell pellet was resuspended in 5 packed cell volumes of Buffer A (10 mM HEPES pH 7.9, 1.5 mM MgCl₂, 10 mM KCl, 0.5 mM DTT, and 0.2 mM PMSF) and centrifuged at 1500 rpm for 5 minutes. Buffer A was added up to a final of 3 original packed cell volumes and the suspension was incubated on ice for 10 minutes. Cells were transferred to a Wheaton A Dounce homogenizer and lysed with 10 strokes. Nuclei were pelleted by centrifuging at maximum speed for 5 min in a microcentrifuge. Nuclei were resuspended in Buffer B (20 mM HEPES, pH 7.9, 100 mM KCl, 0.5 mM DTT and 0.2 mM PMSF) and disrupted by sonication. After preclearing with 20 μl protein A-agarose beads, the supernatant was incubated with either myc- or hemagglutinin-specific monoclonal antibody (5 μg/sample) and 20 μl protein A-agarose beads overnight at 4° C., the sample was washed three times with 1 ml Buffer B and the purified immune complex was incubated overnight at 4° C. with myc or HA peptide to release Ndy1/KDM2B from the beads. Demethylation reactions were carried out in 10 mM HEPES pH 7.9, 50 mM NaCl, 1 mM α-ketoglutarate, 2 mM ascorbate, 30-70 μM Fe(NH4)2(SO4)2, 0.25 mg/ml BSA and contained 6 μg of bulk histone per 90 μl reaction. Reactions were incubated at 37° C. for 1-3 hours and analyzed by 18% SDS-PAGE and Western-blotting with histone specific antibodies.

Fluorescent assays were carried out as previously described (Couture, J. F., et al. (2007) Nat Struct Mol Biol 14, 689-95) under the reaction conditions above. One unit of enzyme activity is determined as amount of Ndy1 that produces 1 μmol of formaldehyde per min, incubated with 340 μM peptide substrate at 37° C. under the above conditions. The sequences of the H3K4(me3) and H3K36(me2) peptides used in the biochemical studies were: ART-K(me3)-QTARKST (SEQ ID NO: 79) and ATGGV-K(me2)-KPHRY (SEQ ID NO: 80) respectively and were synthesized at Tufts University Core facility.

Using a fluorescence coupled demethylation assay, a bacterially expressed mouse Ndy1 fragment containing the JmjC, CXXC and PHD domains (aa 171-707) was here shown to demethylate both H3K36me2 and H3K4me3 peptides, with high and low efficiencies respectively (FIG. 20 panel B and FIG. 22). Cloning and bacterial expression of Ndy1 fragments was accomplished using fragments 1-701 and 171-701 of mouse Ndy1, which were amplified using specific primers and were cloned directly into pET102/DTOPO vector (Invitrogen) using of the TOPO-cloning methodology. The C-terminally His-tagged fusion proteins were purified from BL21(DE3) cells using Ni2+agarose beads (QIAGEN) and according to the manufacturer instructions. Flow cytometry analysis of histone modifications was determined in HEK293 cells transfected with either pIRES-GFP-empty vector (Stratagene) or the same vector containing the wild type or the H283Y JmjC domain point mutant of Ndy1. After 48 hours, cells were harvested and fixed in 1% paraformaldehyde for 10 min. Cells were washed twice with PBS, and were permeabilized with 0.5% saponin in PBS for 20 min and were blocked with 3% bovine serum albumin (BSA). Primary antibodies specific for each of total histone H3 (#9715), H3K9me2 (#9753), H3K4me3 (#9751), and H3K36me2 (#9758) were obtained from Cell Signaling and were used in a dilution of 1:400 in PBS with 3% BSA. After 1 hour cells were washed twice with PBS, and were labeled with an anti-rabbit secondary antibody in a dilution of 1:400. (Alexa Fluor 635, Invitrogen, #A31577) followed by fluorescence-activated cell sorting using a CyAn high performance flow cytometer (Dako Cytomation).

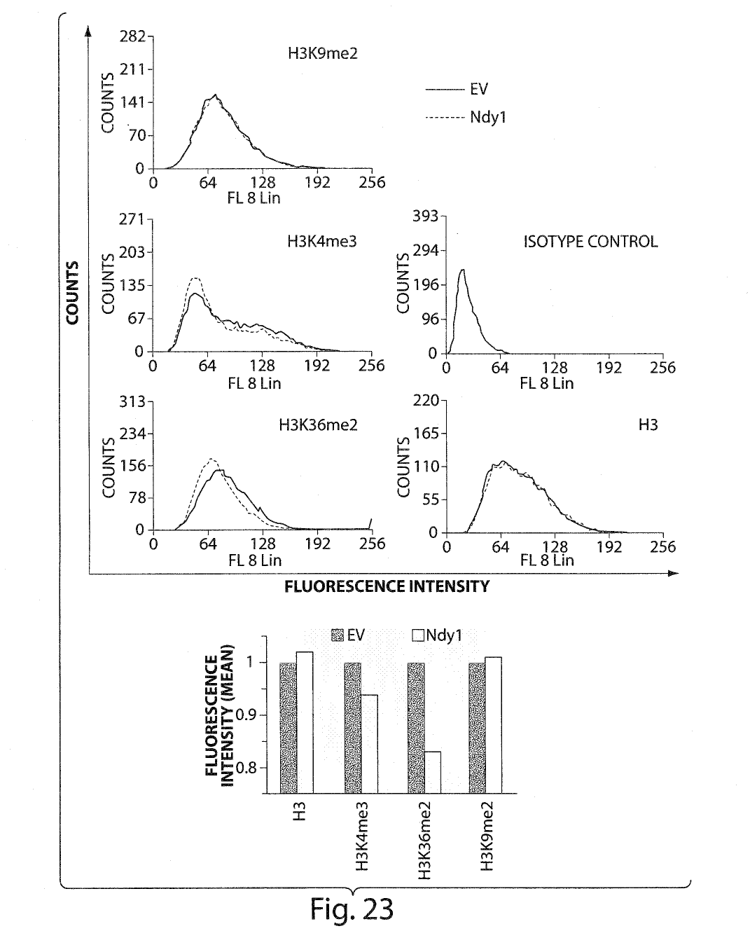

It was observed that the 171-707 Ndy1 fragment containing the Y221A mutation, which abolishes the demethylase activity of the protein (Pfau, R., et al. (2008) Proc Natl Acad Sci USA 105, 1907-12 incorporated herein in its entirety), failed to demethylate either of the peptide substrates (FIG. 20 panel B and FIG. 22). To confirm the activity and specificity of the Ndy1 histone demethylase in vivo, flow cytometry was used to quantitatively assess the global levels of histone H3K36me2 and H3K4me3 in HEK293 cells transiently transfected with Ndy1. The data obtained confirmed that Ndy1 exhibits strong histone H3K36me2 and weak histone H3K4me3 demethylase activities in vivo (FIG. 23).

ChIP assays were used to test whether the Ink4a/Arf locus-bound Ndy1 functions as a regional histone demethylase. The data obtained showed that Ndy1 overexpression down-regulates H3K36me2 (FIG. 20 panel C, probes 3-4 and 11-13) and H3K4me3 (FIG. 20 panel D, probes 3 and 11-13) in the transcribed regions of both the Ink4a and Arf genes. Ndy1 also reduced H3K4me3 in the promoter region of p16^{Ink4a} near the transcriptional start site of exon1α (FIG. 20 panel D, probes 8-9), but not in the promoter of p19^{Arf}. The inability of Ndy1 to demethylate H3K4me3 in the promoter of p19^{Arf} is related to Ndy1 repression primarily of the expression of p16^{Ink4a}.

### Example 18

### Ndy1-Driven Histone Modifications at the Ink4/Arf Locus Inhibit the Binding of RNA Polymerase II

Histone H3K36me2 and H3K4me3 correlate with active/permissive chromatin, and histone H3K27me3 is a feature of inactive/repressive chromatin (Cao, R., et al. (2002) Science 298, 1039-43; Barski, A., et al. (2007) Cell 129, 823-37; Kim, T. H., et al. (2005) Nature 436, 876-80; Kim, A., et al. (2007) Mol Cell Biol 27, 1271-9; Joshi, A. A., et al. (2005) Mol Cell 20, 971-8; Krogan, N. J., et al. (2003) Mol Cell 11, 721-9; Vermeulen, M., et al. (2007) Cell 131, 58-69; Bernstein, B. E., et al. (2005) Cell 120, 169-81; Shilatifard, A. (2006) Annu Rev Biochem 75, 243-69; Eissenberg, J. C. et al. (2006) Curr Opin Genet Dev 16, 184-90). Ndy1 demethylates H3K36me2 and H3K4me3 and enhances both trimethylation of histone H3 at K27 and the binding of Bmi1/PRC1 to the Ink4a/Arf locus. Therefore, repressing this locus was tested by inhibiting the recruitment of RNA Pol II.

ChIP analyses revealed that Ndy1 overexpression significantly downregulated the binding of RNA Pol II to the transcribed region of the Ink4a/Arf locus (FIG. 20 panel E, probes 2-3, 11-13). The demonstrated interdependence of histone H3K36me2 and H3K4me3 demethylation and RNA Pol II binding is shown by observations herein that the pattern of histone demethylation and the pattern of RNA Pol II binding are similar.

### Example 19

### Ndy1/KDM2B Protects MEFs from Ras-Induced Senescence

Overexpression of an oncogene, such as Ras, in primary fibroblasts induces premature senescence (Collado, M., et al. (2007) Cell 130, 223-33; Sharpless, N. E., et al. (2001) Nature 413, 86-91; Serrano, M., et al. (1996) Cell 85, 27-37; Brookes, S., et al. (2002) Embo J 21, 2936-45; Lin, A. W., et al. (1998) Genes Dev 12, 3008-19). To address whether Ndy1/KDM2B protects cells from oncogene-induced senescence, RasV12 was expressed in MEFs either alone or in combination with Ndy1/KDM2B or Bmi1, a known inhibitor of oncogene-induced senescence (Jacobs, J. J., et al. (1999) Nature 397, 164-8; Datta, S., et al. (2007) Cancer Res 67, 10286-95).

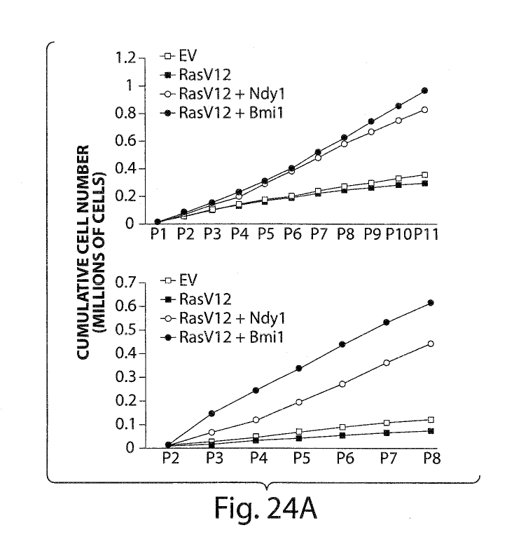

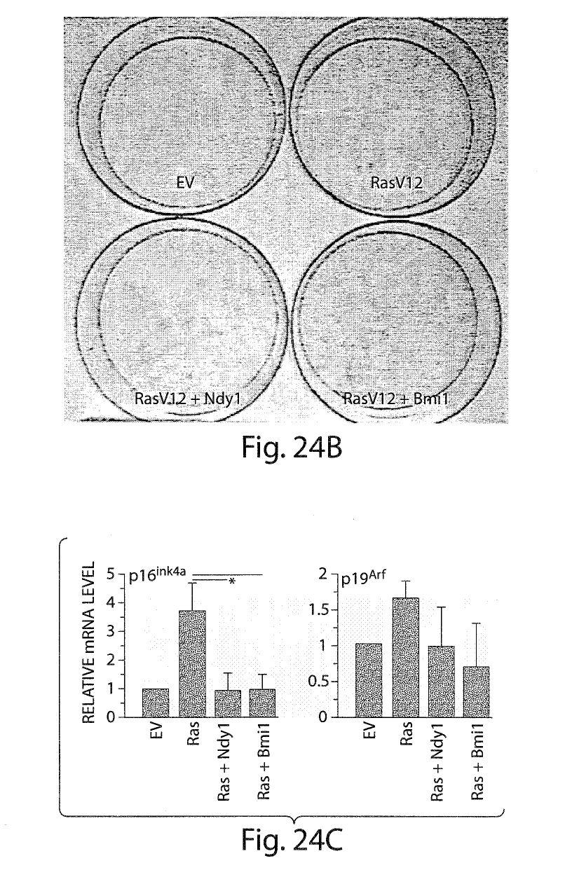

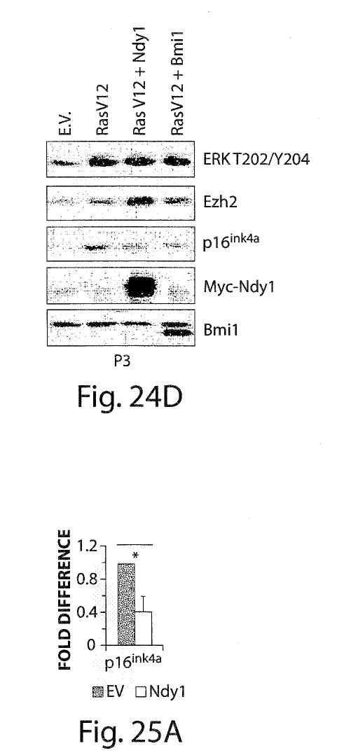

Cells were plated at two different concentrations and were counted at each passage. As shown in FIG. 24 panel A overexpression of RasV12 alone was observed to induce senescence, whereas co-expression of Ndy1/KDM2B or Bmi1 inhibited senescence. Furthermore, whereas MEFs expressing RasV12 together with Ndy1/KDM2B or Bmi1 formed colonies when plated at very low density, MEFs expressing only RasV12 did not for colonies (FIG. 24 panel B). Overall, these data show that overexpression of Ndy1/KDM2B bypasses oncogene-induced senescence and cooperates with Ras to transform MEFs. The mechanism by which Ndy1 inhibits Ras-induced senescence appears to be similar to the mechanism by which it inhibits replication induced senescence. These data show that Ndy1 also upregulates Ezh2 and inhibits the induction of p16Ink4a and p19Arf in RasV12-transduced MEFs (FIG. 24 panels C and D).

### Example 20

### Ndy1 Upregulates Ezh2 and Represses p16Ink4a in IMR90 Cells

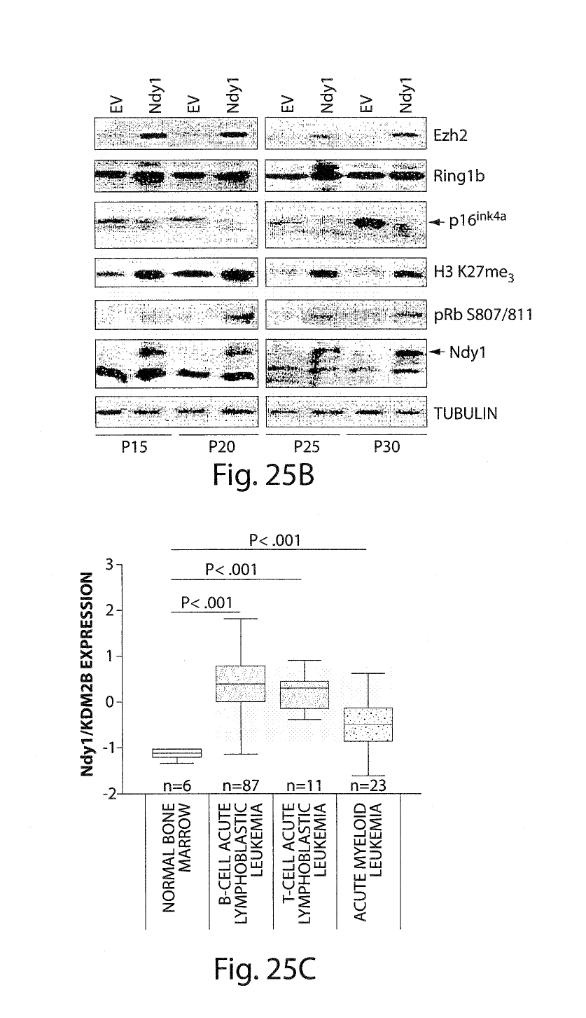

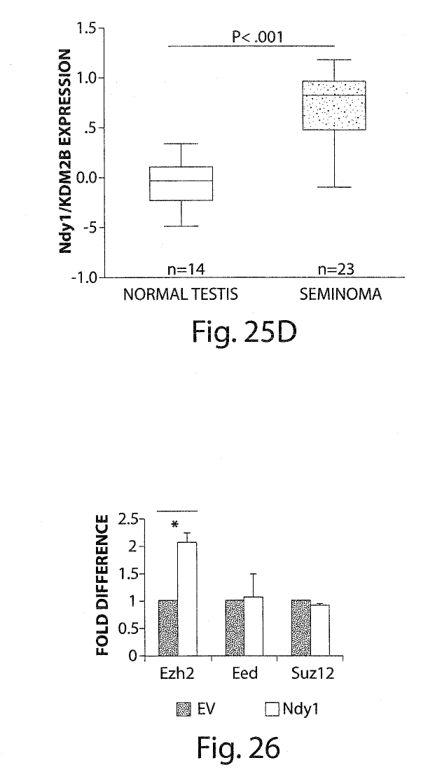

To address if Ndy1 represses the Ink4a/Arf locus in human cells, Ndy1 was overexpressed in IMR90 human fibroblasts. Data show that Ndy1 attenuated the induction of p16^{Ink4a} during passage and enhanced the phosphorylation of Rb at Ser807/811 (FIG. 25 panels A and B). In addition, Ndy1 overexpression was found in IMR90 cells to result in upregulated Ezh2 and global H3K27 tri-methylation, similar to data observed herein in MEFs (FIG. 25 panel B and FIG. 26).

### Example 21

### Ndy1 is Upregulated in Human Cancer

Activation of Ndy1 by provirus insertion in MoMuLV-induced rat T cell lymphomas and Ndy1 ability to inhibit replicative senescence strongly show that Ndy1 functions as an oncogene. These findings raised the question whether Ndy1 also functions as an oncogene in humans.

To address this question, the Oncomine online database was searched for differences in the expression of Ndy1 between normal and tumor tissues. Data mining and statistical analysis was used to measure gene expression data on Ndy1 retrieved from the Oncomine online database (http://www.oncomine.org). For statistical calculations on the expression levels of Ndy1, data from two studies (Andersson, A., et al. (2007) Leukemia 21, 1198-203; Sperger, J. M., et al. (2003) Proc Natl Acad Sci USA 100, 13350-5) were reanalyzed using the SPSS 11 statistics software. Results were expressed as mean±SEM. Differences between two groups were assessed using the two-tailed Student's t test.

The data showed that the expression of Ndy1 was significantly increased in B and T acute lymphoblastic leukemias (B-ALL and T-ALL; Andersson, A., et al. (2007) Leukemia 21, 1198-203), in acute myeloid leukemias (AML; Andersson, A., et al. (2007) Leukemia 21, 1198-203), and in seminomas (Sperger, J. M., et al. (2003) Proc Natl Acad Sci USA 100, 13350-5; FIG. 25 panels C and D, respectively). These results show that Ndy1 contributes to the development of those cancers.

### Example 22

### Ndy1 is Expressed at High Levels in Stem Cells and its Expression Declines with Differentiation

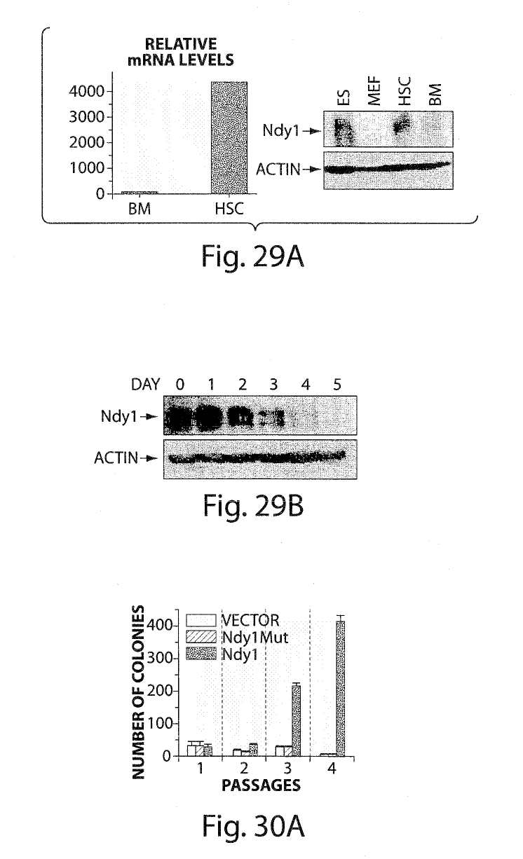

Real time RT-PCR assays comparing the expression of Ndy1 in total bone marrow and the isolated HSCs showed that Ndy1 is expressed at significantly higher levels in the HSCs (FIG. 29 panel A). Total RNA was isolated from Lin(−)Sca1⁺c-Kit⁺ cells by sorting from bone marrow cells pooled from 10 mice. Lysates of an aliquot of these cells cultured in MyeloCult media were harvested after 48 hours in culture. Western blotting of total bone marrow and short-term HSC cultures confirmed real time RT-PCR, showing that Ndy1 is expressed at significantly higher levels in the HSCs than in the total bone marrow. The Western blot showed also that the expression of Ndy1 is high in ES cells and low in MEFs (FIG. 29 panel B). Further, culturing the ES cells without a fibroblast feeder layer and without LIF (leukocyte inhibitory factor) was observed to promote differentiation into fibroblast-like cells. Differentiation was further observed to be associated with a gradual decline of Ndy1 expression. Surprisingly, overexpression of Ndy1 in ES cells interferes with differentiation showing that Ndy1 may be involved in the cycling of stem cells.

Stem cells are known to exhibit significant resistance to oxidative stress (Tothova et al. (2007) Cell 128, 325-39). Data herein show that Ndy1 inhibits oxidative stress by regulating the expression of a set of antioxidant genes (See also Polytarchou, C., et al. (2008) Mol Cell Biol 28, 7451-64, incorporated herein by reference in its entirely).

### Example 23

### Replating Efficiency of Bone Marrow Cells Infected with a MigR1.Ndy1 Retrovirus and Surface Phenotype of Ndy1-Induced Immortal Hematopoietic Cell Lines

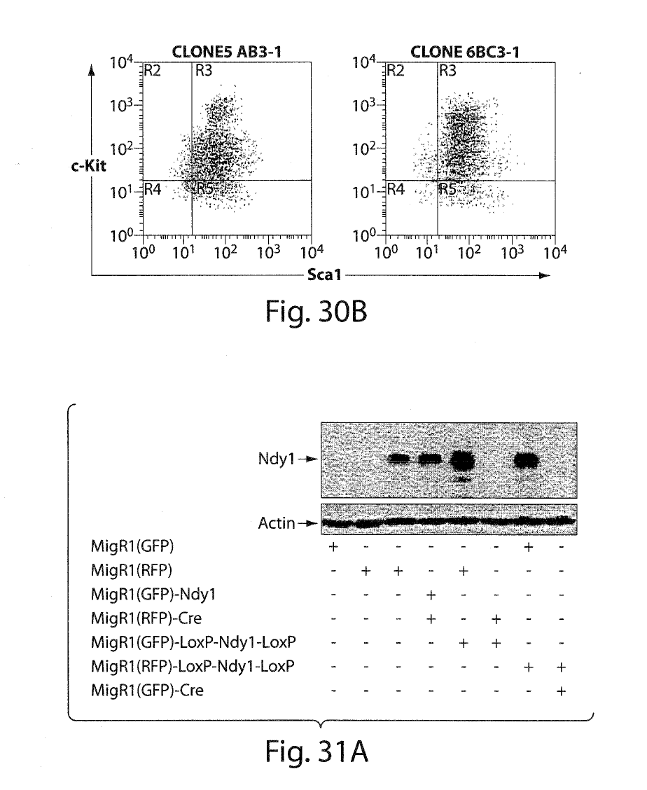

To examine replating efficiency of bone marrow cells expressing exogenous Ndy1, total bone marrow cells were infected with a MigR1.Ndy1 retrovirus or with the empty vector, and were plated in methylcellulose media containing SCF, IL-6 and IL-3. Analyzing data from replating the colonies in the same media every 10 days showed that expression of Ndy1 enhances replating efficiency. After the fourth replating, only cells infected with Ndy1 continued to grow (FIG. 30 panel A, rightmost). The colonies isolated after repeated replating were observed to grow reproducibly as continuous cell lines in media containing SCF and IL-3±IL-6. Flow cytometric analysis of phenotypes of these cells, using more than ten of these cell lines, revealed that all were Lin(−)Sca1⁺c-Kit⁺ (FIG. 30 panel B).

To determine whether Ndy1 expression indeed promotes selection of HSCs, purified HSCs were infected with the same strain of MigR1.Ndy1 retrovirus. Infected cells were grown in liquid cultures containing SCF, IL-3 and IL-6. The cell lines isolated from these analyses were observed to be identical to those isolated by replating the whole bone marrow in semisolid media.

### Example 24

### Deletion of the Exogenous Ndy1 Gene in Ndy1-Immortalized HSC-Like Cell Lines Alters the Morphology of the Cells, Inhibits Proliferation and Permits their Limited Differentiation in Culture

To determine whether the cell lines isolated by infecting HSCs with Ndy1 are indeed HSCs, an analysis was performed to determine whether the cells retain ability differentiate in culture. Data showed that the cells retained limited differentiation potential. To carry out these analyses a system was established that allows extinguishing of expression of exogenous Ndy1 gene after selection of HSC-like cells. To this end, a MigR1-based retrovirus that contains LoxP sites flanking the Ndy1 gene was generated. Two versions of this vector were generated, one carrying the gene encoding green fluorescent protein (GFP) and the other carrying the gene encoding red fluorescent protein (RFP). Cre was cloned in both MigR1(GFP) and MigR1(RFP).

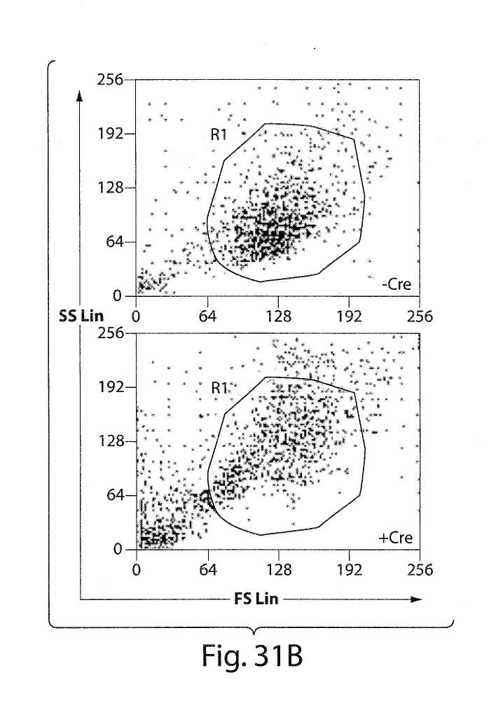

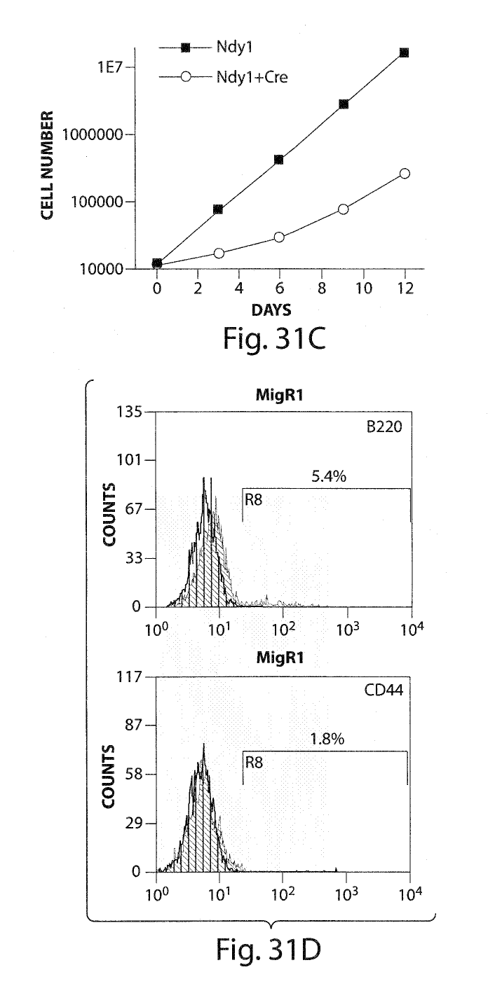

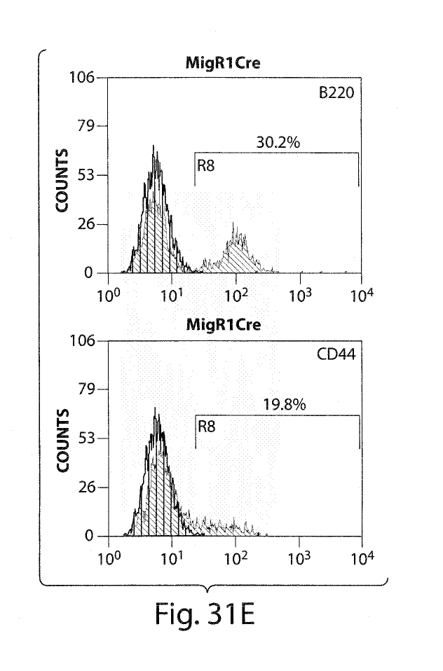

It was observed that superinfection of cells infected with the MigR1(GFP).LoxP.Ndy1.LoxP and MigR1(RFP).LoxP.Ndy1.LoxP viruses with MigR1(RFP).Cre or MigR1(GFP).Cre respectively, extinguished efficiently expression of the exogenous Ndy1 gene (FIG. 31 panel A). Following deletion of the exogenous Ndy1 gene the cells became larger and more granular, as determined by forward and side scatter analysis (FIG. 31 panel B). The increasing growth of the cells after repeated passaging (FIG. 31 panel C) was observed to be due to selection of cells not infected with the Cre virus (<1% of the original culture), which having a growth advantage were selected during passaging.

The cells superinfected with the Cre virus were plated also in methylcellulose media containing SCF, IL-6 and IL-3. Flowcytometric analysis of these cells revealed that some of them express CD34 and FcγRII. Also, some of these cells express B220 and CD44, indicating that they limited differentiation occurred during culture under these conditions (FIG. 31 panels D and E).

### Example 25

### Ndy 1 Animal Models

Transgenic animals expressing inducibly wild type Ndy1 in specific tissues were generated. An advantage of expressing Ndy1 inducibly is that overexpression of Ndy1 during development may be detrimental to embryonic survival, and ability to regulate expression would overcome such a hurdle. To generate the animals, a promoterless construct of Ndy1 was knocked in into the collagen locus of mouse ES cells. This placed control of the Ndy1 gene under the collagen promoter which is widely expressed. Expression of the gene in these cells however, is blocked by a transcription termination cassette, which was placed upstream of the gene and is flanked by LoxP sites. Expression of the gene is therefore inducible by Cre.

In addition, conditional Ndy1 knockout mice were generated. The knockout constructs have were generated and were electroporated into ES cells.

### Example 26

### In Vitro Demethylation Assays

Histone demethylase activity of Ndy1/KDM2B, while having a well accepted specificity, has been a matter of controversy. While initially reported as a H3K36me2 demethylase (Tsukada, Y. et al. (2006) Nature 439, 811-16), Ndy1/KDM2B was subsequently found to demethylate H3K4me3 but not H3K36me2 (Frescas, D. et al. (2007) Nature 450, 309-13). To clarify this issue, the reaction specificity of each of the native protein immunoprecipitated from MEFs, IRM90 cells transduced with murine or human Ndy1 retroviral constructs, and recombinant bacterially expressed fragments of the protein were examined. Data showed that Ndy1 possesses both H3K36me2 and H3K4me3 demethylase activities.

Senescence is induced by several distinct molecular mechanisms: activation of the Ink4a/Arf locus; progressive shortening of telomeres; and DNA damage (Collado, M., et al. (2007) Cell 130, 223-33; Gil, J., et al. (2006) Nat Rev Mol Cell Biol 7, 667-77; Serrano, M., et al. (1996) Cell 85, 27-37). Ink4a/Arf activation and telomere shortening may be developmentally programmed in dividing cells or they may be induced in response to DNA damage. Telomere shortening plays a critical role in the induction of senescence in human but not in mouse fibroblasts (Collado, M., et al. (2007) Cell 130, 223-33; Bodnar, A. G., et al. (1998) Science 279, 349-52). Cellular senescence contributes to both organismal aging and tumor suppression. Thus, whereas aging tissues upregulate p16^{Ink4a} and p19^{Arf}, different types of human cancer, such as melanomas, harbor inactivating mutations in the Ink4a/Arf locus (Collado, M., et al. (2007) Cell 130, 223-33; Gil, J., et al. (2006) Nat Rev Mol Cell Biol 7, 667-77).

Ndy1 protects cells from both replicative and oncogene-induced senescence (Pfau, R., et al. (2008) Proc Natl Acad Sci USA 105, 1907-12 incorporated herein in its entirety; and this report). Ndy1 may inhibit senescence by regulating redox homeostasis and by protecting cells from oxidative stress (Polytarchou, C., et al. (2008) Mol Cell Biol 28, 7451-64, hereby incorporated herein by reference in its entirely). In Examples herein, the data show that Ndy1 represses the expression of the Ink4a/Arf locus whose silencing also contributes to immortalization. Examples herein also show that Ndy1 is downregulated during senescence, along with Ezh2. The concordant downregulation of Ezh2 and Ndy1 indicated that Ndy1 may regulate Ezh2. By upregulating Ezh2, Ndy1 upregulates histone H3K27 trimethylation both globally and locally within the Ink4a/Arf locus. As a result, Ndy1 promotes the binding of Bmi1, which is known to repress the Ink4a/Arf locus. However, histone H3K27 trimethylation and Bmi1 binding were not sufficient to fully explain the Ndy1 phenotype. Data herein indeed show that Ndy1 binds the Ink4a/Arf locus and demethylates the locus-associated histones H3K36me2 and H3K4me3. These histone modifications combined, inhibit the binding of RNA Pol II and contribute to the silencing of the locus. Interestingly, Ndy1 also binds Ezh2. By upregulating Ezh2, a component of PRC2 which binds the Ink4a/Arf locus, Ndy1 may promote its own binding to the locus.

The complex role of Ndy1 in the regulation of the Ink4a/Arf locus is shown in the model in FIG. 28. According to this model, the pathways by which Ndy1 and Bmi1 repress the Ink4a/Arf locus overlap only partially, with Ndy1 promoting both the binding of Bmi1 and the demethylation of both H3K36me2 and H3K4me3. The placement of Bmi1 downstream of Ndy1 is based on the observation that Ndy1 promotes the binding of Bmi1 to the Ink4a/Arf locus and that Bmi1 alone has no effect on the H3K27 tri-methylation.

One of the earliest molecular events triggered by Ndy1 in both mouse and human fibroblasts is the JmjC domain-dependent upregulation of Ezh2, which results in the upregulation of histone H3K27 tri-methylation both globally and locally within the Ink4a/Arf locus. Ndy1 knock down had the opposite effects. Moreover, Ndy1 and Ezh2 were downregulated in concert during senescence which is characterized by Ezh2 depletion, elimination of H3K27 trimethylation, displacement of Bmi1 and transcriptional activation of the Ink4A/Arf locus (Kamminga, L. M., et al. (2006) Blood 107, 2170-9; Bracken, A. P., et al. (2006) Genes Dev 20, 1123-36). These data show that the upregulation of Ezh2 by Ndy1 plays an important role in the Ndy1 immortalization phenotype. The data herein show that Ndy1 upregulates the mRNA levels of Ezh2. However, the upregulation is modest, suggesting that Ndy1 may upregulate Ezh2 primarily via post-translational mechanisms. The physical interaction between Ndy1 and Ezh2 and the JmjC domain dependence of the upregulation of Ezh2 by Ndy1, show that Ndy1 may upregulate Ezh2 by stabilizing polycomb complexes (Sanchez, C., et al. (2007) Mol Cell Proteomics 6, 820-34; Gearhart, M. D., et al. (2006) Mol Cell Biol 26, 6880-9), perhaps via demethylation. Overexpression of Ezh2 and histone H3K27 tri-methylation promote the binding of Bmi1, which results in transcriptional repression (Bracken, A. P., et al. (2007) Genes Dev 21, 525-30).

Data herein show that the upregulation of Ezh2 by Ndy1 increases H3K27 tri-methylation and Bmi1 binding at the Ink4a/Arf locus, suggesting that Ndy1 represses the Ink4a/Arf locus and inhibits senescence at least in part by regulating Bmi1 recruitment to the locus. However, the knock down of Ndy1, Ezh2 and Bmi1, singly or in combination in MEFs, and the knockdown of Bmi1 in Ndy1-immortalized MEFs, indicates that Ndy1 cooperates with both Ezh2 and Bmi1 to repress the Ink4a/Arf locus. Further Ndy1 may elicit additional events that contribute to the immortalization phenotype. Examples herein show that Ndy1 binds the Ink4a/Arf locus and demethylates histone H3K36me2 and H3K4me3. Both H3K36me2 and H3K4me3 are associated with active/permissive chromatin (Rao, B., et al. (2005) Mol Cell Biol 25, 9447-59, Li, B., et al. (2007) Genes Dev 21, 1422-30) in the transcribed region and the promoter of a gene, respectively (Barski, A., et al. (2007) Cell 129, 823-37; Kim, T. H., et al. (2005) Nature 436, 876-80; Kim, A., et al. (2007) Mol Cell Biol 27, 1271-9; Joshi, A. A., et al. (2005) Mol Cell 20, 971-8; Krogan, N. J., et al. (2003) Mol Cell 11, 721-9; Vermeulen, M., et al. (2007) Cell 131, 58-69; Bernstein, B. E., et al. (2005) Cell 120, 169-81). Mechanistically, H3K36me2 recruits histone deacetylase complexes which contribute to the restoration of normal chromatin structure in the wake of elongating RNA Pol II in the body of transcribed genes (Joshi, A. A., et al. (2005) Mol Cell 20, 971-8; Bell, O., et al. (2007) Embo J 26, 4974-84; Li, B., et al. (2007) Genes Dev 21, 1422-30; Lee, E. R., et al. (2007) Stem Cells 25, 2191-9; Keogh, M. C., et al. (2005) Cell 123, 593-605) and H3K4me3 provides a docking site for TFIID that seeds the formation of the pre-initiation complex (Vermeulen, M., et al. (2007) Cell 131, 58-69). Therefore, by eliminating those methylation marks within the Ink4a/Arf locus, Ndy1 inhibits recruitment of RNA Pol II and functions as a transcriptional repressor.

The pattern of H3K27me3 methylation and the patterns of Ndy1 and Bmi1 binding in the Ink4a/Arf locus were observed herein to be similar. These data indicate that Ndy1 may be a component of PRC2, which promotes histone H3K27 trimethylation, or PRC1, which binds chromatin by recognizing the H3K27me3 mark, or both PRC2 and PRC1. Further, Ndy1 interacts with Ezh2, and place Ndy1 in the PRC2 complex. Other studies showing that Ndy1 co-purifies with components of PRC1, such as Ring1b and Bmi1 (Sanchez, C., et al. (2007) Mol Cell Proteomics 6, 820-34; Gearhart, M. D., et al. (2006) Mol Cell Biol 26, 6880-9), place Ndy1 into the PRC1 complex. By upregulating Ezh2, Ndy1 may promote its binding to chromatin, either directly or via recognition of the H3K27me3 mark, which is induced by the upregulated Ezh2.

Ndy1 may function as an integral component of polycomb complexes regulating H3K4me3 and H3K36me2 demethylation in concert with Ezh2-mediated H3K27 tri-methylation, to fine-tune the transcription of different genes. The interdependence of H3K27 tri-methylation and H3K36me2 and H3K4me3 demethylation is shown by the passage-dependence of the outcome of these activities in cells engineered to overexpress Ndy1. Ezh2 upregulation and H3K27 tri-methylation, the earliest known consequences of Ndy1 overexpression, may promote the binding of Bmi1- and Ndy1-containing complexes. Histone modifications induced by these complexes, including trimethylation of H3K27 and demethylation of H3K36me2 and H3K4me3, may further stimulate the binding of polycomb repressive complexes. This feed forward mechanism enhances complex binding and histone modifications with each passage, as observed herein.

Examples herein show that Ndy1 enhanced the proliferation but failed to immortalize human IMR90 cells in culture (Pfau, R., et al. (2008) Proc Natl Acad Sci USA 105, 1907-12 incorporated herein in its entirety). Given that the induction of senescence in IMR90 cells but not in MEFs is due primarily to telomere shortening, these data show that Ndy1 protects primary cells from developmentally-regulated or DNA damage-induced growth inhibitory activities, but it does not protect them from telomere shortening. Additional examples herein show that Ndy1 upregulates Ezh2, represses the expression of p16^{Ink4a}, and upregulates the phosphorylation of Rb at Ser807/811 in both mouse and human fibroblasts. Despite the inability of Ndy1 to immortalize IMR90 cells in culture, the role for Ndy1 in the regulation of PRC2 and the repression of the Ink4a/Arf locus, a role that is conserved between human and mouse and consequently other mammalian species. These data, combined with the observation that Ndy1 is upregulated in several types of human tumors, shows that Ndy1 functions as an oncogene, not only in animal but also in human cancer.

The precise mechanism by which Ndy1 regulates the phosphorylation of Rb may be to regulate the activity of cyclin-dependent kinases, which phosphorylate Rb by altering the expression or postranslational modification of the components of cyclin/cdk complexes, or to repress the expression of cdk inhibitors. Alternatively, it may directly modify Rb, thus altering its ability to be phosphorylated by cdks.

Examination of the p53 pathway in Ndy1-overexpressing MEFs revealed that p53 and its target p21^{CIP1} were significantly upregulated. Given that p53 and p21^{CIP1} promote cell cycle arrest, senescence and apoptosis (Choudhury, A. R. et al. (2007) Nat Genet 39, 99-105; Chin, L. et al. (1999) Cell 97, 527-38), this finding was surprising. Further studies however revealed that although Ndy1 does not inhibit globally the DNA damage response, it selectively abrogates the pro-senescence phenotype of p21^{CIP1}. It is possible Ndy1 alters directly or indirectly the cyclin/cdk inhibitory activity of p21^{CIP1}. Alternatively, it may alter, again directly or indirectly the cdk-independent transcriptional activities of p21^{CIP1} (Perkins, N. D. (2002) Cell Cycle 1, 39-41).

The data presented in the examples herein indicate that Ndy1 is a molecule that promotes oncogenesis, and that Ndy1 is overexpressed as a result of provirus integration in retrovirus-induced lymphomas. Moreover, the Ndy protein inhibits senescence, which is a potent tumor protective process, as found by genetic animal models and by clinical studies on tumors and precancerous lesions (Collado, M. et al. (2007) Cell 130, 223-33; Blasco, M. A. (2005) Nat Rev Genet 6, 611-22; Dimri, G. P. (2005) Cancer Cell 7, 505-12; Feldser, D. M. et al. (2007) Cancer Cell 11, 461-9). Ndy1 may function as a tumor suppressor if is overexpressed in human lymphomas and mammary adenocarcinomas (Suzuki, T. et al. (2006) Embo J 25, 3422-31; Frescas, D. et al. (2007) Nature 450, 309-13). Ndy1 appears to protect the genome from mutations (Suzuki, T. et al. (2006) Embo J 25, 3422-31; Pothof, J. et al. (2003) Genes Dev 17, 443-8). In addition, it inhibits cell growth and proliferation when overexpressed in some tumor cell lines, such as HELA cells (Pothof, J. et al. (2003) Genes Dev 17, 443-8). Ndy1 is expressed at very low levels in aggressive glioblastomas (Pothof, J. et al. (2003) Genes Dev 17, 443-8).

Data in examples herein show that Ndy1 and Ndy2 function both as oncogenes and as tumor suppressor genes and that the final balance of their pro-oncogenic and anti-oncogenic activities may be context-dependent. Thus, in lymphomas and mammary adenocarcinomas which express high levels of Ndy1, the Ndy1 protein may have a tumor-promoting role, while in glioblastomas, in which aggressiveness correlates with low levels of expression, Ndy1 may function as a tumor suppressor. Most important, examples herein identify a novel function of JmjC domain-containing proteins and provide methods and compositions to link epigenetic regulation and cancer.

### Example 27

### The Overexpression of Ndy1 in Mouse Embryo Fibroblasts Correlates with the Upregulation of Ezh2 and the Downregulation of miR-101

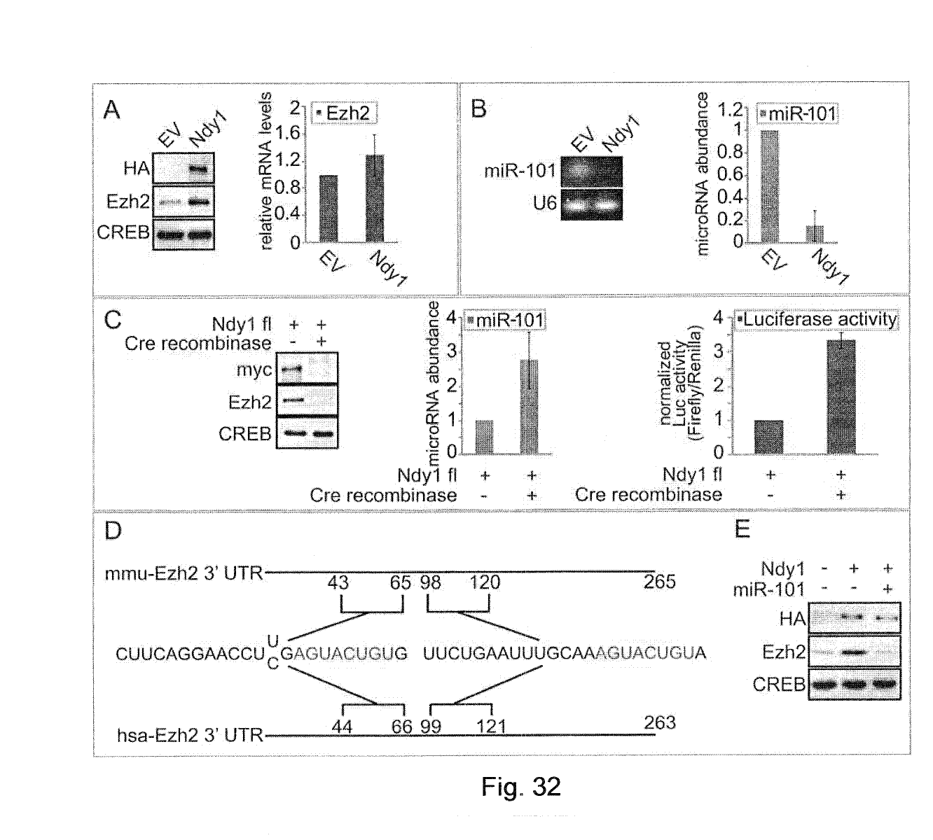

Transduction of wild type mouse embryo fibroblasts (MEFs) with MigR1-based retroviral constructs of Ndy1 promotes immortalization by bypassing replicative senescence. Examples above show that the Ndy1 immortalization phenotype is due, at least in part, to repression of the Ink4A/Arf locus and that the repression of this locus depends on the upregulation of Ezh2. By upregulating Ezh2 the Ndy1 histone H3 demethylase couples H3 demethylation at K36 with H3K27 trimethylation and represses the Ink4A/Arf locus. While Ndy1 indeed upregulates dramatically the Ezh2 protein levels, the upregulation of the Ezh2 mRNA was only modest, indicating that the upregulation of Ezh2 by Ndy1 is due to post transcriptional mechanisms (FIG. 32 panel A).

To determine if Ezh2 is regulated posttranscriptionally by miR-101, RT-PCR and quantitative real time RT-PCR were used to measure the expression of miR-101 in empty vector (EV) and pBabe-HA-Ndy1-transduced MEFs. The results in FIG. 32 panel B showed that ectopic expression of Ndy1 indeed represses the expression of miR-101. In parallel examples, MEFs transduced with a MigR1 construct of foxed myc-Ndy1 (MigR1/fl myc-Ndy1 fl/GFP) were superinfected with a retrovirus construct of the Cre recombinase or with the empty vector. Western blotting of cell lysates harvested 48 hours later revealed that both Ndy1 and Ezh2 were rapidly downregulated following the deletion of the exogenous myc-Ndy1. Real time RT-PCR using RNA isolated from the same lysates, showed that the deletion of exogenous myc-Ndy1 and the downregulation of Ezh2 were occurring simultaneous to the upregulation of miR-101 (FIG. 32 panel C, left and middle).

Genomic DNA was isolated from MEFs using DNeasy Tissue Kit (Qiagen, catalog number 69504) according to the manufacturer's instructions. The miR-101 5′-flanking region was amplified by PCR using forward primer 5′-GGGTATGTCTCAAAGGTACAGC (SEQ ID NO: 85) and reverse primer 5′-CTGCGTGTATGTCTGTGTGCT (SEQ ID NO: 86). The fragment was blunt-ligated to a SmaI-digested pGL3-basic firefly luciferase reporter gene vector (Promega, catalog number E1751), to obtain the plasmid miR-101-promoter/pGL3 which was sequence verified. For the luciferase assays, cells were transfected with the construct in combination with an RTK-Renilla construct using Lipofectamine 2000. Luciferase activity in cell lysates was determined 48 hours later using the Dual-Luciferase Reporter Assay System (Promega, catalog number TM040), according to the manufacturer's instructions. Firefly luminescence units were normalized to the respective Renilla luminescence units.

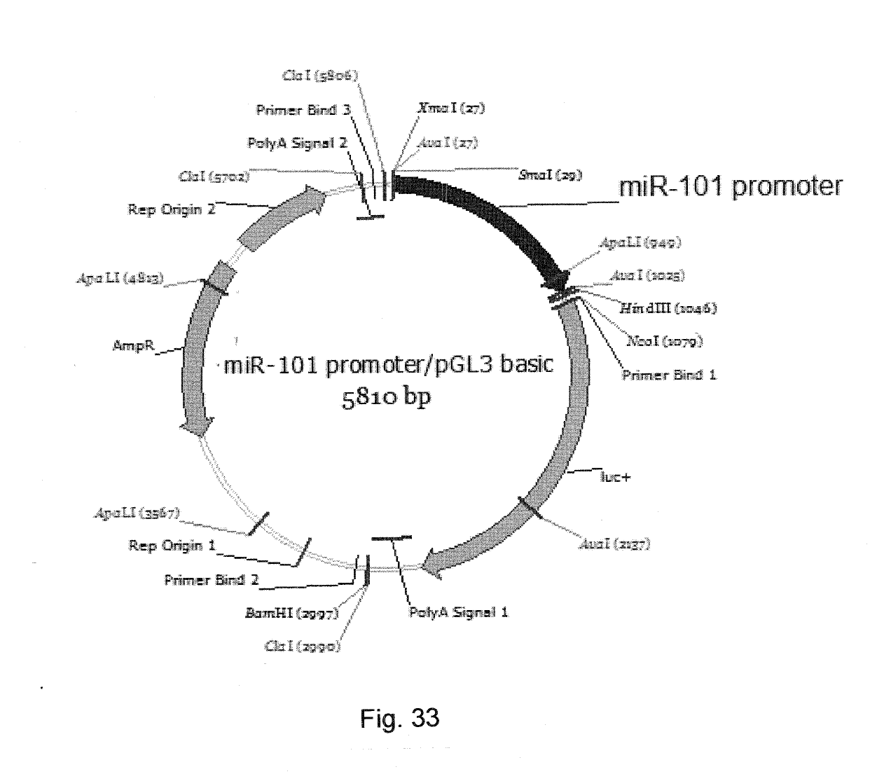

Further, the expression of a luciferase reporter under the miR-101 promoter (FIG. 33) was upregulated after the excision of the exogenous myc-Ndy1 (FIG. 32 panel C, right).

Further in support of the model that the deregulation of miR-101 is responsible for the levels of Ezh2, expression of another validated target of miR-101, Ataxin-1 was examined, in cells overexpressing Ndy1. Nuclear and cytoplasmic extracts from these cells were probed with antibodies specific for each of HA-tag (Ndy1), Ezh2, CREB and Ataxin-1, α-Tubulin, respectively. The results showed that in Ndy1 expressing cells, both these targets of miR-101 are upregulated. These data combined show that Ndy1 regulates Ezh2 by repressing miR-101.

Further, miR-101 regulations of the expression of Ezh2 were also analyzed in human cells, with human miR-101. The human and the mouse miR-101 are identical but the functional conservation between human and mouse was not previously been demonstrated (FIG. 32 panel D). Transfection of MEFs with murine miR-101, confirmed that it inhibits the expression of Ezh2 (FIG. 33). Therefore, the regulation of Ezh2 by miR-101 is conserved between humans and mice.

### Example 28

### Treatment of Mouse Embryo Fibroblasts with FGF Upregulates Ezh2, by Repressing miR-101 Via Ndy1

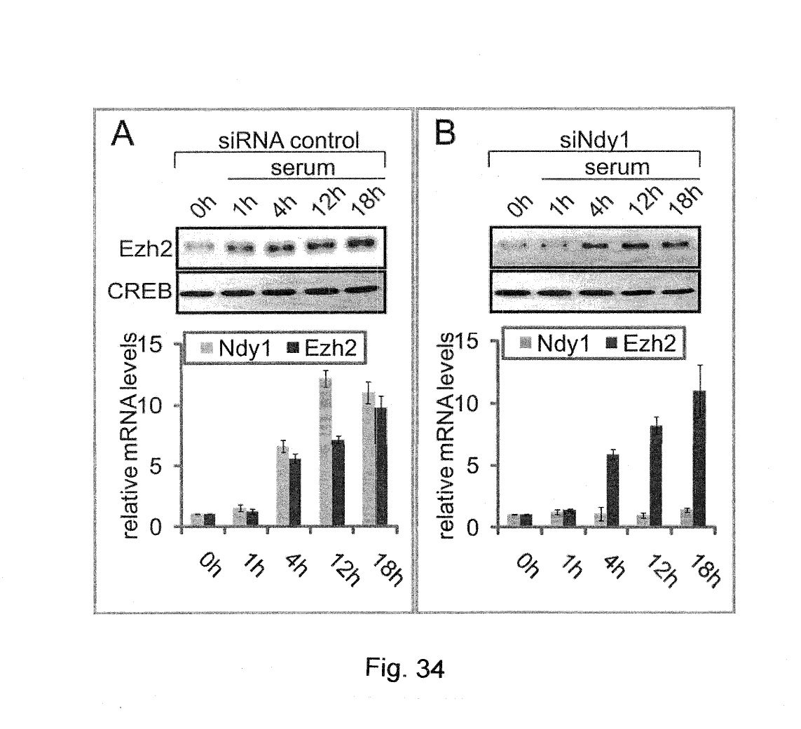

To determine whether Ndy1 is a physiological regulator of miR-101 and Ezh2 in cells growing in culture under standard conditions, expression of both were measured in cultured MEFs, in which Ndy1 was knocked-down. This result showed that the knockdown of Ndy1 down-regulates, slightly, the expression of Ezh2 but it does not affect the expression of miR-101 (FIG. 34 panels A and B). These data show that miR-101 and Ezh2 are not under the control of Ndy1 in MEFs cultured under standard conditions.

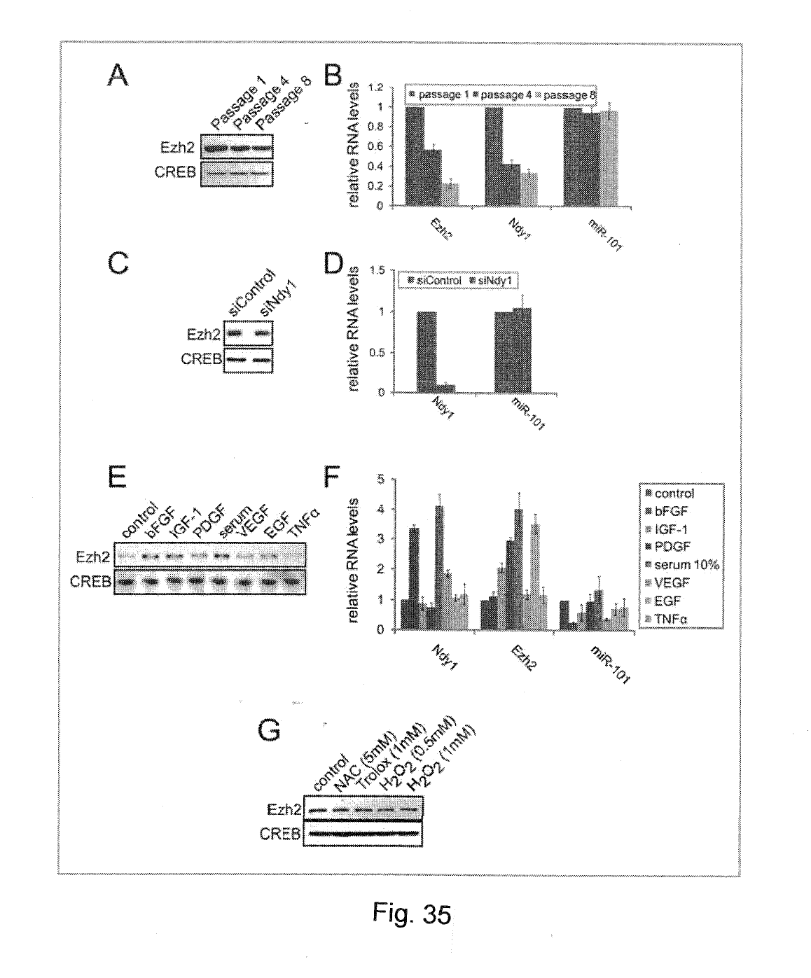

Ndy1 and Ezh2 are downregulated during passaging and senescence in MEFs. To test whether downregulation of Ezh2 and Ndy1 during senescence is causally linked via miR-101, expression of Ndy1, miR-101 and Ezh2 in passaged MEFs was analyzed. The results confirmed the downregulation of Ndy1 and Ezh2 in cells undergoing replicative senescence but they failed to show upregulation of miR-101 (FIG. 35 panels A and B). These data show that miR-101 is not responsible for the downregulation of Ezh2 during senescence.

Although Ndy1 has the ability to regulate Ezh2 via miR-101, the physiological activity of this axis was shown by data herein to be reserved for pathways other than the ones that regulate Ezh2 expression under normal culture conditions or during senescence.

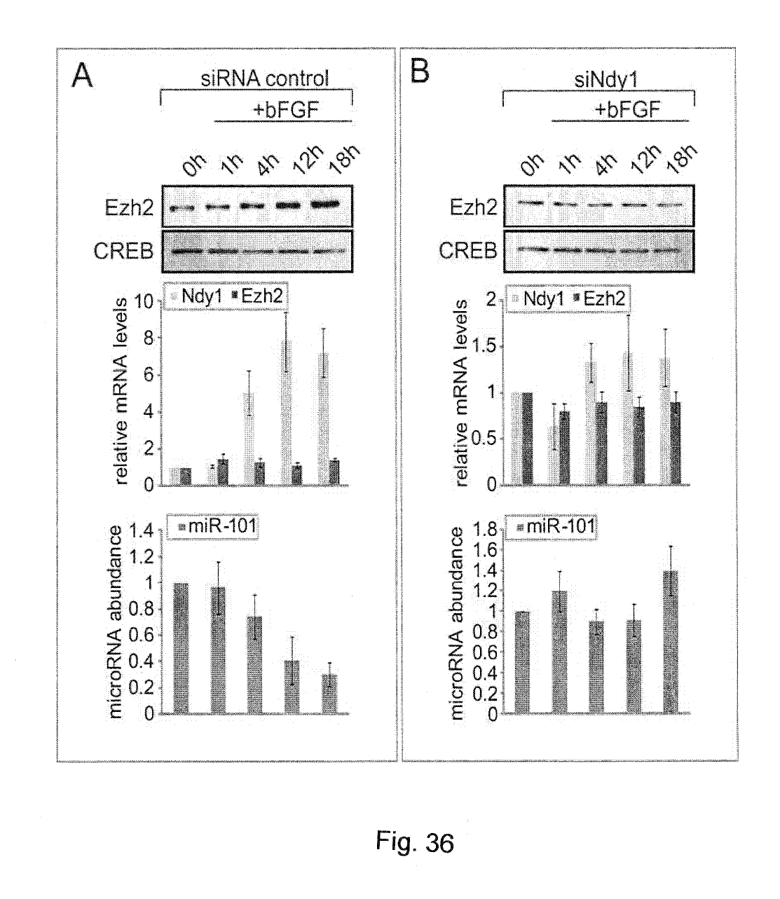

To identify a pathway regulating Ezh2 expression via the Ndy1/miR-101 axis, serum-starved MEFs were stimulated with FGF-2, IGF-1, PDGF, EGF, VEGF, TNFα or serum and were examined for expression of Ezh2, Ndy1 and miR-101 before and 24 hours after stimulation. The results showed that Ezh2 expression is significantly upregulated in MEFs stimulated with FGF-2, IGF-1, PDGF, EGF, VEGF or serum (FIG. 35 panel E). However, whereas IGF-1, PDGF, EGF and serum induced the expression of Ezh2 at both the RNA and protein levels, FGF and VEGF induced its expression primarily at the protein level, by downregulating miR-101 (FIG. 35 panel F). Moreover, FGF-2 also promoted the expression of Ndy1, whereas Ndy1 upregulation was only moderate in VEGF stimulated MEFs indicating that the upregulation of Ezh2 by FGF-2 may be due to the activation of the Ndy1/miR-101 axis. This was confirmed with analyses in which FGF-2 was used to stimulate serum-starved MEFs transfected with a control siRNA or siRNA for Ndy1 (FIG. 36). On the other hand, serum stimulation of the same cells showed that in this case, Ezh2 upregulation is not affected by Ndy1 knockdown, since serum activates transcription of the Ezh2 protein (FIG. 34). These data show that FGF-2 specifically activates the Ndy1-miR-101-Ezh2 pathway.

### Example 29

### The JmjC Domain Mutant Ndy1 H283Y, which Lacks Histone Demethylase Activity, and the Ndy1 ΔCXXC Mutant, which does not Bind DNA, do not Alter Expression of miR-101 and Ezh2

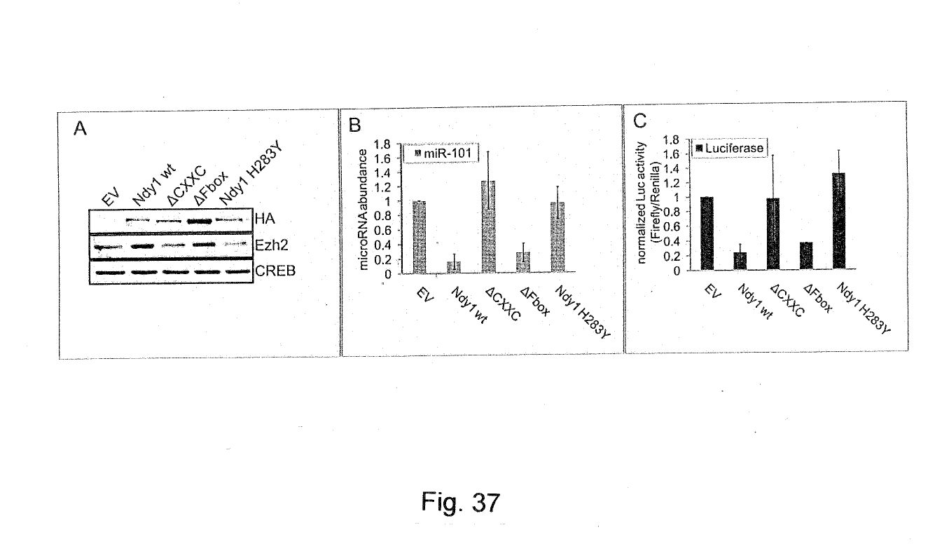

The data above raised the question of the mechanism by which Ndy1 regulates the expression of miR-101. To address this question, early passage MEFs were transduced with pBabe-based retrovirus constructs of wild type Ndy1, Ndy1 ΔCXXC, Ndy1 ΔFbox, and Ndy1 H283Y which carries a point mutation in the JmjC domain that abolishes the Ndy1 histone H3 denethylase activity. MEFs transduced with the empty pBabe vector (EV) were used as controls. Nuclear lysates of transduced cells, harvested 6 days after the transduction, were probed with antibodies specific for HA-tag (Ndy1), Ezh2 or CREB (FIG. 37 panel A). RNA isolated from the same cell was analyzed by real time RT-PCR for the abundance of the microRNA miR-101.

The results (FIG. 37 panel B) showed that whereas the wild type Ndy1 and the ΔFbox-mutant repressed the expression of miR-101, the ΔCXXC-mutant which does not bind DNA, and the Ndy1 H283Y mutant which lacks histone derethylase activity, did not repress expression. Transfection of a miR-101 promoter/luciferase reporter construct in the same cells also revealed that only the wild type Ndy1 and the ΔFbox mutant of Ndy1 repressed the activity of the promoter (FIG. 37 panel C). These data indicate that Ndy1 binds the miR-101 promoter and directly represses its activity, perhaps by promoting the modification of histones.

### Example 30

### Ndy1 and Ezh2 Binding and Histone Modifications in the miR-101 Locus in Ndy1-Overexpressing MEFs

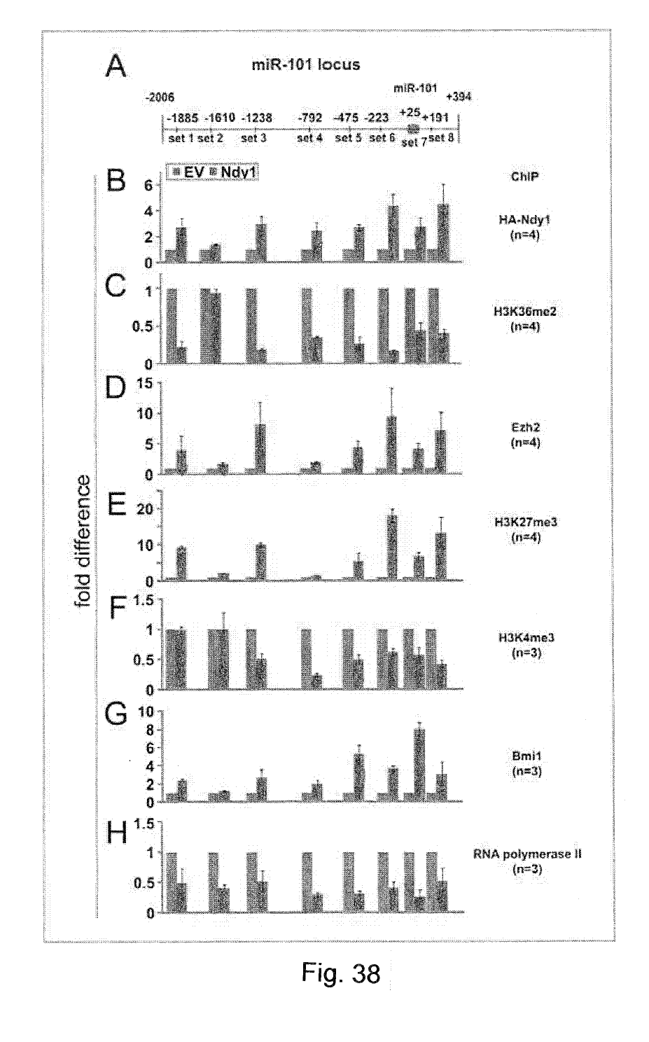

Examples above showing the repression of the Ink4A/Arf locus by Ndy1 revealed that Ndy1 binds the locus in concert with the upregulated Ezh2 and couples histone H3 demethylation at K36 with histone H3 trimethylation at K27. Whether Ndy1 and Ezh2 also bind the miR-101 locus and whether their binding is associated with changes in histone H3 methylation at K36 and K27 was therefore determined. The results (FIG. 38 panels A to H) showed that both Ndy1 and Ezh2 bind miR-101, thereby coupling histone H3K36(me2) and H3K4(me3) demethylation with histone H3 K27 trimethylation in this locus. Therefore the mechanisms of miR-101 and Ink4A/Arf repression by Ndy1 are similar. The specificity of Ndy1 binding to the miR-101 locus was confirmed by analyzing the binding of Ndy1 and Ezh2 to prdx2 locus as a negative control.

### Example 31

### Ndy1 Mediates FGF-2 Induced Proliferation and Migration, by Regulating the Levels of miR-101 and Ezh2

FGF-2 stimulation induces both proliferation and migration in various cell types. To determine the role of Ndy1 in FGF-2-driven proliferation and migration, FGF-2 was used to stimulate serum-starved MEFs which were transfected with control RNA, siRNA for Ndy1, miR-101 or with siRNA for Ndy1+miR-101.

To determine cell number, the 3-[4,5-dimethylthiazol-2-yl]-2,5-dimethyltetrazolium bromide (MTT, Invitrogen, catalog number M6494) assay was used as described in Polytarchou, C et al., 2005 J. Biol. Chem. 280: 40428-35. Cells were seeded at 2×10⁴ cells/well in 24-well tissue culture plates. Twenty-four hours later, cells were serum-starved in medium without FBS for 16 h. Culture medium was replaced with fresh medium containing FGF-2 or control carrier, and the number of cells was determined. Results were confirmed by direct counting of cells under the microscope, using a standard hemocytometer.

Migration assays were performed using 24-well microchemotaxis chambers (Costar) with uncoated polycarbonate membranes (pore size 8 μm). Briefly, cells were harvested and resuspended at 5×10⁴ cells/0.15 ml in DMEM containing 0.25% BSA. The bottom chamber of each transwell unit contained 0.6 ml of DMEM supplemented with 0.25% BSA and FGF-2 (20 ng/ml) or carrier as a control. The plates were incubated for 6 h at 37° C. and the filters were fixed with 4% paraformaldehyde in PBS and stained with 0.1% crystal violet solution. The cells that migrated through the filter were counted using a grid and an Optech microscope at 20× magnification.

To assay wound healing, cells were plated on 6-well plates, incubated in DMEM containing 10% FBS until confluence and were serum starved for 16 hours. Wounds were introduced to the confluent cell monolayer with a plastic pipette tip. Wells were washed three times to remove floating cells, and were cultured in DMEM without FBS, in the presence or absence of FGF-2 (20 ng/ml). Twelve and twenty four hours later, the wound area was photographed using a Nikon Phase Contrast-2 ELWD 0.3 Diaphot TMD inverted microscope with a 10× objective and a Spot charge-coupled-device camera (Diagnostic Instruments).

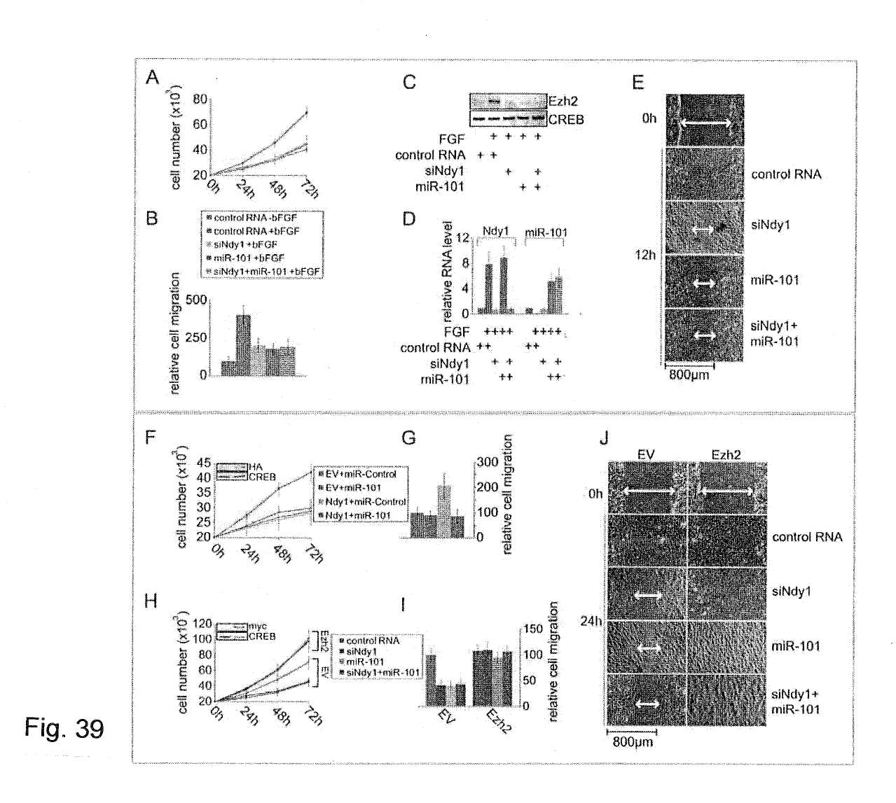

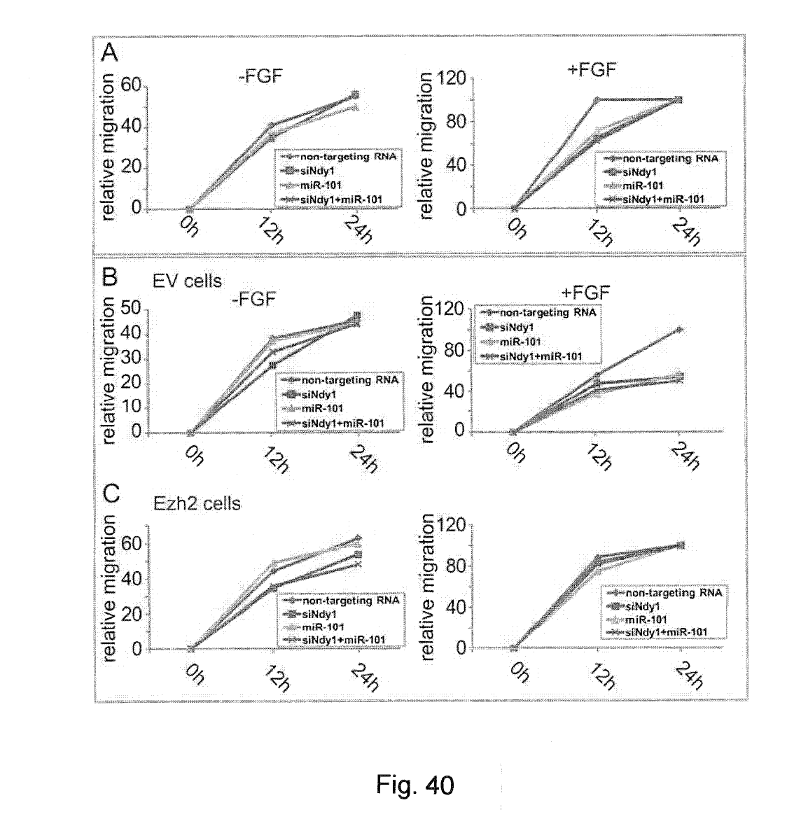

FGF-2 was observed to increase the proliferation rate and directional and random migration of MEFs transfected with control RNA (FIG. 39 panels A, B, and E, and FIG. 40 panel A). Further, this effect was observed to be greatly limited as a result of knocking down Ndy1, introducing miR-101 or both. Nuclear extracts of these cells were probed with antibodies for Ezh2 and CREB, at the 48 hour-time point. RNA isolated from the same cell was analyzed by real time RT-PCR for abundance of microRNA miR-101 and Ndy1 mRNA. The results (FIG. 39 panels C and D) showed that introducing miR-101 or siRNA for Ndy1 in MEFs was sufficient to block upregulation by FGF-2-induced Ezh2. These data show that FGF-2 strongly induced proliferation and migration in an Ndy1-dependent manner.

Further, overexpression of Ndy1 increased proliferation and directional migration (FIG. 39 panels F and G) in serum-starved MEFs and these effects were abrogated by introducing miR-101 in the cells. These data show that Ndy1 overexpression desensitizes MEFs to FGF-2.

It is here envisioned that the Ndy1-miR-101-Ezh2 pathway is at least partly responsible for FGF-2 driven proliferation and migration, and that overexpression of Ezh2 in MEFs renders the cells insensitive to Ndy1 or miR-101 alterations. Therefore early passage MEFs were transduced with pBabe-based retrovirus constructs of myc-Ezh2. MEFs transduced with the empty pBabe vector (EV) were used as controls. These cells were transfected with control RNA, siRNA for Ndy1, miR-101 or siRNA for Ndy1+miR-101, and were serum starved and stimulated with FGF-2. The growth curves of these cells show that knocking down Ndy1 or introducing miR-101 in cells that already have high levels of Ezh2, has no effect on FGF-2 driven proliferation (FIG. 39 panel H). Also, directional and random FGF-2 driven cell motility remained unaffected by Ndy1 or miR-101 alterations in Ezh2 overexpressing cells (FIG. 39 panels I and J; FIG. 40 panels B and C).

### Example 32

### Ndy1 Knockdown Decreases FGF-2-Induced Tube Formation of Human Umbilical Cord Vein Endothelial Cells, an Effect that is Reversed by Ezh2 Overexpression

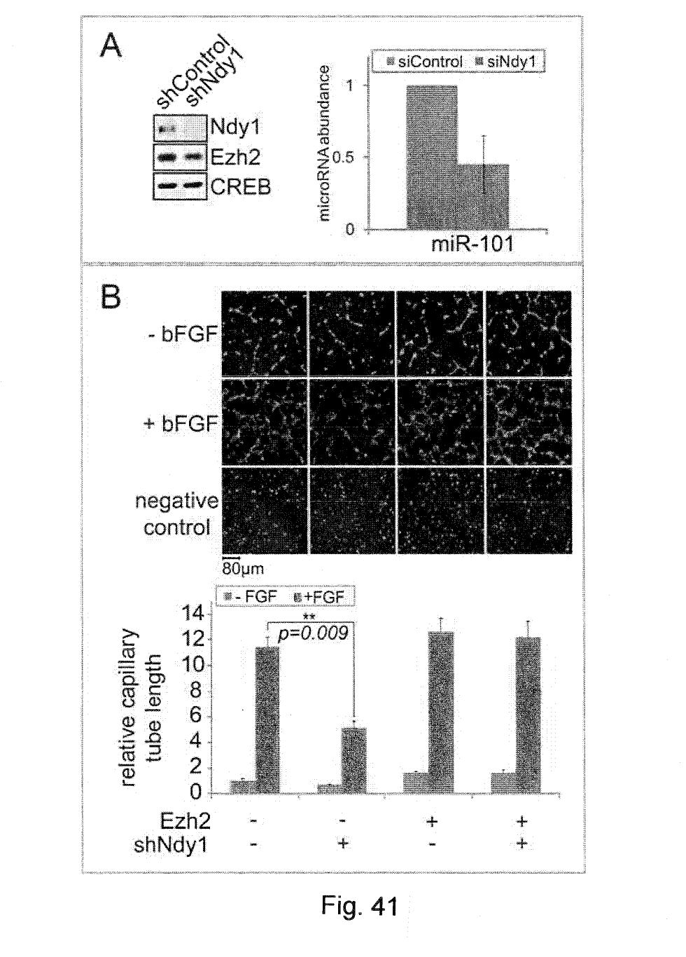

The results obtained above raised the question of the importance of the role of Ndy1 in other FGF-2-induced functions. FGF-2 is a major angiogenic factor and in this capacity is considered to be stronger even than VEGF. Since tube formation requires both proliferation and migration, and since both of these functions are Ndy1-mediated in FGF-2 stimulated cells, the effect of Ndy1 knockdown in the angiogenic properties of Human Umbilical cord Vein Endothelial Cells was examined. To address this question, HUVECs were transduced with a lentiviral-based shRNA for Ndy1. Cells transduced with an shRNA specific for GFP were used as control (shControl). Nuclear lysates from these cells, harvested two days after the end of the selection, were probed with antibodies specific for each of Ndy1, Ezh2 and CREB. RNA isolated from the same cells was analyzed by real time RT-PCR for abundance of microRNA miR-101. The results (FIG. 41 panel A) showed that knockdown of Ndy1 downregulates the expression of Ezh2 and upregulates expression of miR-101. These findings showed that miR-101 and Ezh2 are under the control of Ndy1 in HUVECs cultured under standard conditions.

HUVECs were then transduced with a pBabe-based retrovirus construct of myc-Ezh2. Cells transduced with the empty pBabe vector (EV) were used as a negative control. These cells were subsequently transduced with a lentiviral-based shRNA for Ndy1 (shNdy1) or GFP (shControl). After the second selection, the engineered HUVECs were seeded on Cultrex-RGF-BME in the presence or absence of FGF-2.

The in vitro tube formation assay was performed using the In Vitro Angiogenesis Assay Kit (Trevigen, catalog number 3470-096-K). Briefly, Cultrex-Reduced-Growth-Factor-Basement-Membrane-Extract (Cultrex-RGF-BME) was used to coat the wells of 96-well tissue culture plates (0.04 ml/well) and left to solidify for 1 h at 37° C.; 20,000 HUVECs were then suspended in 0.15 ml of Endothelial cell basal medium (PromoCell # C-222210) and added to each well, with either FGF-2 (20 ng/ml) or carrier. After 6 h of incubation at 37° C., the medium was removed, the cells were fixed and stained with Calcein AM, and the total length of capillary tubes was measured in the total area of the wells using image analysis software (Angioquant). Photographs were processed using identical settings for capturing and further processing.

Ndy1 knockdown was observed to be sufficient to reverse the FGF-2-induced increase in the total length of the capillary tubes formed (FIG. 41 panel B) in cells infected with the empty vector. In cells that overexpressed Ezh2, Ndy1 knockdown had no effect on the FGF-2 induced tube formation (FIG. 41 panel B).

### Example 33

### MiR-101 and Ezh2 Controlled by Ndy1 in Tumor Cell Lines Expressing High Levels of FGF-2

FGF signaling extends to many physiological roles in the adult organism, including the regulation of angiogenesis and wound repair. Further, deregulation of FGF signaling can promote tumor development by directly driving cancer cell proliferation and survival, and by supporting tumor angiogenesis. Since it is herein envisioned that Ndy1 is mediating FGF-2 biological functions, it was important to analyze these mechanisms in oncogenesis in vivo. Accordingly, 25 well-known cancer cell lines were obtained and examined with real time RT-PCR to determine amounts of FGF-2 mRNA.

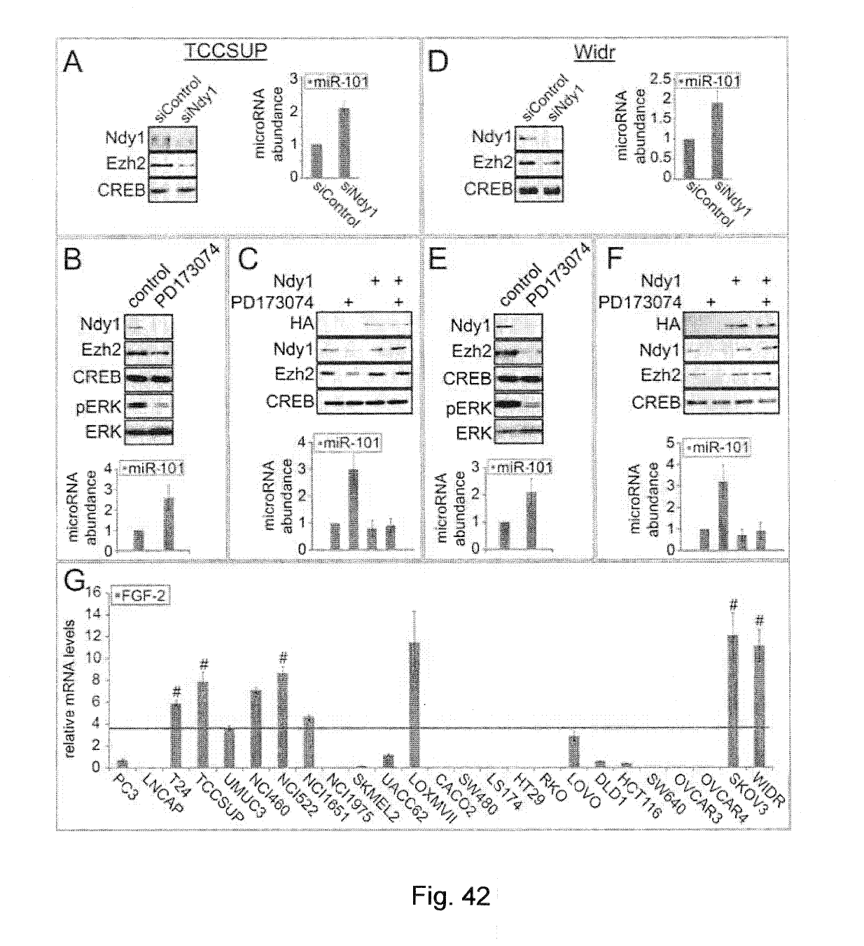

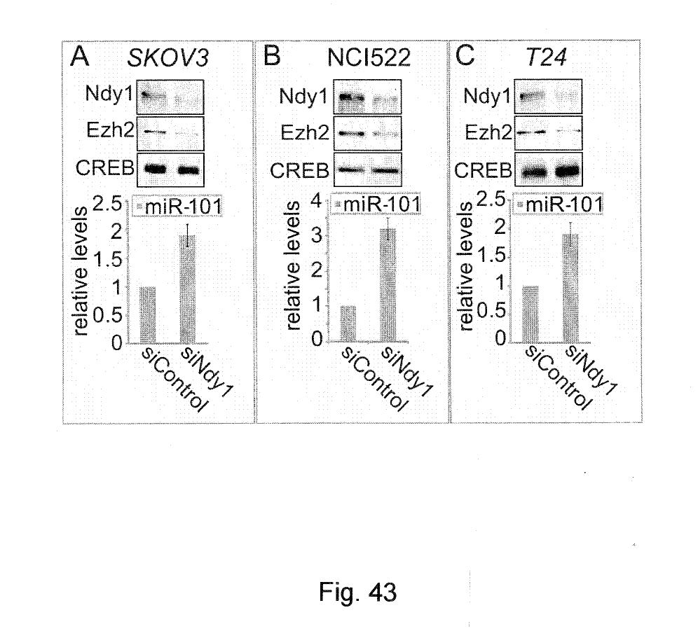

Of these cell lines, eight were observed to have expressed higher levels of FGF-2 than others of these cell lines. These eight high FGF-2 expressing cell lines were then transfected with siRNA for Ndy1 or a control siRNA. Nuclear extracts of these cells, collected 48 hours after the transfection, were probed with antibodies specific for each of Ndy1, Ezh2 and CREB. RNA isolated from these cells was analyzed by real time RT-PCR for abundance of microRNA miR-101. The results (FIG. 42 panels A and D, FIG. 43) showed that Ndy1 knockdown in five cell lines (TCCSUP, WIDR, SKOV3, T24, NCI522) of these eight cell lines downregulated Ezh2, by upregulating expression of miR-101. The five cell lines were obtained from patients having each of transitional cell carcinoma, lung cancer, colon cancer, ovarian cancer, and bladder cancer.

As a next step, FGF signaling was examined to determine importance for Ezh2 upregulation by Ndy1 in these cells. To address this issue, TCCSUP or WIDR cells, two of the above five cell lines, were treated with PD173074, an inhibitor of both FGFR1 and both FGFR3. Under these conditions the FGF signaling was blocked, and it was observed in these blocked cells that Ndy1 and Ezh2 protein levels were downregulated, and at the same time miR-101 levels were upregulated as shown in FIG. 42 panels B and E. The level of ERK1/2 phosphorylation was used to measure the effect of PD173074 on FGF signaling.

To examine if ectopic expression of Ndy1 reverses the effects of PD173074, namely miR-101 upregulation and Ezh2 downregulation, TCCSUP or WIDR cells were transduced with a pBabe-based retroviral Ndy1 overexpression construct. Cells transduced with the empty pBabe vector were used as control. The engineered cells were subsequently treated with PD173074. Nuclear lysates from these cells were probed with antibodies specific for each of HA-tag (exogenous Ndy1), Ndy1 (both exogenous and endogenous), Ezh2, and CREB. RNA isolated from the same cells was used to measure miR-101 levels with real time RT-PCR. The results (FIG. 42 panels C and F) show that when Ndy1 is overexpressed, treatment with PD173074 has no effect on the levels of Ezh2 or miR-101, rendering the pathway immune to the effects of FGF signaling.

### Example 34

### Correlation of Ndy1, miR-101 and Ezh2 Expression in MoMuLV-Induced Rat Lymphomas and Primary Human Tumors

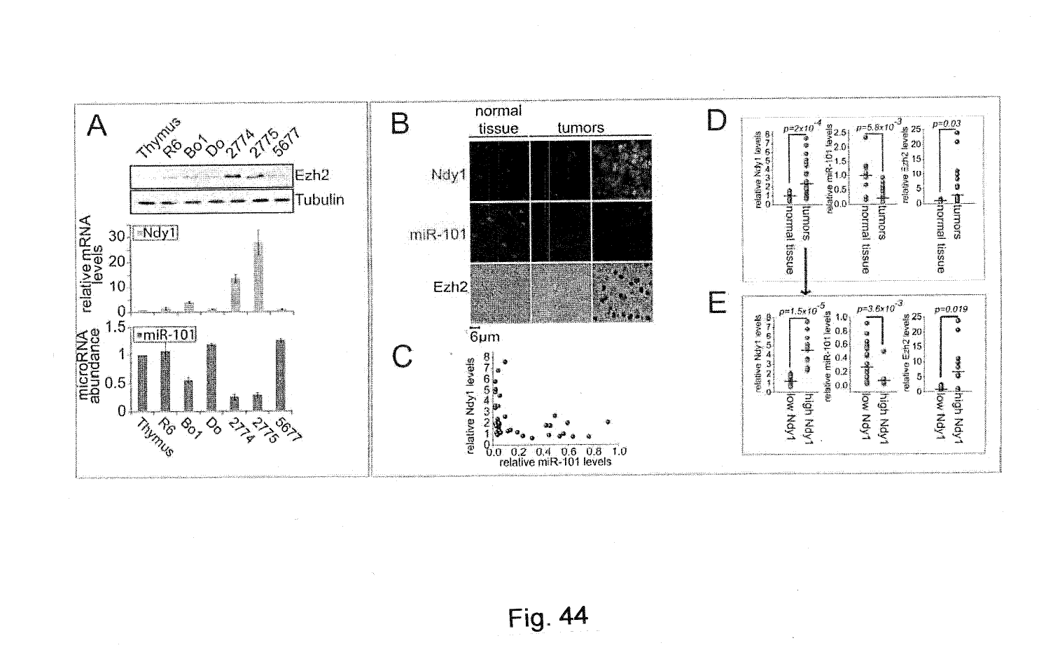

Ndy1 was first discovered as a common integration site of Moloney Murine Leukemia virus in rat lymphomas. These tumors were used to examine the correlation of Ndy1 overexpression with miR-101 upregulation and Ezh2 expression. Total cell lysates from these tumors were analyzed by western blot with antibodies specific for each of Ezh2 and α-Tubulin. RNA isolated by the same samples was analyzed with real time RT-PCR for the expression of Ndy1 and miR-101. The results showed, clearly, that Ndy1 overexpression (FIG. 44 panel A, middle) affects the levels of Ezh2 (FIG. 44 panel A, upper) by downregulating the expression of miR-101 (FIG. 44 panel A, lower).

To investigate if Ndy1 deregulation is a common feature in human malignancies, expression in tumors was analyzed. As it can be hypothesized that miR-101 is downregulated and this dowregulation is correlated with Ezh2 overexpression in bladder carcinomas, therefore, a bladder-specific tissue microarray, containing 40 bladder carcinomas and eight normal urocystic tissues for Ndy1, Ezh2 and miR-101 expression was analyzed, by in situ hybridization (Ndy1, miR-101) and immunohistochemistry (Ezh2) (FIG. 44 panel B).

For in situ hybridization, Mircury LNA Detection probe 5′-end labeled with DIG for mmu-miR-101 (Exiqon, catalog number 38475-01) or 56-FAM labelled for Ndy1 (Exiqon, TTCCCAGTCCATCCTTTTCTCG; SEQ ID NO: 81) were used as previously described with modifications. Thin 5 μm sections of lung cancer tumors or paraffin embedded bladder carcinoma microarrays (US Biomax, catalog number BL481) were deparaffinized in xylene, 2×40 mm on a 50 rpm shaker, followed by 5 min each in serial dilution of ethanol (100%, 100%, 75%, 50% and 25%) and followed by 2 changes of DEPC-ddH₂O, Slides were then submerged for 5 min in 0.2 N HCl, washed with DEPC-PBS, digested with proteinase K (40 μg/ml) for 45 min at 25° C., rinsed in 0.2% glycine/DEPC-PBS, 3×DEPC-PBS and postfixed with 4% formaldehyde in PBS for 10 min. Slides were then rinsed twice with DEPC-PBS, treated with acetylation buffer (300 μl acetic anhydride, 670 μl triethanolamine, 250 μl of 12 N HCl per 48 ml ddH₂O) and then rinsed 4 times in DEPC-PBS followed by 2 rinses in 5×SSC. Slides for detection of mmu-miR-101 were pre-hybridized at 46° C. and for Ndy1 at 52° C. for 2 hrs in hybridization buffer (50% formamide, 5×SSC, 0.1% Tween-20, adjusted to pH 6.0 with 9.2 mM citric acid, 50 μg/ml heparin, 500 μg/ml yeast tRNA) in a humidified chamber (50% formamide, 5×SSC). Following pre-hybridization, slides were hybridized overnight at 46° C. or 52° C., respectively, in a humidified chamber, using 20 nM of probe in pre-warmed hybridization buffer. Sections were rinsed twice in 5×SSC, followed by 3 washes of 20 min at 46° C. or 52° C. in 50% formamide/2×SSC. For Ndy1 detection, sections were then rinsed 5 times in PBS/0.1% Tween-20 (PBST) and coverslipped in Vectashield mounting medium with Dapi (Vector Labs). For mmu-miR-101 detection, sections were rinsed five times in PBS-0.1% Tween-20 (PBST), and blocked for 1 hour in blocking solution (2% sheep serum, BSA 2 mg/ml in PBST). Antibody specific for DIG-AP Fab fragments (Roche Applied Science, catalog number 11093274910) was applied to sections which were incubated overnight at 4° C. Next, slides were washed two times in PBST for 10 min each and washed three times for 10 min each in 0.1 M Tris-HCl (pH 7.5)/0.15 M NaCl, followed by equilibration with 1M Tris (pH 8.2) for 10 min and the Fast Red (Roche Applied Science, catalog number 11496549001) solution [1 tablet per 2 ml of 0.1 M Tris-HCl (pH 8.2)]. After a 30 min incubation in the dark, slides were washed three times in PBST for 10 min and coverslipped in Vectashield mounting medium with 4,6′-diamidino-2-phenylindole (DAPI; Vector Labs, catalog number H-1200). The same procedure was used for in situ hybridization for Ndy1 in 293 T cells, with minor modifications. Cells were plated on culture slides (BD Falcon, catalog number REF 354108) with full DMEM, grown to confluence, and were cross-linked with 4% paraformaldehyde in PBS and the procedure was then carried out as above. Images were obtained using a Nikon Eclipse 80i microscope and a Spot charge-coupled device camera (Diagnostic Instruments). Photographs were processed using identical settings for capturing and further processing.

Sections of tumors were deparaffinized in xylene, 3×5 min, followed by 10 min each in serial dilution of ethanol (100%, 100%, 95% and 95%) and followed by 2 changes of ddH₂O. Antigen unmasking was achieved by boiling the slides (95-99° C.) for 10 min, in 10 mM sodium citrate buffer pH 6.0. Sections were then rinsed 3 times in ddH₂O, incubated in 3% H₂O₂ for 10 minutes, washed twice in ddH₂O, 1 time in TBS/0.1% Tween-20 (TBST) and blocked for 1 hr in blocking solution (5% normal goat serum, TBST). Ezh2 antibody was diluted in blocking solution (1:50) and applied on sections overnight at 4° C. Next, slides were washed 3 times in TBST for 5 min each, incubated with anti-rabbit HRP diluted in blocking solution (1:500), respectively for 1 hr at room temperature. Slides were washed 3 times, in TBST for 5 min each, stained with the DAB substrate kit for peroxidase (Vector Laboratories, catalog number SK-4100), dehydrated and mounted in Cytoseal XYL (Richard-Allan Scientific, catalog number 8312-4).

The results showed that the tumors expressed higher levels of Ndy1 and Ezh2, and lower levels of miR-101 than normal tissue (FIG. 44 panel C). This correlation was found also in tumors that expressed higher levels of Ndy1, and in those that express lower levels (FIG. 44 panel D). A significant non-linear inverse correlation was thereby established between Ndy1 and miR-101 (FIG. 44 panel E, Pearson's correlation coefficient: −0.37, significance: 0.018).

Overall, these results show that Ndy1 overexpression is a common feature in bladder cancer, and that this protein correlates well with miR-101 downregulation and Ezh2 overexpression, which has thereby been shown to be a marker of cancer aggressiveness, invasiveness and poor prognosis for the patient. These data indicate that levels of one or more of proteins Ndy1 and Ezh2, and of miR-101 are useful for diagnosis and prognosis of cancer and susceptibility to cancer.

Histone demethylases and other chromatin modifying enzymes have been linked primarily to stem cell renewal and differentiation, and a subset of these play a major role in oncogenesis. However, very little is known about the integration of these enzymes into the signaling machinery. Examples herein show links of FGF-2 and the histone H3 demethylase Ndy1/KDM2B to the histone H3K27 methyltransferase Ezh2 via the microRNA miR-101 in both normal and cancer cells. Specifically, FGF-2, a growth factor that plays major role in stem cell renewal, cell proliferation, cell migration and angiogensis, is here shown to induce expression of histone H3 demethylase Ndy1/KDM2B, which in turn represses miR-101, a post-transcriptional regulator of Ezh2. Repression of miR-101 by the FGF-2/Ndy1/KDM2B/miR-101 pathway leads to the upregulation of Ezh2, which promotes cell proliferation, cell migration and angiogenesis.

Overexpression of Ndy1/KDM2B is sufficient to induce Ezh2 through the mechanisms of miR-101. During senescence in passaged mouse embryo fibroblasts (MEFs), both Ndy1/KDM2B and Ezh2 were downregulated, and the downregulation of Ndy1/KDM2B was envisioned to relieve repression of miR-101, which in turn inhibits the expression of Ezh2 post-transcriptionally. Further data however, refuted this hypothesis by showing that the downregulation of Ezh2 during senescence is independent of miR-101. Moreover, the knockdown of Ndy1/KDM2B in early passage MEFs did not significantly upregulate miR-101 or downregulate Ezh2. To address the physiological role of the signaling module Ndy1/KDM2B/miR-101/Ezh2, early passage MEFs were treated with serum or with several growth factors, including FGF-2, IGF-1, PDGF, VEGF, EGF and TNFα. Further, early passage MEFs were treated with H₂O₂, or antioxidants, including NAC and Trolox. The results herein showed that whereas Ndy1/KDM2B was upregulated by both serum and several growth factors, only FGF-2 and to a lesser degree, VEGF upregulated Ezh2 by repressing miR-101. The interdependence among Ndy1/KDM2B, miR-101 and Ezh2 in FGF-2-stimulated cells was confirmed by the knockdown of Ndy1/KDM2B.

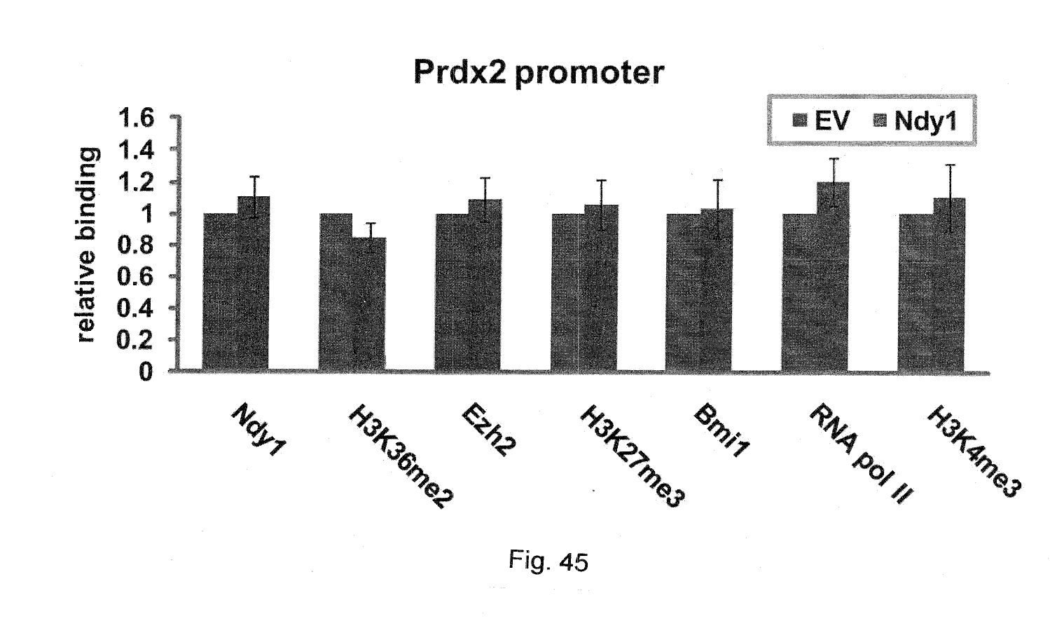

To determine the mechanism by which Ndy1/KDM2B represses the expression of miR-101, chromatin immunoprecipitation (ChIP) was employed, focusing on a DNA region that includes miR-101 and approximately 2 kb upstream of the miR-101 transcriptional start site (FIG. 45). Transient transfection of miR-101-luciferase reporter constructs confirmed that this DNA region is sufficient to sense the repressive activity of Ndy1/KDM2B. The results revealed that Ndy1/KDM2B binds segments of this region, and that binding to this region correlates with a decrease in the abundance of the H3K36me2 marks and with Ezh2 binding and with an increase in the abundance of the H3K27me3 marks. Further, Ndy1/KDM2B binding correlates with binding of Bmi1, a component of the PRC1 complex, and with a decrease in the binding of RNA Pol II. These data are similar to the examples herein describing the mechanism by which Ndy1/KDM2B represses the INK4A/Arf locus. The co-localization of Ndy1/KDM2B and Ezh2 in both loci (miR-101 and INK4A/Arf) is reminiscent of a similar recent finding showing that the enzymatically inactive jumonji domain protein Jmj or Jarid2 co-localizes with Ezh2 on Ezh2 targets and that the co-localization is due to the ability of Jarid2 to promote PRC2 recruitment to target genes. Jarid2 was also shown to modulate the activity of its partner, Ezh2. However, it is not clear whether it stimulates or inhibits its activity, because whereas some studies showed that it inhibits it, other studies showed that it stimulates it. Given that Ndy1/KDM2B also interacts with Ezh2, it is here envisioned that these proteins co-localize with Ezh2 because it is a component of the Jarid2/Ezh2 complex. Further studies will address whether Ndy1/KDM2B also modulates the activity of Ezh2 either by cooperating with Jarid2 or by antagonizing it.

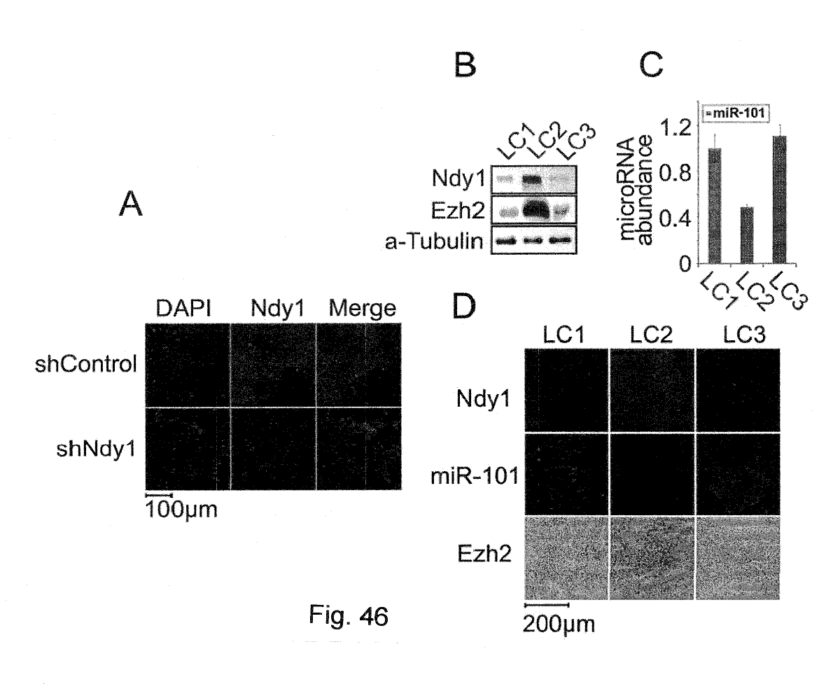

Further, analyses of levels of miR-101, Ndy1 and Ezh2 in three lung tumor samples show the interdependence of expression as shown in FIG. 46, supporting utility of methods herein as cancer diagnostics.

What is claimed is:

1. A pharmaceutical composition for inhibiting immortalization and stimulating differentiation of a cell comprising an effective dose of at least one mammalian short form Ndy protein-related composition selected from the group of: a short form Ndy1 protein; a short form Ndy2 protein; a vector encoding a short form Ndy1 nucleotide sequence; a vector encoding a short form Ndy2 nucleotide sequence; a modulator of short form Ndy1 expression; a modulator of short form Ndy2 expression; wherein the short form Ndy protein, vector and modulator function to increase cellular amount or activity of short form Ndy protein lacking a functional JmjC domain thereby lacking demethylase activity, wherein the short form Ndy protein further inhibits histone demethylase Ndy long form expression or activity.

2. The pharmaceutical composition according to claim 1, further comprising a pharmaceutical buffer.

3. The pharmaceutical composition according to claim 1 further comprising each of a long form and a short form Ndy protein-related compositions in an effective ratio to regulate amount of histone demethylation and thereby regulate gene expression, wherein a higher ratio of long form to short form promotes immortalization, and a lower ratio promotes differentiation and senescence.

4. The pharmaceutical composition according to claim 3, wherein immortalization is promoted by a ratio of greater than about 1:1, about 2:1, about 5:1, or about 10:1 of long form to short form, and wherein differentiation and senescence are promoted by a ratio of less than about 1:1, about 1:2, about 1:5, or about 1:10 of long form to short form.

5. A method of immortalizing a cell comprising:
contacting the cell with a vector carrying a nucleotide sequence selected from the group encoding: an Ndy1 gene operably linked to regulatory signals promoting expression of Ndy1 gene, wherein the Ndy1 gene includes a functional JmjC domain; an Ndy2 gene operably linked to regulatory signals to promote expression of the Ndy2 gene, wherein the Ndy2 gene includes a functional JmjC domain; and a negative modulator of expression of a short form Ndy1 and/or Ndy2 gene; and,
culturing and measuring immortalization of the cell during passaging and storage.

6. The method according to claim 5, wherein prior to contacting, the cell is obtained in vivo from a subject suffering from a senescence condition.

7. The method according to claim 6, further comprising after culturing, implanting the cell in vivo.

8. The method according to claim 6, wherein the senescence condition is neurological, muscular, hematopoietic, or dermatological.

9. The method according to claim 8, wherein the condition is selected from Alzheimers, pre-Alzheimers, amnesia, psychosis, muscular dystrophy, myotonic dystrophy, sickle cell anemia, thallasemia, and progeria.

10. A method of promoting at least one of cell differentiation and cell senescence, the method comprising:
contacting the cell with a vector carrying a nucleotide sequence selected from the group of: a short form of an Ndy1 gene operably linked to regulatory signals to promote expression of the Ndy1 gene, wherein the Ndy1 gene lacks a functional JmjC domain; a short form of an Ndy2 gene operably linked to regulatory signals to promote expression of the Ndy2 gene, wherein the Ndy2 gene lacks information encoding a functional JmjC domain; and a modulator that upregulates expression of at least one of the short form Ndy1 and the short form Ndy2 gene; and,
culturing the cell and analyzing an amount of at least one of differentiation and senescence in cultured amplified cells.

11. The method according to claim 10, further comprising prior to contacting, obtaining the cell from a subject suffering from a cancer or a neoplastic condition.

12. The method according to claim 10, further comprising after culturing, implanting the cells in vivo.

13. The method according to claim 11, wherein the cancer is a hematopoietic condition selected from a leukemia and a lymphoma.

14. The method according to claim 11, wherein the cancer is selected from the group of prostate, testicular cancer, breast, colon, ovarian, bladder, transitional cell carcinoma, pancreatic, esophageal, lung, brain, melanoma, and basal cell carcinoma.

15. The method according to claim 11, further comprising treating the subject with an anti-tumor agent or procedure.

16. The method according to claim 15, wherein the anti-tumor agent or procedure is selected from the group of radiation, thermal disruption, and angiogenesis inhibition.

17. A method of obtaining an anti-Ndy antibody comprising:
contacting an animal with a peptide 907-KMRRKRRLVNKELSKC-921 (SEQ ID NO:2) or a fragment or analog thereof for a time sufficient for a serum sample from the animal to indicate increasing titers of the antibody that functions to bind to an Ndy protein; and,
analyzing amount of antibody in serum recovered from the animal at a time of optimal antibody production, thereby obtaining the anti-Ndy antibody.

18. The method according to claim 17, wherein the fragment is at least four amino acids to seven amino acids in length.

19. The method according to claim 17, wherein the antibody recognizes and binds to an Ndy protein from a mammal.

20. A method of prognosing or diagnosing a cell or tissue for susceptibility to a cancer, the method comprising:
contacting a test sample comprising the cell, tissue or an extract thereof with an anti-Ndy antibody or a nucleotide sequence encoding a portion of an Ndy gene;
observing amount of binding of the antibody or the nucleic acid to the test sample; and,
analyzing the amount in comparison to a control sample from a normal cell or tissue known to be negative for the cancer, wherein an greater amount of binding of the Ndy antigen or nucleic acid to the test sample in comparison to the control samples provides a prognosis or a diagnosis of susceptibility to cancer.

21. The method according to claim 20, further comprising determining extent in the amount of a ratio of Ndy long form compared to Ndy short form, wherein a greater amount of the long form compared to the short form is a prognosis or diagnosis of susceptibility to cancer.

22. A method of identifying from a library of compounds a compound capable of binding to and inhibiting activity of an Ndy protein, the method comprising:
contacting the compound to a first cell having an Ndy retroviral construct encoding a long Ndy form having a JmjC domain; and,
identifying the compound by observing a differentiation morphology of the first cell in comparison with that of a second cell which is a control having the identical Ndy retroviral construct and not so contacted with the compound, and further in comparison with a third cell which is a control identically contacted with the compound and lacking the retroviral construct, thereby identifying the compound as capable of binding to and inhibiting activity of the Ndy protein.

23. The method according to claim 22, wherein each of the first cell, second cell and third cell is a plurality of cells, each in culture in a well of a multi-well culture dish.

24. The method according to claim 28, wherein observing comprises measuring a marker of differentiation by at least one parameter which is immunologic, colorimetric, fluorimetric, fluorescent, radioactive, or enzymatic.

25. A method of treating a subject having a cancer selected from a breast cancer, a testicular cancer, a leukemia and a lymphoma, the method comprising: contacting the subject with a vector carrying an siRNA that inhibits expression of an Ndy protein, wherein the Ndy protein comprises a JmjC domain; and, measuring inhibition by the siRNA of expression of endogenous Ndy protein and function or activity of the JmjC domain, thereby treating the subject.

26. A method of identifying a compound capable of binding to and inhibiting activity of an Ndy protein, the method comprising:
contacting the compound to a first sample of an Ndy protein in an in vitro assay of an enzymatic reaction comprising at least one methylated substrate, and under conditions suitable for histone demethylation, wherein the Ndy protein comprises a JmjC domain; and,
observing inhibition by the compound of an amount of enzymatic reaction product demethylated substrate, wherein the compound is identified as inhibiting the amount produced in the first sample, compared to that of a second control sample having identical Ndy protein and not so contacted with the compound, wherein the compound decreases amount of demethylated product in the first sample compared to the second sample, thereby identifying the compound as inhibiting activity of the Ndy protein.

27. The method according to claim 26 further comprising observing a third sample which is a control comprising substrate and lacking the Ndy protein, wherein the third sample is a control for determining spontaneous non-enzymatic background demethylation.

28. The method according to claim 26, wherein Ndy is a long form of Ndy1 or Ndy2.

29. The method according to claim 26, further comprising prior to contacting, preparing the sample of Ndy protein from at least one of the group of: a crude cell extract; an enriched fraction cell extract by preparative immunoprecipitation; and a bacterially produced recombinant protein.

30. The method according to claim 26, the compound is present in a plurality of compositions in a sibling pool, and contacting the compound to the protein further comprises a plurality of samples in a high throughput multi-well format.

31. The method according to claim 26, wherein the methylated substrate is bulk histone and observing comprises performing a Western blot of an electrophoretogram.

32. The method according to claim 26, wherein the methylated substrate is a di-methylated or a tri-methylated isolated synthetic peptide and observing is measuring a change in fluorescence of product formaldehyde by a glutathione-independent formaldehyde dehydrogenase which reduces NAD⁺ to NADH.

33. The method according to claim 26, wherein conditions suitable for histone demethylation comprise presence of α-ketoglutarate and an iron salt.

34. The method according to claim 33, wherein the di-methylated or tri-methylated isolated synthetic peptide is at least one of ART-K(me₃)-QTARKST and ATGGV-K(me₂)-KPHRY.

35. The method according to claim 26, further comprising observing anti-cancer activity of the compound.

36. A method of treating a cancer or senescence condition of a cell comprising formulating a composition comprising an Ndy amino acid sequence or a nucleotide sequence encoding an Ndy amino acid in a pharmaceutically acceptable buffer or salt, and contacting the cell with the composition.

37. The method according to claim 36, further comprising formulating the composition in an effective dose.

38. A method for inhibiting growth of cells, comprising contacting the cells with an siRNA capable of inhibiting Ndy1 expression.

39. The method according to claim 38, wherein the siRNA comprises nucleotide sequence 1433-GUGGACUCACCUUACCGAAUU-1454 (SEQ ID NO:1) or a portion thereof.


SEQUENCE LISTING (86 sequences)

SEQ ID NO: 1 - RNA, 21 nt, Rattus norvegicus
guggacucaccuuaccgaauu

SEQ ID NO: 2 - protein, 16 aa, Rattus norvegicus
KMRRKRRLVNKELSKC

SEQ ID NO: 3 - DNA, 21 bp, Rattus norvegicus
gggcaggagtgttgacaatta

SEQ ID NO: 4 - DNA, 20 bp, Rattus norvegicus
catccttgtcctggagctaa

SEQ ID NO: 5 - DNA, 20 bp, Mus musculus
gactttgcaaacggatctgc

SEQ ID NO: 6 - DNA, 20 bp, Mus musculus
cgccttagaaatggtccaga

SEQ ID NO: 7 - DNA, 20 bp, Homo sapiens
ctgcctaagtgttggtgcaa

SEQ ID NO: 8 - DNA, 21 bp, Homo sapiens
caggaaaattggcaatctcaa

SEQ ID NO: 9 - DNA, 20 bp, Mus musculus
catccctggagtggtttctt

SEQ ID NO: 10 - DNA, 20 bp, Mus musculus
gcagagagggaatgtgtcgt

SEQ ID NO: 11 - DNA, 30 bp, Rattus norvegicus
cccaaagtgaaaaagctgcatagcttcaac

SEQ ID NO: 12 - DNA, 30 bp, Rattus norvegicus
gttgaagctatgcagctttttcactttggg

SEQ ID NO: 13 - DNA, 30 bp, Rattus norvegicus
cgaacaacagcaggagcgccagtgctgccc

SEQ ID NO: 14 - DNA, 30 bp, Rattus norvegicus
gggcagcactggcgctcctgctgttgttcg

SEQ ID NO: 15 - DNA, 30 bp, Rattus norvegicus
gtgctgccccacaccggcaagaccgggaaa

SEQ ID NO: 16 - DNA, 30 bp, Rattus norvegicus
tttcccggtcttgccggtgtggggcagcac

SEQ ID NO: 17 - DNA, 30 bp, Rattus norvegicus
cggcactcgctgggactggatgatggagca

SEQ ID NO: 18 - DNA, 30 bp, Rattus norvegicus
tgctccatcatccagtcccagcgagtgccg

SEQ ID NO: 19 - DNA, 30 bp, Rattus norvegicus
ctgcccctggatgatgacctgaaccgctgc

SEQ ID NO: 20 - DNA, 30 bp, Rattus norvegicus
gcagcggttcaggtcatcatccaggggcag

SEQ ID NO: 21 - DNA, 24 bp, Rattus norvegicus
ctagtcaagggagacaggctgtcg

SEQ ID NO: 22 - DNA, 38 bp, Rattus norvegicus
ggaggcacctccgtgtgggcccatgtgttccgtggtgg

SEQ ID NO: 23 - DNA, 35 bp, Rattus norvegicus
cccttcaggttggatctatgcggtttatacgcctg

SEQ ID NO: 24 - DNA, 44 bp, Rattus norvegicus
cgaggctgctatactgacttcgctgtggattttggaggtacttc

SEQ ID NO: 25 - DNA, 20 bp, Mus musculus
agacaccagaggcacagagg

SEQ ID NO: 26 - DNA, 20 bp, Mus musculus
cacagtgggacgcttgacta

SEQ ID NO: 27 - DNA, 20 bp, Mus musculus
tgtgtccgtcgtggatctga

SEQ ID NO: 28 - DNA, 21 bp, Mus musculus
cctgcttcaccaccttcttga

SEQ ID NO: 29 - DNA, 20 bp, Mus musculus
caagcagggctataccttcg

SEQ ID NO: 30 - DNA, 21 bp, Mus musculus
ggggatgttaaagctatgcaa

SEQ ID NO: 31 - DNA, 3930 bp, Mus musculus
atggaggcagagaaagactctggaagaagattgcgtgcgattgaccgccagagatacgacgagaacgaagacttgtcggacgtggaggaaattgtcagcgtccgtggcttcagcctggaggagaagctacgtagccagttataccagggggacttcgtgcatgctatggaaggcaaagattttaactatgagtacgtacagagagaagctctcagggtccccctggtttttcgggacaaggatggactagggatcaagatgccagaccctgatttcacagtccgagacgtcaaactcctggtggggagccgccgtttggtggatgtcatggacgtcaacacccagaagggtaccgagatgagcatgtcccagtttgtgcgctactacgagacaccagaggcacagcgggataaactgtacaacgtcatcagcctcgagttcagccatacgaagctggagcatctggtcaagcgtcccactgtggtggacctggtcgactgggtggacaacatgtggcctcagcatctaaaggaaaagcagacagaagccaccaatgcccttgcagagatgaagtaccccaaagtgaaaaagtactgtctgatgagcgtgaagggctgtttcactgacttccacattgactttggaggcacctccgtgtggtaccatgtgttccgtggtggcaagatcttttggctgatccctccaaccctgcacaacttggctttgtacgaggagtgggtgctgtctggcaaacagagcgacatctttctgggagaccgcgtggaacgctgccaaagaattgagctgaagcaaggctacacctttttcatcccttccggttggatccatgcggtttatacgcctgtggactctctggtgttcggcgggaacatcctgcatagcttcaacgtgcccatgcagctgcggatctacgagatcgaggacaggacccgggttcagcccaagttccgttaccccttctactatgagatgtgctggtatgtcttggagagatatgtgtactgtgtgacccagcgctcctacctcactcaggaataccagagagaattaatgcttattgatgccccaagaaaaaccagtgtagacggcttttcatccgactcctggctggacatggaggaggagtcctgcgagcagcagccacaggaggaagaggaggaggaggaggacaaggaggaggaaggagatggtgcagataaaacacccaagccacccaccgatgaccccacctcacccaccagcaccccgcccgaagaccaggacagcacagggaagaagcctaaagcccctgccatacggttcctcaagaggacgttgtccaatgagtcagaggaaagtgtcaagtcgacctcgatgcccacggacgatcccaagacgcccacgggctccccggccaccgaggtttctaccaagtggactcaccttaccgaatttgaactgaagggcttgaaagccctggttgaaaagctagagtcccttccggagaataagaagtgtgtccctgagggaatcgaggacccccaggccctcctggaaggtgtaaagaatgtactgaaagagcacgtggatgacgaccccaccctggccatcaccggggtccctgtggtcagctggccaaagaaaactgcaaagaaccgggtggtgggtcggcctaagggcaagttgggcccggcctcagcggtgaagttggctgccaaccgaacaacagcaggagctcgcaggcgccggacgcgatgccgcaagtgcgaggcctgcctgcggacggagtgtggagagtgccacttttgcaaggacatgaagaagtttggaggtcctgggcgcatgaagcagagctgcatcatgcggcagtgcatcgcgccagtgctgccccacaccgccgtgtgccttgtgtgtggcgaggcagggaaggaggacacagtggaagaggaagaaggcaagtttaacctcatgctcatggaatgctccatctgcaacgagatcatccaccctggatgccttaagattaaggaatcggagggtgtggtcaacgatgagcttcccaactgctgggagtgtccgaagtgtaaccatgccggcaagaccgggaaacaaaagcgtggccctggctttaagtatgcctccaacctgcctggctccttgctcaaggagcagaagatgaaccgggacaacaaggaagggcaagagcctgccaagcggagaagtgagtgtgaagaggctccccgtcggaggtcagacgagcaccccaaaaaggtgcctgcagatggcatcctccgccgaaagtctgatgatgtgcacctgaggaggaagcggaaatacgagaagccccaagagctgagtggacgcaagcgagcctcgtcgcttcaaacgtcccccggttcctcctctcacctctcgccgaggccccctctaggcagcagtctcagcccttggtggagatccagtctcacttacttccagcagcagctaaaacctggcaaagaagataagcttttcaggaaaaagcggcggtcctggaagaacgctgaggatcgtctgtcactggccaacaagccccttcggcgctttaagcaggaaccggaggacgatctgcctgaggcacctcctaagacccgggagagtgatcagtcacgttccagctcacccactgctggtcccagcactgagggagctgaaggcccagaagagaagaaaaaggtgaagatgcgccggaagcggcgacttgttaacaaggagctgagcaaagagctaagcaaggagctcaaccatgagatccaaaagacggagagcaccctggctcacgagagccagcagcccatcaagtcagagcctgagagcgagaacgacgagcccaagaggcccttaagccactgcgagcgcccccaccgcttcagcaaagggctcaacggcacacctcgggagctgcggcactcgctgggacctggcctgcgtagtccacctcgtgttatgtcccggcccccgccctctgcatccccacccaagtgcatccagatggagcgtcacgtgatccggccaccgcccatcagccccccacctgactcgctgcccctggatgatggagcagcccacgtcatgcatagggaggtgtggatggcagtcttcagctacctcagccaccgagacctgtgtgtctgcatgcgggtctgcaggacctggaaccgctggtgctgcgataagcggttgtggacccgcatcgacctgaaccgctgcaagtccatcacacccctgatgctgagcggtatcatccggcgacagcctgtctcccttgacctcagttggaccaacatctccaagaagcagctgagttggctcatcaaccggttgcctgggctccgagacttggtgctgtcaggctgctcatggatcgctgtctcagccctctgtagctccagttgtccattgctccggaccctggatgtccagtgggtagaaggactaaaggatgcccagatgagggatctcctgtctccacccacagacaacaggccaggtcagatggacaatcggagcaagctccggaacattgtagagctgcgcctagctggcctggacatcacagatgtctccctgcggctcattattcgccatatgcccctgctctcgaagctccaactcagttactgtaaccacatcaatgaccagtccatcaacctgctcactgccgtcggcaccaccacccgagactcgctgacagaggtcaacctatcagactgtaataaggtaactgacctgtgcctgtccttcttcaaacgctgtggaaatatctgtcatattgacctgaggtactgcaagcaagtcaccaaggaaggctgtgagcaattcatagctgaaatgtctgtgagtgtccaatttgggcaagtggaagagaaactcctgcaaaaactaagttag

SEQ ID NO: 32 - DNA, 3912 bp, Mus musculus
atgaggcagctgctgcgtgcgattgaccgccagagatacgacgagaacgaagacttgtcggacgtggaggaaattgtcagcgtccgtggcttcagcctggaggagaagctacgtagccagttataccagggggacttcgtgcatgctatggaaggcaaagattttaactatgagtacgtacagagagaagctctcagggtccccctggtttttcgggacaaggatggactagggatcaagatgccagaccctgatttcacagtccgagacgtcaaactcctggtggggagccgccgtttggtggatgtcatggacgtcaacacccagaagggtaccgagatgagcatgtcccagtttgtgcgctactacgagacaccagaggcacagcgggataaactgtacaacgtcatcagcctcgagttcagccatacgaagctggagcatctggtcaagcgtcccactgtggtggacctggtcgactgggtggacaacatgtggcctcagcatctaaaggaaaagcagacagaagccaccaatgcccttgcagagatgaagtaccccaaagtgaaaaagtactgtctgatgagcgtgaagggctgtttcactgacttccacattgactttggaggcacctccgtgtggtaccatgtgttccgtggtggcaagatcttttggctgatccctccaaccctgcacaacttggctttgtacgaggagtgggtgctgtctggcaaacagagcgacatctttctgggagaccgcgtggaacgctgccaaagaattgagctgaagcaaggctacacctttttcatcccttccggttggatccatgcggtttatacgcctgtggactctctggtgttcggcgggaacatcctgcatagcttcaacgtgcccatgcagctgcggatctacgagatcgaggacaggacccgggttcagcccaagttccgttaccccttctactatgagatgtgctggtatgtcttggagagatatgtgtactgtgtgacccagcgctcctacctcactcaggaataccagagagaattaatgcttattgatgccccaagaaaaaccagtgtagacggcttttcatccgactcctggctggacatggaggaggagtcctgcgagcagcagccacaggaggaagaggaggaggaggaggacaaggaggaggaaggagatggtgcagataaaacacccaagccacccaccgatgaccccacctcacccaccagcaccccgcccgaagaccaggacagcacagggaagaagcctaaagcccctgccatacggttcctcaagaggacgttgtccaatgagtcagaggaaagtgtcaagtcgacctcgatgcccacggacgatcccaagacgcccacgggctccccggccaccgaggtttctaccaagtggactcaccttaccgaatttgaactgaagggcttgaaagccctggttgaaaagctagagtcccttccggagaataagaagtgtgtccctgagggaatcgaggacccccaggccctcctggaaggtgtaaagaatgtactgaaagagcacgtggatgacgaccccaccctggccatcaccggggtccctgtggtcagctggccaaagaaaactgcaaagaaccgggtggtgggtcggcctaagggcaagttgggcccggcctcagcggtgaagttggctgccaaccgaacaacagcaggagctcgcaggcgccggacgcgatgccgcaagtgcgaggcctgcctgcggacggagtgtggagagtgccacttttgcaaggacatgaagaagtttggaggtcctgggcgcatgaagcagagctgcatcatgcggcagtgcatcgcgccagtgctgccccacaccgccgtgtgccttgtgtgtggcgaggcagggaaggaggacacagtggaagaggaagaaggcaagtttaacctcatgctcatggaatgctccatctgcaacgagatcatccaccctggatgccttaagattaaggaatcggagggtgtggtcaacgatgagcttcccaactgctgggagtgtccgaagtgtaaccatgccggcaagaccgggaaacaaaagcgtggccctggctttaagtatgcctccaacctgcctggctccttgctcaaggagcagaagatgaaccgggacaacaaggaagggcaagagcctgccaagcggagaagtgagtgtgaagaggctccccgtcggaggtcagacgagcaccccaaaaaggtgcctgcagatggcatcctccgccgaaagtctgatgatgtgcacctgaggaggaagcggaaatacgagaagccccaagagctgagtggacgcaagcgagcctcgtcgcttcaaacgtcccccggttcctcctctcacctctcgccgaggccccctctaggcagcagtctcagcccttggtggagatccagtctcacttacttccagcagcagctaaaacctggcaaagaagataagcttttcaggaaaaagcggcggtcctggaagaacgctgaggatcgtctgtcactggccaacaagccccttcggcgctttaagcaggaaccggaggacgatctgcctgaggcacctcctaagacccgggagagtgatcagtcacgttccagctcacccactgctggtcccagcactgagggagctgaaggcccagaagagaagaaaaaggtgaagatgcgccggaagcggcgacttgttaacaaggagctgagcaaagagctaagcaaggagctcaaccatgagatccaaaagacggagagcaccctggctcacgagagccagcagcccatcaagtcagagcctgagagcgagaacgacgagcccaagaggcccttaagccactgcgagcgcccccaccgcttcagcaaagggctcaacggcacacctcgggagctgcggcactcgctgggacctggcctgcgtagtccacctcgtgttatgtcccggcccccgccctctgcatccccacccaagtgcatccagatggagcgtcacgtgatccggccaccgcccatcagccccccacctgactcgctgcccctggatgatggagcagcccacgtcatgcatagggaggtgtggatggcagtcttcagctacctcagccaccgagacctgtgtgtctgcatgcgggtctgcaggacctggaaccgctggtgctgcgataagcggttgtggacccgcatcgacctgaaccgctgcaagtccatcacacccctgatgctgagcggtatcatccggcgacagcctgtctcccttgacctcagttggaccaacatctccaagaagcagctgagttggctcatcaaccggttgcctgggctccgagacttggtgctgtcaggctgctcatggatcgctgtctcagccctctgtagctccagttgtccattgctccggaccctggatgtccagtgggtagaaggactaaaggatgcccagatgagggatctcctgtctccacccacagacaacaggccaggtcagatggacaatcggagcaagctccggaacattgtagagctgcgcctagctggcctggacatcacagatgtctccctgcggctcattattcgccatatgcccctgctctcgaagctccaactcagttactgtaaccacatcaatgaccagtccatcaacctgctcactgccgtcggcaccaccacccgagactcgctgacagaggtcaacctatcagactgtaataaggtaactgacctgtgcctgtccttcttcaaacgctgtggaaatatctgtcatattgacctgaggtactgcaagcaagtcaccaaggaaggctgtgagcaattcatagctgaaatgtctgtgagtgtccaatttgggcaagtggaagagaaactcctgcaaaaactaagttag

SEQ ID NO: 33 - DNA, 2331 bp, Mus musculus
atggccatgtccgtgagcgccgaggacgacgactatgaatcggagcccgaccagaaccgggtggtgggtcggcctaagggcaagttgggcccggcctcagcggtgaagttggctgccaaccgaacaacagcaggagctcgcaggcgccggacgcgatgccgcaagtgcgaggcctgcctgcggacggagtgtggagagtgccacttttgcaaggacatgaagaagtttggaggtcctgggcgcatgaagcagagctgcatcatgcggcagtgcatcgcgccagtgctgccccacaccgccgtgtgccttgtgtgtggcgaggcagggaaggaggacacagtggaagaggaagaaggcaagtttaacctcatgctcatggaatgctccatctgcaacgagatcatccaccctggatgccttaagattaaggaatcggagggtgtggtcaacgatgagcttcccaactgctgggagtgtccgaagtgtaaccatgccggcaagaccgggaaacaaaagcgtggccctggctttaagtatgcctccaacctgcctggctccttgctcaaggagcagaagatgaaccgggacaacaaggaagggcaagagcctgccaagcggagaagtgagtgtgaagaggctccccgtcggaggtcagacgagcaccccaaaaaggtgcctgcagatggcatcctccgccgaaagtctgatgatgtgcacctgaggaggaagcggaaatacgagaagccccaagagctgagtggacgcaagcgagcctcgtcgcttcaaacgtcccccggttcctcctctcacctctcgccgaggccccctctaggcagcagtctcagcccttggtggagatccagtctcacttacttccagcagcagctaaaacctggcaaagaagataagcttttcaggaaaaagcggcggtcctggaagaacgctgaggatcgtctgtcactggccaacaagccccttcggcgctttaagcaggaaccggaggacgatctgcctgaggcacctcctaagacccgggagagtgatcagtcacgttccagctcacccactgctggtcccagcactgagggagctgaaggcccagaagagaagaaaaaggtgaagatgcgccggaagcggcgacttgttaacaaggagctgagcaaagagctaagcaaggagctcaaccatgagatccaaaagacggagagcaccctggctcacgagagccagcagcccatcaagtcagagcctgagagcgagaacgacgagcccaagaggcccttaagccactgcgagcgcccccaccgcttcagcaaagggctcaacggcacacctcgggagctgcggcactcgctgggacctggcctgcgtagtccacctcgtgttatgtcccggcccccgccctctgcatccccacccaagtgcatccagatggagcgtcacgtgatccggccaccgcccatcagccccccacctgactcgctgcccctggatgatggagcagcccacgtcatgcatagggaggtgtggatggcagtcttcagctacctcagccaccgagacctgtgtgtctgcatgcgggtctgcaggacctggaaccgctggtgctgcgataagcggttgtggacccgcatcgacctgaaccgctgcaagtccatcacacccctgatgctgagcggtatcatccggcgacagcctgtctcccttgacctcagttggaccaacatctccaagaagcagctgagttggctcatcaaccggttgcctgggctccgagacttggtgctgtcaggctgctcatggatcgctgtctcagccctctgtagctccagttgtccattgctccggaccctggatgtccagtgggtagaaggactaaaggatgcccagatgagggatctcctgtctccacccacagacaacaggccaggtcagatggacaatcggagcaagctccggaacattgtagagctgcgcctagctggcctggacatcacagatgtctccctgcggctcattattcgccatatgcccctgctctcgaagctccaactcagttactgtaaccacatcaatgaccagtccatcaacctgctcactgccgtcggcaccaccacccgagactcgctgacagaggtcaacctatcagactgtaataaggtaactgacctgtgcctgtccttcttcaaacgctgtggaaatatctgtcatattgacctgaggtactgcaagcaagtcaccaaggaaggctgtgagcaattcatagctgaaatgtctgtgagtgtccaatttgggcaagtggaagagaaactcctgcaaaaactaagttag

SEQ ID NO: 34 - DNA, 4011 bp, Homo sapiens
atggcgggtccgcaaatggggggatctgcagaggatcaccccccacgaaaaagacatgcagcagaaaagcaaaaaaagaaaacagttatatatacaaaatgctttgaatttgagtcggccacacagcgcccgattgaccgccagcgatacgacgagaacgaggacttgtcggacgtggaggagatcgtcagcgtccgcggcttcagcctggaggagaagcttcgcagccagctgtaccagggggacttcgtgcacgccatggagggcaaagatttcaactatgagtacgtacagagagaagctctcagggttcccctgatatttcgagaaaaggatggactgggaattaagatgcctgaccctgatttcacagtccgagacgtcaaactcctagtggggagccggcggcttgtggacgtgatggatgtgaacacccagaagggcacggagatgagcatgtcccagtttgtgcgttactacgagacgcccgaggcccagcgggacaagctgtacaacgtcatcagcctagagttcagccacaccaagctggagcacttggtcaagcgtccgactgtggtagacctggtggactgggtggacaacatgtggccccagcatctgaaggagaagcagacagaagccacgaacgccattgcagagatgaagtacccgaaagtgaaaaagtactgtctgatgagcgtgaaaggttgtttcaccgacttccacatcgactttggaggcacttccgtttggtaccatgttttccggggtgggaagattttttggctgattcctccaacgctgcacaatttggcgctgtacgaggagtgggtgctgtcaggcaaacagagtgacatctttctgggagaccgtgtggaacgatgccaaagaattgagctgaagcagggctacacatttttcatcccttccggttggatccatgccgtctacacccctgtagactctttggtgttcggcggaaacatcctgcacagctttaacgtgcccatgcagctgcggatctacgagatcgaggacaggacgcgggtgcagcccaaattccgttaccccttctactatgagatgtgctggtatgtcctggagagatacgtgtactgtgtgacccagcgctcccacctcactcaggaataccagagggagtcgatgcttattgatgccccgaggaagcccagcatagacggcttctcttcggattcctggctggagatggaggaggaggcctgtgatcagcagcctcaggaggaggaggagaaggacgaggagggcgagggcagggacagggcacccaaaccgcccaccgatggctccacttcacccaccagcacgccctctgaggaccaggaggccctcgggaagaagcccaaagcacctgccctgcgattcctcaaaaggactttgtctaatgagtcggaggaaagtgtgaagtccaccacattggccgtagactaccccaagacccccaccggctctcccgccacggaggtctctgccaaatggacccatctcactgagtttgaactgaagggcctgaaagctctggtggagaaactggaatccctcccggagaacaagaagtgtgtccccgagggcatcgaggacccccaggcactcctggagggtgtgaagaacgtcctgaaggagcacgcagatgatgaccctagtctggccatcactggggtccctgtggtgacttggccaaagaagactccaaagaaccgggctgtgggtcggcccaaggggaagctgggcccggcctccgcggtgaagttggccgccaaccggacaacggcaggagctcggcggcgccggacgcgatgccgcaagtgcgaggcctgcctgcggaccgagtgcggagagtgccacttctgcaaggacatgaagaagttcgggggccccgggcgcatgaagcagagctgcatcatgcggcagtgcatcgcgccagtgctgccccacaccgccgtgtgccttgtgtgtggcgaggcggggaaggaagacacggtggaagaggaggaaggcaagtttaacctcatgctcatggagtgctccatctgcaatgaaatcatccaccctggatgccttaagattaaggagtcagagggtgtggtcaacgacgagcttccaaactgctgggagtgtccgaagtgtaaccacgccggcaagaccgggaaacaaaagcgtggccctggctttaagtacgcctccaacctgcccggctccctgctcaaggagcagaagatgaaccgggacaacaaggaagggcaggaacctgccaagcggaggagtgagtgtgaggaggcgccccggcgcaggtcggatgagcactcgaagaaggtgccgccggacggccttctgcgcagaaagtctgacgacgtgcacctgaggaagaagcggaaatacgagaagccccaggagctgagtggacgcaagcgggcctcatcgcttcaaacgtcccccggttcctcctctcacctctcgccgaggccccctctaggcagcagcctcagcccctggtggagatccagtctcacttacttccagcagcagctcaaacctggcaaagaagataagcttttcaggaaaaagcggcggtcctggaagaacgccgaggaccgcatggcgctggccaacaagcccctccggcgcttcaagcaggaacccgaggacgaactgcccgaggcgccccccaagaccagggagagcgaccactcccgctccagctcccccaccgcgggacccagcaccgaaggggccgagggcccggaggagaagaagaaggtgaagatgcgccggaagcggcggcttcccaacaaggagctgagcagggagctgagcaaggagctcaaccacgagatccagaggacggagaacagcctggccaacgagaaccagcagcccatcaagtcggagcctgagagcgagggcgaggagcccaagcggcccccgggcatctgcgagcgtccccaccgcttcagcaaggggctcaacggcaccccccgggagctgcggcaccagctggggcccagcctgcgcagcccgccccgtgtcatctcccggcccccaccctccgtgtccccgcccaagtgtatccagatggagcgccatgtgatccggccaccccccatcagccccccgcctgactcgctacccctggacgatggggcagcccacgtcatgcacagggaggtgtggatggccgtcttcagctacctcagccaccaagacctgtgtgtgtgcatgcgggtctgcaggacctggaaccgctggtgctgcgataagcggttgtggacccgcattgacctgaaccactgcaagtctatcacacccctgatgctgagtggcatcatccggcgacagcccgtctccctcgacctcagctggaccaatatctccaagaagcagctgagctggctcatcaaccggctgcctgggctccgggacttggtgctgtcaggctgctcatggatcgcggtctcggccctttgcagctccagttgtccgctgctccggaccctggatgtccagtgggtggagggactaaaggatgcccagatgcgggatctcctgtccccgcccacagacaacaggccaggtcagatggacaatcggagcaagctccggaacatcgtggagctgcgcctggcaggcctggacatcacagatgcctccctgcggctcatcatccgccacatgcccctgctctccaagctccacctcagttactgtaaccacgtcaccgaccagtctatcaacctgctcactgctgttggcaccaccacccgagactccttaaccgagatcaacctgtctgactgcaataaggtcactgatcagtgcctgtccttcttcaaacgctgtggaaacatctgtcatattgacctgaggtactgcaagcaagtcaccaaggaaggctgtgagcagttcatagccgagatgtctgtgagtgtccagtttgggcaagtagaagaaaaactcctgcaaaaactgagttag

SEQ ID NO: 35 - DNA, 3798 bp, Homo sapiens
atggaggcagagaaagactctggaagaagattgcgcccgattgaccgccagcgatacgacgagaacgaggacttgtcggacgtggaggagatcgtcagcgtccgcggcttcagcctggaggagaagcttcgcagccagctgtaccagggggacttcgtgcacgccatggagggcaaagatttcaactatgagtacgtacagagagaagctctcagggttcccctgatatttcgagaaaaggatggactgggaattaagatgcctgaccctgatttcacagtccgagacgtcaaactcctagtggggagccggcggcttgtggacgtgatggatgtgaacacccagaagggcacggagatgagcatgtcccagtttgtgcgttactacgagacgcccgaggcccagcgggacaagctgtacaacgtcatcagcctagagttcagccacaccaagctggagcacttggtcaagcgtccgactgtggtagacctggtggactgggtggacaacatgtggccccagcatctgaaggagaagcagacagaagccacgaacgccattgcagagatgaagtacccgaaagtgaaaaagtactgtctgatgagcgtgaaaggttgtttcaccgacttccacatcgactttggaggcacttccgtttggtaccatgttttccggggtgggaagattttttggctgattcctccaacgctgcacaatttggcgctgtacgaggagtgggtgctgtcaggcaaacagagtgacatctttctgggagaccgtgtggaacgatgccaaagaattgagctgaagcagggctacacatttttcatcccttccggttggatccatgccgtctacacccctgtagactctttggtgttcggcggaaacatcctgcacagctttaacgtgcccatgcagctgcggatctacgagatcgaggacaggacgcgggtgcagcccaaattccgttaccccttctactatgagatgtgctggtatgtcctggagagatacgtgtactgtgtgacccagcgctcccacctcactcaggaataccagagggagtcgatgcttattgatgccccgaggaagcccagcatagacggcttctcttcggattcctggctggagatggaggaggaggcctgtgatcagcagcctcaggaggaggaggagaaggacgaggagggcgagggcagggacagggcacccaaaccgcccaccgatggctccacttcacccaccagcacgccctctgaggaccaggaggccctcgggaagaagcccaaagcacctgccctgcgattcctcaaaaggactttgtctaatgagtcggaggaaagtgtgaagtccaccacattggccgtagactaccccaagacccccaccggctctcccgccacggaggtctctgccaaatggacccatctcactgagtttgaactgaagggcctgaaagctctggtggagaaactggaatccctcccggagaacaagaagtgtgtccccgagggcatcgaggacccccaggcactcctggagggtgtgaagaacgtcctgaaggagcacgcagatgatgaccctagtctggccatcactggggtccctgtggtgacttggccaaagaagactccaaagaaccgggctgtgggtcggcccaaggggaagctgggcccggcctccgcggtgaagttggccgccaaccggacaacggcaggagctcggcggcgccggacgcgatgccgcaagtgcgaggcctgcctgcggaccgagtgcggagagtgccacttctgcaaggacatgaagaagttcgggggccccgggcgcatgaagcagagctgcatcatgcggcagtgcatcgcgccagtgctgccccacaccgccgtgtgccttgtgtgtggcgaggcggggaaggaagacacggtggaagaggaggaaggcaagtttaacctcatgctcatggagtgctccatctgcaatgaaatcatccaccctggatgccttaagattaaggagtcagagggtgtggtcaacgacgagcttccaaactgctgggagtgtccgaagtgtaaccacgccggcaagaccgggaaacaaaagcgtggccctggctttaagtacgcctccaacctgcccggctccctgctcaaggagcagaagatgaaccgggacaacaaggaagggcaggaacctgccaagcggaggagtgagtgtgaggaggcgccccggcgcaggtcggatgagcactcgaagaaggtgccgccggacggccttctgcgcagaaagtctgacgacgtgcacctgaggaagaagcggaaatacgagaagccccaggagctgagtggacgcaagcggctcaaacctggcaaagaagataagcttttcaggaaaaagcggcggtcctggaagaacgccgaggaccgcatggcgctggccaacaagcccctccggcgcttcaagcaggaacccgaggacgaactgcccgaggcgccccccaagaccagggagagcgaccactcccgctccagctcccccaccgcgggacccagcaccgaaggggccgagggcccggaggagaagaagaaggtgaagatgcgccggaagcggcggcttcccaacaaggagctgagcagggagctgagcaaggagctcaaccacgagatccagaggacggagaacagcctggccaacgagaaccagcagcccatcaagtcggagcctgagagcgagggcgaggagcccaagcggcccccgggcatctgcgagcgtccccaccgcttcagcaaggggctcaacggcaccccccgggagctgcggcaccagctggggcccagcctgcgcagcccgccccgtgtcatctcccggcccccaccctccgtgtccccgcccaagtgtatccagatggagcgccatgtgatccggccaccccccatcagccccccgcctgactcgctacccctggacgatggggcagcccacgtcatgcacagggaggtgtggatggccgtcttcagctacctcagccaccaagacctgtgtgtgtgcatgcgggtctgcaggacctggaaccgctggtgctgcgataagcggttgtggacccgcattgacctgaaccactgcaagtctatcacacccctgatgctgagtggcatcatccggcgacagcccgtctccctcgacctcagctggaccaatatctccaagaagcagctgagctggctcatcaaccggctgcctgggctccgggacttggtgctgtcaggctgctcatggatcgcggtctcggccctttgcagctccagttgtccgctgctccggaccctggatgtccagtgggtggagggactaaaggatgcccagatgcgggatctcctgtccccgcccacagacaacaggccaggtcagatggacaatcggagcaagctccggaacatcgtggagctgcgcctggcaggcctggacatcacagatgcctccctgcggctcatcatccgccacatgcccctgctctccaagctccacctcagttactgtaaccacgtcaccgaccagtctatcaacctgctcactgctgttggcaccaccacccgagactccttaaccgagatcaacctgtctgactgcaataaggtcactgatcagtgcctgtccttcttcaaacgctgtggaaacatctgtcatattgacctgaggtactgcaagcaagtcaccaaggaaggctgtgagcagttcatagccgagatgtcttctttccaagggagaagctgttcaaccacccgtttgggggatgagtga

SEQ ID NO: 36 - DNA, 2522 bp, Homo sapiens
gccgagctaatttccttctgagccccccaaatgcctcctccacatggcgggtccgcaaatggggggatctgcagaggatcaccccccacgaaaaagacatgcagcagaaaagcaaaaaaagaaaacagttatatatacaaaatgctttgaatttgagtcggccacacagcgcccgattgaccgccagcgatacgacgagaacgaggacttgtcggacgtggaggagatcgtcagcgtccgcggcttcagcctggaggagaagcttcgcagccagctgtaccagggggacttcgtgcacgccatggagggcaaaggatgcctgaccctgatttcacagtccgagacgtcaaactcctagtggggagccggcggcttgtggacgtgatggatgtgaacacccagaagggcacggagatgagcatgtcccagtttgtgcgttactacgagacgcccgaggcccagcgggacaagctgtacaacgtcatcagcctagagttcagccacaccaagctggagcacttggtcaagcgtccgactgtggtagacctggtggactgggtggacaacatgtggccccagcatctgaaggagaagcagacagaagccacgaacgccattgcagagatgaagtacccgaaagtgaaaaagtactgtctgatgagcgtgaaaggttgtttcaccgacttccacatcgactttggaggcacttccgtttggtaccatgttttccggggtgggaagattttttggctgattcctccaacgctgcacaatttggcgctgtacgaggagtgggtgctgtcaggcaaacagagtgacatctttctgggagaccgtgtggaacgatgccaaagaattgagctgaagcagggctacacatttttcatcccttccggttggatccatgccgtctacacccctgtagactctttggtgttcggcggaaacatcctgcacagctttaacgtgcccatgcagctgcggatctacgagatcgaggacaggacgcgggtgcagcccaaattccgttaccccttctactatgagatgtgctggtatgtcctggagagatacgtgtactgtgtgacccagcgctcccacctcactcaggaataccagagggagtcgatgcttattgatgccccgaggaagcccagcatagacggcttctcttcggattcctggctggagatggaggaggaggcctgtgatcagcagcctcaggaggaggaggagaaggacgaggagggcgagggcagggacagggcacccaaaccgcccaccgatggctccacttcacccaccagcacgccctctgaggaccaggaggccctcgggaagaagcccaaagcacctgccctgcgattcctcaaaaggactttgtctaatgagtcggaggaaagtgtgaagtccaccacattggccgtagactaccccaagacccccaccggctctcccgccacggaggtctctgccaaatggacccatctcactgagtttgaactgaagggcctgaaagctctggtggagaaactggaatccctcccggagaacaagaagtgtgtccccgagggcatcgaggacccccaggcactcctggagggtgtgaagaacgtcctgaaggagcacgcagatgatgaccctagtctggccatcactggggtccctgtggtgacttggccaaagaagactccaaagaaccgggctgtgggtcggcccaaggggaagctgggcccggcctccgcggtgaagttggccgccaaccggacaacggcaggagctcggcggcgccggacgcgatgccgcaagtgcgaggcctgcctgcggaccgagtgcggagagtgccacttctgcaaggacatgaagaagttcgggggccccgggcgcatgaagcagagctgcatcatgcggcagtgcatcgcgactgcaataaggtcactgatcagtgcctgtccttcttcaaacgctgtggaaacatctgtcatattgacctgaggtactgcaagcaagtcaccaaggaaggctgtgagcagttcatagccgagatgtctgtgagtgtccagtttgggcaagtagaagaaaaactcctgcaaaaactgagttagtccaaggataagtatgtaaatacggggcgggctctgggaggggagagactttacaaaaatgagggcttttattttccatttggaacgtgggacaacagaccacaacgcaattccattttgcaagtctttccaagggagaagctgttcaaccacccgtttgggggatgagtgagccgacactttcctttggtctttctgaatcgtaactgcactgctttctggaccatttctaaggcggcctttacaagaagacattcctgtcggagaggagggtggacttcggagaaattctcatactgaagcatgagcttaggagtttctgttagtggtagtggtgttttggacacttcattccttgcaacaccgaggttttgggtgttgacataaagtggaccacacaccacatctgctgccgtc

SEQ ID NO: 37 - DNA, 2762 bp, Homo sapiens
gccgagctaatttccttctgagccccccaaatgcctcctccacatggcgggtccgcaaatggggggatctgcagaggatcaccccccacgaaaaagacatgcagcagaaaagcaaaaaaagaaaacagttatatatacaaaatgctttgaatttgagtcggccacacagcgcccgattgaccgccagcgatacgacgagaacgaggacttgtcggacgtggaggagatcgtcagcgtccgcggcttcagcctggaggagaagcttcgcagccagctgtaccagggggacttcgtgcacgccatggagggcaaagatttcaactatgagtacgtacagagagaagctctcagggttcccctgatatttcgagaaaaggatggactgggaattaagatgcctgaccctgatttcacagtccgagacgtcaaactcctagtggggagccggcggcttgtggacgtgatggatgtgaacacccagaagggcacggagatgagcatgtcccagtttgtgcgttactacgagacgcccgaggcccagcgggacaagctgtacaacgtcatcagcctagagttcagccacaccaagctggagcacttggtcaagcgtccgactgtggtagacctggtggactgggtggacaacatgtggccccagcatctgaaggagaagcagacagaagccacgaacgccattgcagagatgaagtacccgaaagtgaaaaagtactgtctgatgagcgtgaaaggttgtttcaccgacttccacatcgactttggaggcacttccgtttggtaccatgttttccggggtgggaagattttttggctgattcctccaacgctgcacaatttggcgctgtacgaggagtgggtgctgtcaggcaaacagagtgacatctttctgggagaccgtgtggaacgatgccaaagaattgagctgaagcagggctacacatttttcatcccttccggttggatccatgccgtctacacccctgtagactctttggtgttcggcggaaacatcctgcacagctttaacgtgcccatgcagctgcggatctacgagatcgaggacaggacgcgggtgcagcccaaattccgttaccccttctactatgagatgtgctggtatgtcctggagagatacgtgtactgtgtgacccagcgctcccacctcactcaggaataccagagggagtcgatgcttattgatgccccgaggaagcccagcatagacggcttctcttcggattcctggctggagatggaggaggaggcctgtgatcagcagcctcaggaggaggaggagaaggacgaggagggcgagggcagggacagggcacccaaaccgcccaccgatggctccacttcacccaccagcacgccctctgaggaccaggaggccctcgggaagaagcccaaagcacctgccctgcgattcctcaaaaggactttgtctaatgagtcggaggaaagtgtgaagtccaccacattggccgtagactaccccaagacccccaccggctctcccgccacggaggtctctgccaaatggacccatctcactgagtttgaactgaagggcctgaaagctctggtggagaaactggaatccctcccggagaacaagaagtgtgtccccgagggcatcgaggacccccaggcactcctggagggtgtgaagaacgtcctgaaggagcacgcagatgatgaccctagtctggccatcactggggtccctgtggtgacttggccaaagaagactccaaagaaccgggctgtgggtcggcccaaggggaagctgggcccggcctccgcggtgaagttggccgccaaccggacaacggcaggagctcggcggcgccggacgcgatgccgcaagtgcgaggcctgcctgcggaccgagtgcggagagtgccacttctgcaaggacatgaagaagttcgggggccccgggcgcatgaagcagagctgcatcatgcggcagtgcatcgcgccagtgctgccccacaccgccgtgtgccttgtgtgtggcgaggcggggaaggaagacacggtggaagaggaggaaggcaagtttaacctcatgctcatggagtgctccatctgcaatgaaatcatccaccctggatgccttaaccgagatcaacctgtctgactgcaataaggtcactgatcagtgcctgtccttcttcaaacgctgtggaaacatctgtcatattgacctgaggtactgcaagcaagtcaccaaggaaggctgtgagcagttcatagccgagatgtctgtgagtgtccagtttgggcaagtagaagaaaaactcctgcaaaaactgagttagtccaaggataagtatgtaaatacggggcgggctctgggaggggagagactttacaaaaatgagggcttttattttccatttggaacgtgggacaacagaccacaacgcaattccattttgcaagtctttccaagggagaagctgttcaaccacccgtttgggggatgagtgagccgacactttcctttggtctttctgaatcgtaactgcactgctttctggaccatttctaaggcggcctttacaagaagacattcctgtcggagaggagggtggacttcggagaaattctcatactgaagcatgagcttaggagtttctgttagtggtagtggtgttttggacacttcattccttgcaacaccgaggttttgggtgttgacataaagtggaccacacaccacatctgctgccgtc

SEQ ID NO: 38 - DNA, 20 bp, Mus musculus
actgctggcaccgtctgatg

SEQ ID NO: 39 - DNA, 24 bp, Mus musculus
tcctgagaaataatctccccacag

SEQ ID NO: 40 - DNA, 22 bp, Mus musculus
agatgagtcaccagagggatgg

SEQ ID NO: 41 - DNA, 23 bp, Mus musculus
tcactcccagagtcactttccag

SEQ ID NO: 42 - DNA, 18 bp, Mus musculus
gtgtgcatgacgtgcggg

SEQ ID NO: 43 - DNA, 22 bp, Mus musculus
gcagttcgaatctgcaccgtag

SEQ ID NO: 44 - DNA, 21 bp, Mus musculus
gctctggctttcgtgaacatg

SEQ ID NO: 45 - DNA, 22 bp, Mus musculus
tcgaatctgcaccgtagttgag

SEQ ID NO: 46 - DNA, 20 bp, Mus musculus
cacagtgggacgcttgacta

SEQ ID NO: 47 - DNA, 20 bp, Mus musculus
tggccatggaaatgctatca

SEQ ID NO: 48 - DNA, 21 bp, Mus musculus
acacctccgaatattgccaca

SEQ ID NO: 49 - DNA, 25 bp, Mus musculus
actattgctgttaaggagacgctga

SEQ ID NO: 50 - DNA, 20 bp, Mus musculus
gcaggtcgtctctggcttct

SEQ ID NO: 51 - RNA, 21 nt, Rattus norvegicus
guggacucaccuuaccgaauu

SEQ ID NO: 52 - RNA, 21 nt, Rattus norvegicus
ccgaggacgacgacuaugauu

SEQ ID NO: 53 - DNA, 21 bp, Mus musculus
aaaaccctctcttggagtggg

SEQ ID NO: 54 - DNA, 22 bp, Mus musculus
gcaggttcttggtcactgtgag

SEQ ID NO: 55 - DNA, 22 bp, Mus musculus
ctccctttgctacccctgagag

SEQ ID NO: 56 - DNA, 23 bp, Mus musculus
ttacttatttcgctcccatccac

SEQ ID NO: 57 - DNA, 24 bp, Mus musculus
cttagagttacagaaagggctgga

SEQ ID NO: 58 - DNA, 24 bp, Mus musculus
gaatttcaaggaagtgctacccta

SEQ ID NO: 59 - DNA, 20 bp, Mus musculus
gtttcaggaaagccaaacca

SEQ ID NO: 60 - DNA, 20 bp, Mus musculus
ggtagcccagggtactgtga

SEQ ID NO: 61 - DNA, 20 bp, Mus musculus
aagggtcaactgtcctgtgg

SEQ ID NO: 62 - DNA, 20 bp, Mus musculus
gaagatactgaggcccacca

SEQ ID NO: 63 - DNA, 20 bp, Mus musculus
cactgcactggaagaggaca

SEQ ID NO: 64 - DNA, 20 bp, Mus musculus
ctgaaggtcctgggttcaaa

SEQ ID NO: 65 - DNA, 24 bp, Mus musculus
ttctgagtttatgctgagttccag

SEQ ID NO: 66 - DNA, 24 bp, Mus musculus
gtactggacagaagggaggattta

SEQ ID NO: 67 - DNA, 22 bp, Mus musculus
gatggagcccggactacagaag

SEQ ID NO: 68 - DNA, 21 bp, Mus musculus
ctgtttcaacgcccagctctc

SEQ ID NO: 69 - DNA, 23 bp, Mus musculus
caaaagttacccgactgcagatg

SEQ ID NO: 70 - DNA, 25 bp, Mus musculus
aaaagaacatcggtttcaacttgac

SEQ ID NO: 71 - DNA, 22 bp, Mus musculus
tgttgcagtttcagaaggcacc

SEQ ID NO: 72 - DNA, 21 bp, Mus musculus
gaactctttcggtcgtacccc

SEQ ID NO: 73 - DNA, 20 bp, Mus musculus
gctgagaagtttgcctttgg

SEQ ID NO: 74 - DNA, 20 bp, Mus musculus
aacttcctccttccccgtta

SEQ ID NO: 75 - DNA, 24 bp, Mus musculus
agggaatacactgtaagcctgtgt

SEQ ID NO: 76 - DNA, 24 bp, Mus musculus
ttaactactcggatcagacatcca

SEQ ID NO: 77 - DNA, 20 bp, Mus musculus
cccaggtgagcatagttggt

SEQ ID NO: 78 - DNA, 20 bp, Mus musculus
gggtgggtaaaatgggaact

SEQ ID NO: 79 - protein, 11 aa, Artificial Sequence
  The protein was designed and synthesized
ARTKQTARKST

SEQ ID NO: 80 - protein, 11 aa, Artificial Sequence
  The protein was designed and sythesized
ATGGVKKPHRY

SEQ ID NO: 81 - DNA, 22 bp, Mus musculus
ttcccagtccatccttttctcg

SEQ ID NO: 82 - RNA, 23 nt, Mus musculus
cuucaggaaccuugaguacugug

SEQ ID NO: 83 - RNA, 23 nt, Homo sapiens
cuucaggaaccucgaguacugug

SEQ ID NO: 84 - RNA, 23 nt, Homo sapiens
uucugaauuugcaaaguacugua

SEQ ID NO: 85 - DNA, 22 bp, Mus musculus
gggtatgtctcaaaggtacagc

SEQ ID NO: 86 - DNA, 21 bp, Mus musculus
ctgcgtgtatgtctgtgtgct